SEQUENCE LISTING (2 sequences)

SEQ ID NO: 1 - DNA, 36 bp, Artificial Sequence
  Synthetic construct
tgcgtagtctggagctatggguuuuagagcuagaaa

SEQ ID NO: 2 - protein, 8 aa, Artificial Sequence
  Synthetic construct
SIINFEKL

(19) United States
(12) Patent Application Publication
(10) Pub. No.: US 2023/0107770 A1
(43) Pub. Date: Apr. 6, 2023

(54) METHOD OF ENHANCING IMMUNOTHERAPY USING ER STRESS PATHWAY INHIBITORS

(71) Applicant: H. LEE MOFFITT CANCER CENTER AND RESEARCH INSTITUTE, INC., Tampa, FL (US)

(72) Inventors: Paulo C. RODRIGUEZ, Tampa, FL (US); Jose R. Conejo GARCIA, Tampa, FL (US); Frederick L. LOCKE, Lutz, FL (US)

(21) Appl. No.: 17/801,123

(22) PCT Filed: Feb. 22, 2021
(86) PCT No.: PCT/US2021/019064

§ 371 Date: Aug. 19, 2022

Related U.S. Application Data
(60) Provisional application No. 62/979,210, filed on Feb. 20, 2020.
(60) Provisional application No. 62/980,783, filed on Feb. 24, 2020.
(60) Provisional application No. 63/006,024, filed on Apr. 6, 2020.

(51) Int. Cl. C07K 14/47; A61K 35/17; A61K 45/06; A61P 35/00; C12N 9/12

(52) U.S. Cl. CPC C07K 14/4702; A61K 35/17; A61K 45/06; A61P 35/00; C07K 14/47; C12N 9/12; C12N 9/1205; C12Y 207/11; A61K 38/00

(57) ABSTRACT

Disclosed are compositions and methods for mediating immunosuppressive myelopoiesis. Additionally, disclosed herein are combination therapies for treating cancers and methods of using the same.

This application claims the benefit of U.S. Provisional Application No. 63/006,024, filed on Apr. 6, 2020, U.S. Provisional Application No. 62/980,783, filed on Feb. 24, 2020, and U.S. Provisional Application No. 62/979,210, filed on Feb. 20, 2020, applications which are incorporated herein by reference in their entireties.

This invention was made with government support under Grant No. R01CA184185, R01CA233512, R01CA103320, R01CA211229, R01CA157664, R01CA124515, and R01CA178687 awarded by the National Institutes of Health (NIH). The government has certain rights in the invention.

### I. BACKGROUND

The primary mechanisms supporting the immunoregulatory polarization of myeloid cells upon infiltration into tumors remain largely unexplored. Elucidation of these signals could enable better strategies to restore protective anti-tumor immunity. The tumor microenvironment (TME) disrupts the protein-folding capacity of the endoplasmic reticulum (ER) in infiltrating immune cells, thereby provoking a state of pathological “ER stress” that promotes immune evasion by cancer cells. What is needed is the development of successful T cell-based therapies for solid tumors, like ovarian carcinoma, will require overcoming the detrimental ER stress-driven immunoregulatory signals, which remain largely unexplored.

### II. SUMMARY

Disclosed are methods and compositions related to PKR-like ER kinase (PERK) inhibitors, endoplasmic reticulum (ER) stress inhibitors, and inositol-requiring enzyme 1α (IRE1α) inhibitors and methods of their use in immunotherapy.

In one aspect, disclosed herein are methods of reprogramming an immunosuppressive myelopoiesis in a tumor in a subject comprising administering to the subject a PERK inhibitor, ER stress inhibitor or IRE1α inhibitor.

Also disclosed herein method of reprogramming myeloid-derived suppressor cells (MDSC) in a tumor in a subject into immunostimulatory myeloid cells comprising administering to the subject a PERK inhibitor, ER stress inhibitor or IRE1α inhibitor.

In one aspect disclosed herein are methods of increasing the efficacy of an adoptive immunotherapy (such as, for example, administration of chimeric antigen receptor (CAR) T cells, CAR NK cells, tumor infiltrating lymphocytes (TILs), and/or marrow infiltrating lymphocytes (MILs)) said method comprising administering to the subject a PERK inhibitor, ER stress inhibitor or IRE1α inhibitor; wherein the administration of the PERK inhibitor, ER stress inhibitor or IRE1α inhibitor reprograms immunosuppressive myelopoiesis in a tumor thereby boosting the efficacy of the adoptive immunotherapy.

Also disclosed herein are methods of increasing the efficacy of an adoptive immunotherapy said method comprising a) obtaining a donor population of cells for immunotherapy (such as, for example, administration of chimeric antigen receptor (CAR) T cells, CAR NK cells, tumor infiltrating lymphocytes (TILs), and/or marrow infiltrating lymphocytes (MILs) from an autologous or allogeneic donor source); and b) contacting said cells with a increasing the efficacy of an adoptive immunotherapy; wherein the administration of the PERK inhibitor, ER stress inhibitor or IRE1α inhibitor reprograms the susceptibility of the donor cells to immunosuppressive myelopoiesis thereby boosting the efficacy of the adoptive immunotherapy. In one aspect, the donor population of cells are contacted with the increasing the efficacy of an adoptive immunotherapy ex vivo.

Also disclosed herein are methods of stimulating endogenous T cells (such as, for example TILs or MILs) in a subject to kill a tumor comprising administering to a subject a PERK inhibitor, ER stress inhibitor or IRE1α inhibitor wherein the administration of the PERK inhibitor, ER stress inhibitor or IRE1α inhibitor reduces or reduces the effects of one or more immunosuppressive elements in the tumor.

In one aspect, disclosed herein are methods of reprogramming an immunosuppressive myelopoiesis in a tumor of any preceding aspect, methods of reprogramming myeloid-derived suppressor cells (MDSC) in a tumor of any preceding aspect, methods of increasing the efficacy of an adoptive immunotherapy of any preceding aspect, and/or methods of stimulating endogenous T cells in a subject to kill a tumor of any preceding aspect, wherein the PERK inhibitor, ER stress inhibitor or IRE 1α inhibitor comprises a RNAi (for example and RNA that targets Nfe2l2 or Eif2ak3), small molecule (such as, for example the PERK inhibitor GSK-2606414 or AMG-44, Tauroursodeoxycholic acid (TUDCA), or the IRE1α inhibitor MKC8866, or KIRA8 or any ER stress inhibitor disclosed herein) peptide, protein, or antibody (such as, for example, an antibody that targets PERK, Gr1, IRE1α, Eif2ak3, CC12, or Nfe2l2).

Also disclosed herein are combination immunotherapies comprising an adoptive immunotherapy (such as, for example, administration of chimeric antigen receptor (CAR) T cells, CAR NK cells, tumor infiltrating lymphocytes (TILs), and/or marrow infiltrating lymphocytes (MILs)) and a PERK inhibitor, ER stress inhibitor, or IRE 1α inhibitor (including, but not limited to RNAi (for example and RNA that targets Nfe2l2 or Eif2ak3); small molecules (such as, for example the PERK inhibitor GSK-2606414 or AMG-44, Tauroursodeoxycholic acid (TUDCA), or the IRE1α inhibitor MKC8866, or KIRA8 or any ER stress inhibitor disclosed herein) peptides; proteins; or antibodies that target PERK, Gr1, Eif2ak3, CC12, or Nfe2l2; or tumor-infiltrating Eif2ak3^{KO-Lyz2} CD11b⁺ Gr1⁺ cells).

In one aspect, disclosed herein are methods of treating, inhibiting, reducing, ameliorating, and/or preventing a cancer and/or metastasis in a subject comprising administering to a subject the combination therapy of any preceding aspect. For example, disclosed herein are methods of treating, inhibiting, reducing, ameliorating, and/or preventing a cancer and or metastasis in a subject comprising administering to a subject an (such as, for example, administration of chimeric antigen receptor (CAR) T cells, CAR NK cells, tumor infiltrating lymphocytes (TILs), and/or marrow infiltrating lymphocytes (MILs)) and a PERK inhibitor, ER stress inhibitor, or IRE1α inhibitor (including, but not limited to RNAi (for example and RNA that targets Nfe2l2 or Eif2ak3); small molecules (such as, for example the PERK inhibitor GSK-2606414 or AMG-44, Tauroursodeoxycholic acid (TUDCA), or the IRE1α inhibitor MKC8866, or KIRA8 or any ER stress inhibitor disclosed herein) peptides; proteins; or antibodies that target PERK, Gr1, IRE1α, Eif2ak3, CC12, or Nfe2l2; or tumor-infiltrating Eif2ak3^{KO-Lyz2} CD11b⁺ Gr1⁺ cells). In one aspect, the CAR T cells, CAR NK cells, TILs, and MILs can be obtained from a donor source including, but not limited to autologous or allogeneic donors. I

Also disclosed herein are methods of treating, inhibiting, reducing, ameliorating, and/or preventing a cancer and or metastasis of any preceding aspect, wherein the CAR T cells, CAR NK cells, TILs, and MILs that comprise the adoptive immunotherapy are contacted with the PERK inhibitor, ER Stress inhibitor, and/or an IRE1α inhibitor ex vivo prior to administration to the subject.

Also disclosed herein are methods of treating, inhibiting, reducing, ameliorating, and/or preventing a cancer and or metastasis of any preceding aspect, wherein the CAR T cells, CAR NK cells, TILs, and MILs that comprise the adoptive immunotherapy are contacted with the PERK inhibitor, ER Stress inhibitor, and/or an IRE1α inhibitor in vivo.

Also disclosed herein are engineered immune cells (such, as, for example, CAR NK cell, CAR NK T cell, or CAR T cells) transduced to express Chimeric Endocrine Receptors (CERs) that express one or more subunits of the follicle-stimulating hormone (FSH).

In one aspect, disclosed herein are methods of treating, inhibiting, reducing, ameliorating, and/or preventing a cancer and/or metastasis comprising a FSH-receptor positive (FSHR+) tumors in a subject said method comprising administering to the subject a the engineered immune cell of any preceding aspect.

Also disclosed herein are methods of increasing the efficacy of an engineered immune cell of any preceding aspect said method comprising contacting the immune cell with a PERK inhibitor, ER stress inhibitor, and/or IRE1α inhibitor.

In one aspect, disclosed herein are methods of assessing responsiveness to adoptive immunotherapy comprising obtaining adoptively transferred immune cells from a recipient subject and measuring the amount of spliced XBP-1 in the adoptively transferred immune cells, wherein a high level of XBP-1 relative to a control indicates the subject is not responsive to the adoptive immunotherapy.

Also disclosed herein are methods of treating, inhibiting, reducing, ameliorating, and/or preventing a cancer and or metastasis in a subject comprising administering to a subject an adoptive immunotherapy and monitoring the amount of spliced XBP-1 in the adoptively transferred immune cells, wherein the level of spliced XBP-1 relative to a control is indicative of responsiveness to the adoptive immunotherapy; and wherein a high level of spliced XBP-1 relative to a control indicates that the recipient subject is not responsive to the adoptive immunotherapy.

In one aspect, disclosed herein are methods of treating, inhibiting, reducing, ameliorating, and/or preventing a cancer and or metastasis of any preceding aspect, wherein the level of spliced XBP-1 relative to a control is high, said method further comprises administering to the subject a PERK inhibitor, ER Stress inhibitor, and/or an IRE1α inhibitor.

Also disclosed herein are methods of treating, inhibiting, reducing, ameliorating, and/or preventing a cancer and or metastasis comprising administering to the subject a PERK inhibitor, ER Stress inhibitor, and/or an IRE1α inhibitor.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A, 1B, 1C, 1D, 1E, 1F, 1G, and 1H show that TME drives immunosuppressive function and UPR activation in tumor-MDSC. FIG. 1A shows a representative result from 3 independent repeats showing the regulatory effect of splenic-MDSC or tumor-MDSC from mice bearing LLC (left), B16 (middle) or ID8-Defb29/Vegf-a (right) tumors; and from splenic CD11b⁺ Gr1⁺ cells (iMC) from tumor-free mice on the proliferation of CFSE-labeled T cells primed with plate-bound anti-CD3/CD28. T cell: MDSC co-cultures were established at 1:1/4 ratio and T cell proliferation assessed 72 hours post-priming by flow cytometry. FIG. 1B shows immunoblots for phospho-PERK (pPERK), PERK, phospho-IRE1α (pIRE1α), and IRE1α in tumor-MDSC, splenic-MDSC, and iMC from A. FIG. 1C shows representative ER-Tracker fluorescence histograms and mean fluorescence intensity (MFI) of iMC, splenic-MDSC, and tumor-MDSC from LLC bearing mice detected by flow cytometry and compared to fluorescence minus one (FMO) (n=3) (left). Merging of the obtained data (right). FIG. 1D show illustrative transmission electron microscopy (TEM) from iMC, splenic-MDSC, and tumor-MDSC. Red arrow heads point to the morphology of the ER (n=3) (left). Scale bar 2 μm (upper) and 200 nm (lower). Quantification of the data (right) (1E and 1F) BM-MDSC developed after culture of BM precursors for 96 hours with G-CSF and GM-CSF (20 ng/ml each) and in the presence of 30% LLC-TES or Thaps (200 nM, last 24 hours) were tested for pPERK, PERK, pIRE1α and IRE1α expression (1E); and immunosuppressive activity (1:1/8) (1F). Results are a representative finding from 3 repeats. FIGS. 1G and 1H show human-MDSC derived from BM-precursors cultured with GM-CSF plus IL-6 (10 ng/ml each) for 6 days and in the presence of 10-60% supernatants from the renal cell carcinoma cell line 786-0 or with Thaps (last 24 hours) were tested for expression of pPERK, PERK, pIRE1α, and IRE1α (1G); or co-cultured with allogeneic primed CFSE-labeled T cells (anti-CD3/CD28, ratio 2:1) (1H). T cell proliferation was evaluated 72 hours later by flow cytometry. Representative histograms are from 3 independent repeats. Statistics were applied using one way ANOVA, *, p<0.05; **, p<0.01.

FIGS. 2A, 2B, 2C, 2D, 2E, 2F, 2G, 2H, 2I, 2J, 2K, and 2L show that TUDCA impairs MDSC-related T cell dysfunction and boosts immunotherapy. FIG. 2A shows tumor growth kinetics±s.e.m in mice bearing s.c. LLC (left) or B16 (right) tumors and treated daily after day 6 post-tumor injection with vehicle (saline) or TUDCA (250 mg/kg). n=10. FIG. 2B shows CFSE-labelled T cells primed with plate-bound anti-CD3/CD28 were co-cultured with tumor-MDSC (1:1/4) from LLC bearing mice treated with vehicle or TUDCA. MDSC were sorted after 15 days of tumor injection. Bars are mean of T cell proliferation±s.e.m. of n=5. FIG. 2C shows Representative expression of NOS2 and Arginase I in tumor-MDSC from A. FIG. 2D shows the percentage of CD69⁺CD44⁺ in gated tumor-CD8⁺ T cells (TILs) from LLC and B16 bearing mice treated with vehicle or TUDCA as in (2A). n=5. FIG. 2E shows LLC tumor growth in wildtype (WT) and Rag1^{KO} mice treated with vehicle or TUDCA as in (2A). FIG. 2F shows tumor growth kinetics±s.e.m in mice bearing LLC (left) or B16 (right) tumors treated as in (2A) and additionally receiving or not 250 μg anti-Gr1 starting the same day of tumor injection and followed every 3^{rd} day until tumor endpoint. n=5/group. FIG. 2G shows the percentage of CD69⁺CD44⁺ in CD8⁺ TILs from B16 bearing mice treated as in (2F). FIG. 2H shows the percentages of EGSRNQDWL-H-2D^{b} tetramer⁺ cells in CD8 TILs from B16 bearing mice treated as in 2F. FIG. 2I shows tumor growth kinetics±s.e.m in mice bearing B16 tumors treated as in (2A) and additionally receiving or not 250 μg anti-PD-L1 starting on day 6 post-tumor injection and followed every 3^{rd} day until tumor endpoint. n=10/group. FIG. 2J shows mice bearing s.c. EG7 tumors for 6 days, received daily doses of TUDCA or vehicle as in (2A). Additionally, specific cohorts received 1×10⁶ CD8⁺ OT-I T cells pre-activated with OVA257-264 for 48 hours. Results are mean of tumor volume±s.e.m in 5 mice/group. FIG. 2K shows Spleens of mice from (2H) were harvested 5 days after T cell transfer and tested for IFNγ production by EliSpot upon activation with OVA257-264 for 24 hours (n=3 repeats of 3 spleens/group). FIG. 2L shows tumor volume±s.e.m for EG7 or LLC tumors injected s.c. in opposite flanks of mice that previously rejected EG7 tumors after treatment TUDCA+ACT (n=3). Statistics were applied using one way ANOVA or student's t-test, *, p<0.05; **, p<0.01.

FIGS. 3A, 3B, 3C, 3D, 3E, 3F, 3G, 3H, 3I, 3J, 3K, 3L, 3M, 3N, and 3O show that activation of PERK intrinsically controls MDSC function. FIG. 3A shows from left to right: Representative images (20× resolution and further 10× digital magnification) showing phospho-PERK (pPERK, Magenta), CD11b (Red), CD15 (Yellow), CD14 (Green), HLA-DR (Orange), pan-Cytokeratin (pCK, Cyan), and DAPI (Blue) in a TMA containing healthy lung (n=8) and metastatic non-small lung cancer (Met-NSCLC) tissues (n=46) by Vectra Automated Multispectral Imaging. Representative histogram showing the expression of pPERK on pCK^{neg}CD11b⁺HLA-DR^{neg} cells from healthy lung (grey) or MET-NSCLC (red). Merged frequencies of phospho-PERK⁺ cells in gated pCK^{neg} CD11b⁺HLA-DR^{neg} CD14^{neg} CD15⁺ (PMN-MDSC-LC) and pCK^{neg} CD11b⁺HLA-DR^{neg} CD14⁺CD15^{neg} (M-MDSC-LC) from TMA cores from healthy lung or MET-NSCLC. Scatter plots show the mean±s.e.m, **, p<0.01, *, p<0.05 by unpaired Student's t-test. FIG. 3B shows volume±s.e.m for LLC (left, n=20) and B16 (right, n=10) tumors injected into Eif2ak3^{Flox} and Eif2ak3^{KO-Lyz2} mice.

FIG. 3C shows tumor kinetics of autochthonous flank sarcoma developed after injection with adenovirus expressing Cre recombinase in the right flanks of K-Ras^{G12D+} Trp53^{flox/flox} mice previously reconstituted with bone marrows from Eif2ak3^{Flox} or Eif2ak3^{KO-Lyz2} mice (n=4). FIG. 3D shows representative data (n=3) of anti-CD3/CD28-activated CFSE-labeled T cells co-cultured with tumor-MDSC (ratio 1:1/4) from LLC-bearing Eif2ak3^{Flox} or Eif2ak3^{KO-Lyz2} mice. MDSC were isolated 15 days post-tumor injection and T cell proliferation assessed 72 hours after co-culture by flow cytometry. FIG. 3E shows NOS2 and Arginase I (left) and pPERK, PERK, phospho-IRE1α (pIRE1α), and IRE1c (right) in tumor-MDSC from Eif2ak3^{Flox} and Eif2ak3^{KO-Lyz2} mice bearing LLC tumors for 15 days. FIG. 3F shows BM-MDSC from Eif2ak3^{Flox} or Eif2ak3^{KO-Tek} mice pre-treated or not with Thaps for 24 hours were co-cultured with CFSE-labelled T cells primed with anti-CD3 and CD28 (0.5-0.06:1). T cell proliferation was tested 72 hours later by flow cytometry. FIGS. 3G, 3H, and 3I show the percentage of CD8⁺ T cell infiltration (3G), and CD69⁺CD44⁺ (3H) and IFNγ⁺ cells (3I) in CD8⁺ TILs from LLC tumors injected into Eif2ak3^{Flox} and Eif2ak3^{KO-Lyz2} mice was tested by flow cytometry. Bars are mean±s.e.m on n=5. FIG. 3J shows LLC tumor growth in Eif2ak3^{Flox} or Eif2ak3^{KO-Lyz2} mice treated with 400 μg anti-CD8 antibody or isotype every 3^{rd} day. Tumor volume±s.e.m in 5 mice/group. FIG. 3K shows tumor growth kinetics±s.e.m in B16-bearing mice treated with vehicle or AMG-44 (12, 24 mg/kg) starting 6 days post-tumor injection and continued daily until endpoint (n=5). FIG. 3L shows the percentage of proliferating CFSE-labeled T cells co-cultured with tumor-MDSC (1:1/4) sorted from B16-bearing mice treated with vehicle or AMG-44 (12 mg/kg). Bars are mean±s.e.m of 3 repeats. FIG. 3M shows the frequency of IFNγ⁺ in CD8⁺ TILs from mice treated with vehicle or AMG-44 (12 mg/kg). FIG. 3N shows the tumor growth kinetics±s.e.m in mice bearing B16 tumors treated with vehicle or AMG-44 (12 mg/kg) and additionally receiving or not 250 μg anti-PD-L1 starting on day 6 post-tumor injection and followed every 3^{rd} day until tumor endpoint. n=5/group. FIG. 3O shows the percentage of proliferation of anti-CD3/CD28 primed CFSE-labeled T cells co-cultured with control or Thaps-treated BM-MDSC (1:1/8) previously exposed for 3 hours to 5 μM AMG-44. For tumor studies, data are presented as average tumor volume±s.e.m. Statistics were one way ANOVA student's t-test or log-rank (Mantel-Cox)*, p<0.05; **, p<0.01.

FIGS. 4A, 4B, 4C, 4D, 4E, 4F, 4G, 4H, 4I, 4J, 4K, and 4L show that PERK deletion functionally reprograms tumor-MDSC. (A-B) LLC growth kinetics±s.e.m in Eif2ak3^{Flox} and Eif2ak3^{KO-Lyz2} mice treated or not with 250 μg anti-Gr1 (4A), 250 μg anti-CCL2 (4B) or their day corresponding isotypes starting the same day of tumor injection and followed every 3^{rd} until tumor endpoint. n=5 per group. FIG. 4C shows tumor growth in C57BL/6 (left) and immunodeficient Rag1^{KO} (right) mice injected with LLC cells alone or co-injected at a 1:1 ratio with tumor-MDSC collected from Eif2ak3^{flox} or Eif2ak3^{KO-Lyz2} mice bearing LLC tumors. Mean±s.e.m from 5 mice/group. FIG. 4D shows a heat-map representing the relative expression of selected markers on tumor M-MDSC and PMN-MDSC from Eif2ak3^{KO-Lyz2} mice compared to Eif2ak3^{Flox} controls (n=10). Results were obtained by flow cytometry and calculated by dividing the mean fluorescence intensity (MFI) by the background of fluorescence minus one (FMO). FIG. 4E shows the percentage of IL-12 (left) and TNF-α (right) in M-MDSC and PMN-MDSC from LLC tumors previously injected into Eif2ak3^{Flox} or Eif2ak3^{KO-Lyz2} mice. Bars are mean±s.e.m of n=3. FIG. 4F shows representative histograms of proliferating CFSE-labeled OT-I CD8⁺ T cells co-cultured with LLC-infiltrating MDSC from Eif2ak3^{Flox} or Eif2ak3^{KO-Lyz2} mice, previously loaded with complete OVA for 16 hours. Positive controls were GM-CSF plus IL-4-developed DCs loaded for 16 hours with 1 μg/ml OVA. FIG. 4G shows merged data from MFI±s.e.m (left) and representative histogram of ZsGreen fluorescence (right) in tumor-MDSC from Eif2ak3^{Flox} and Eif2ak3^{KO-Lyz2} mice. n=5. FIG. 4H shows representative result of OVA-bound H-2K^{b} in tumor-MDSC from Eif2ak3^{Flox} and Eif2ak3^{KO-Lyz2} mice (right). MFI±s.e.m from n=8 (left). FIG. 4I shows the percentage of SIINFEKL-H-2K^{b}-tetramer⁺ cells in CD8⁺ TILs from Pan02-Ova-ZsGreen tumors injected into Eif2ak3^{Flox} or Eif2ak3^{KO-Lyz2} mice. FIGS. 4J and 4K show fold increase of ZsGreen fluorescence (4J) and SIINFEKL-bound H-2k^{b} (4K) MFI in tumor-associated M-MDSC, PMN-MDSC, macrophages, and myeloid DCs from Eif2ak3^{KO-Lyz2} mice relative to Eif2ak3^{Flox} controls. Results were obtained by flow cytometry and calculated by dividing the mean fluorescence intensity (MFI) of the specific myeloid populations from Eif2ak3^{KO-Lyz2} mice over those from Eif2ak3^{Flox} controls. FIG. 4L shows representative proliferation of cell trace violet-labeled naïve OT-I CD8⁺ T cells co-cultured with tumor-MDSC (1:1/4) from Pan02-Ova-ZsGreen-bearing Eif2ak3^{Flox} or Eif2ak3^{KO-Lyz2} mice (n=3). As a positive control for OT-1 proliferation, naïve OT-1 cells were co-cultured with SIINFEKL-loaded DCs developed from GM-CSF plus IL-4 treated bone marrows of tumor-free mice (1:1/4). Statistics were applied using one way ANOVA or student's t-test, *, p<0.05; **, p<0.01.

FIGS. 5A, 5B, 5C, 5D, 5F, 5G, 5H, 5I, 5J, and 5K show functional switch of PERK-null MDSC occurs through impaired NRF2 signaling. FIG. 5A shows immunoblot of CHOP in tumor-MDSC from Eif2ak3^{Flox} and Eif2ak3^{KO-Lyz2} mice bearing LLC tumors for 15 days. Result is a representative finding of n=5. FIG. 5B shows CFSE-labelled T cells primed with plate-bound anti-CD3/CD28 were co-cultured with wild type (WT), Eif2ak3^{KO-Tek} or Ddit3^{KO} BM-MDSC previously treated or not with Thaps for 24 hours (ratio 1:1/8). Bars are mean T cell proliferation±s.e.m. (n=3) after 72 hours of co-culture. FIG. 5C shows BM-MDSC from Eif2ak3^{Flox} or Eif2ak3^{KO-Lyz2} mice were transduced for 72 hours with lentivirus expressing Ddit3 (Ddit3-OE) or control (control-OE), after which they were treated with or without Thaps for 24 hours, and then co-cultured with CFSE-labeled T cells primed with anti-CD3/CD28 (1/8:1). T cell proliferation was monitored 72 hours later by flow cytometry. Bars are mean of T cell proliferation±s.e.m (n=3). FIGS. 5D and 5E show NRF2 expression by western blot (Illustrative from n=3) (5D) and NRF2 binding to a consensus DNA-binding sequence using 15 μg of protein (5E) in nuclear extracts from tumor-MDSC from LLC tumors growing for 15 days in Eif2ak3^{Flox} or Eif2ak3^{KO-Lyz2} mice. FIG. 5F shows representative DCFDA histograms in control MDSC (left), BM-MDSC treated with Thaps (middle), or tumor-MDSC (right) from Eif2ak3^{Flox} or PERK-deficient mice. FIG. 5G shows BM-MDSC from Eif2ak3^{Flox} or Eif2ak3^{KO-Tek} mice were pre-treated for 3 hours with vehicle, Resveratrol, or Sulforaphane (SFRN) before incubating with Thaps for 24 hours. Then, ability of MDSC to block proliferation of primed CFSE-labeled T cells was tested (1/8:1). FIG. 5H shows BM-MDSC were transduced for lentivirus coding for NRF2-ΔNeh2 or control, followed by culture in the presence or the absence of Thaps for 24 hours. Then, BM-MDSC were co-cultured with activated CFSE-labeled T cells and proliferation evaluated 72 hours later. FIG. 5I shows LLC growth kinetics±s.e.m in Eif2ak3^{Flox} or Eif2ak3^{KO-Lyz2} mice treated with either vehicle or SFRN (n=5). FIG. 5J shows the percentages of DCFDA in MDSC from (5I). FIG. 5K shows the percentages of proliferating T cells co-cultured with MDSC (1:1/4) from LLC tumors from (5I). Statistics were applied using one way ANOVA or student's t-test, *, p<0.05; **, p<0.01, ***, p<0.001.

FIGS. 6A, 6B, 6C, 6D, 6E, 6F, 6G, 6H, and 6I shows that NRF2 regulates mitochondrial homeostasis in PERK-null MDSC. FIG. 6A shows representative transmission electron microscopy (TEM) image of tumor-MDSC from LLC tumors developed in Eif2ak3^{Flox} or Eif2ak3^{KO-Lyz2} mice (n=3). Arrow heads point to the morphology of mitochondria. Scale bar 500 nm. FIG. 6B shows illustrative histogram (upper) and merged MFI values (lower) of Mitotracker fluorescence in tumor-MDSC from Eif2ak3^{Flox} and Eif2ak3^{KO-Lyz2} mice. FIG. 6C shows representative histogram of aggregated JC-1 fluorescence (upper) and JC-1 aggregates: JC-1 monomers (lower) in tumor-MDSC from Eif2ak3^{Flox} and Eif2ak3^{KO-Lyz2} mice. FIG. 6D shows Oxygen consumption rate (OCR) after mitochondrial stress analysis in tumor-MDSC from LLC-bearing Eif2ak3^{Flox} and Eif2ak3^{KO-Lyz2} mice (n=5). FIG. 6E shows OCR after mitochondrial stress analysis in BM-MDSC transduced for 72 hours with lentivirus carrying NRF2-ΔNeh2 or control sequences and treated for the last 24 hours of culture with Thaps. Representative of 3 repeats±s.e.m. FIG. 6F shows OCR as in (6D) in tumor-MDSC from Eif2ak3^{Flox} and Eif2ak3^{KO-Lyz2} mice treated with vehicle or SFRN. n=3/group. FIGS. 6G and 6H show quantitative PCR for mitochondrial Coxa and Nd1 genes in mitochondria-free cytosolic fractions (6G) and mitochondrial-enriched fractions (6H) from tumor-MDSC of LLC-bearing Eif2ak3^{Flox} or Eif2ak3^{KO-Lyz2} mice. Results are triplicates of 5 samples per group. FIG. 6I shows BM-MDSC from Eif2ak3^{Flox} or Eif2 ak3^{KO-Tek} mice were developed in the presence of 150 ng/ml of ethidium bromide (EtBr), followed by culture in the presence or the absence of Thaps for 24 hours. Then, BM-MDSC were co-cultured with activated CFSE-labeled T cells and proliferation evaluated 72 hours later. Results are representative of 3 independent experiments. Statistics were applied using student's t-test, *, p<0.05; **, p<0.01.

FIGS. 7A, 7B, 7C, 7D, 7E, 7F, 7G, 7H, 7I, 7J, and 7K show that STING-dependent Type I IFNs regulates functional switch of PERK-null MDSC. FIG. 7A shows the expression of cytosolic phospho-TBK1 (pTBK1) and total TBK1, or nuclear phospho-IRF3 (pIRF3) and IRF3 in MDSC infiltrating LLC tumors from Eif2ak3^{Flox} and Eif2ak3^{KO-Lyz2} mice. Image is a representative result from n=3. FIG. 7B shows LLC tumor growth kinetics±s.e.m. in Lyz2^{Cre}, Eif2ak3^{KO-Lyz2}, Tmem173^{KO-Lyz2} and Eif2ak3-Tmem173^{KO-Lyz2} mice (n=7). FIG. 7C shows the percentages of proliferating anti-CD3/CD28 primed CFSE-labeled T cells cultured with tumor-MDSC from (7B) (1:1/4). FIG. 7D shows the percentage of IFNγ⁺ in CD8⁺ TILs from (7B). FIG. 7E shows Pan02-Ova-ZsGreen tumors were injected into Lyz2^{Cre}, Eif2ak3^{KO-Lyz2}, Tmem173^{KO-Lyz2} and Eif2ak3-Tmem173^{KO-Lyz2} mice, and 14 days later, tumor-draining lymph nodes were tested for IFNγ by EliSpot upon activation with OVA257-264 for 24 hours (n=3 mice). FIG. 7F shows representative proliferation of cell trace violet-labeled naïve OT-I CD8⁺ T cells co-cultured with tumor-MDSC from Pan02-Ova-ZsGreen-bearing mice from (7E). FIG. 7G shows relative expression of Ifnβ1, Isg15, Ifit3, Cxcl10 mRNAs in MDSC from tumors from (7B). Results are mean of duplicates from 5 samples per group. FIG. 7H shows tumor growth±s.e.m. in LLC-bearing Eif2ak3^{Flox} and Eif2ak3^{KO-Lyz2} mice treated or not with 1 mg anti-IFN alpha/beta receptor subunit 1 (IFNAR1) blocking antibody every 3 days and starting on day 0 of tumor injection (n=5). FIG. 7I shows BM-MDSC were developed in the presence of 150 ng/ml of ethidium bromide (EtBr), followed by culture in the presence or the absence of Thaps for 24 hours. Then, Ifnβ1 mRNA levels were evaluated. Results are representative of 3 independent experiments. FIG. 7J shows quantitative qPCR of Ifnβ1 mRNA in BM-MDSC transduced for 72 hours with lentivirus coding for NRF2-ΔNeh2 or control sequences and then treated with Thaps for the last 24 hours. FIG. 7K shows relative expression of Ifnβ1 mRNA in LLC tumor-MDSC from Eif2ak3^{Flox} and Eif2ak3^{KO-Lyz2} mice treated with vehicle or SFRN. Results are presented as tumor volume and plotted as mean±s.e.m. Statistics were applied using one way ANOVA, *, p<0.05; **, p<0.01.

FIGS. 8A, 8B, 8C, 8D, 8E, and 8F show that activation of the UPR drives immunosuppressive potential of tumor-MDSC. FIG. 8A shows the frequency of PMN-MDSC (Ly6G⁺Ly6C^{low}) and M-MDSC (Ly6G^{neg}Ly6C^{high}) were detected in gated CD11b+Gr1+ cells from spleens and tumors of LLC-bearing mice or spleens from mice without tumors (n=5). FIG. 8A shows the percentages of ER-tracker high cells from iMC, splenic-MDSC and tumor-MDSC. ER-tracker high cutoff was defined as the average plus 2 standard deviations for the ER tracker MFI values of iMC (MFI≥2680 in the LLC model). FIG. 8C shows illustrative histograms of the ER-tracker fluorescence of iMC, splenic-MDSC and tumor-MDSC from B16 tumors (left) and ascites-associated MDSC from ID8-Defb29/Vegf-a (right) detected by flow cytometry and compared to fluorescence minus one (FMO) (n=3). FIG. 8D shows LLC tumor growth in vehicle and Thaps treated mice. Results are presented as tumor volume mean±s.e.m. (n=5). FIG. 8E shows representative flow cytometry and quantification of the proportions of MDSC, PMN-MDSC and M-MDSC in spleens of tumor-free and LLC-bearing mice treated with vehicle or Thaps. FIG. 8F shows representative histograms of CFSE dilution of T cells co-cultured with tumor-MDSC from LLC-bearing mice treated with vehicle or Thaps. Results are representative of 3 repeats per group. Statistics were applied using one way ANOVA, *, p<0.05; **, p<0.01.

FIGS. 9A, 9B, 9C, 9D and 9E show that TUDCA blocks MDSC activity. FIG. 9A shows CFSE-labelled T cells primed with plate-bound anti-CD3/CD28 were co-cultured with tumor-MDSC (1:1/4) from B16 bearing mice treated with vehicle or TUDCA. MDSC were sorted after 15 days of tumor injection. Bars are mean of T cell proliferation±s.e.m. of n=5. FIG. 9B shows immunoblots for phospho-PERK (pPERK), PERK, phospho-IRE1α (pIRE1α), and IRE1α in tumor-MDSC from mice bearing LLC (left) and B16 (right) from A. FIG. 9C shows representative histograms of proliferation of human T cells co-cultured with human GM-CSF plus IL-6 BM-derived MDSC incubated with or without TES and in the absence or the presence of TUDCA. Results are representative of 3 independent repeats. FIG. 9D shows representative histograms of proliferating mouse T cells co-cultured with BM precursors incubated with or without TES and in the absence or the presence of TUDCA. Results are representative of 3 independent repeats. FIG. 9E shows representative histograms of proliferating mouse T cells co-cultured with tumor-MDSC pre-treated ex-vivo with or without TUDCA for 24 hours (n=3).

FIGS. 10A, 10B, 10C, 10D, 10E, 10F, 10G, 10H, 10I, 10J, 10K, 10L, 10M, 10N, 10O, 10P, 10Q, 10R, and 10S show that PERK deletion and inhibition reprograms MDSC regulatory activity. FIG. 10A shows merged frequencies of phospho-PERK+ cells in gated pCK^{neg} CD14+CD15neg HLA-DR^{neg} (M-MDSC-LC, left) and pCKneg CD14neg CD15+ HLA-DR^{neg} (PMN-MDSC-LC, right) from TMA cores from healthy ovary (n=8) or Ovarian Carcinoma tumors (n=84). Scatter plots show the mean±s.e.m. *p<0.05 by unpaired Student's t-test. FIG. 10B shows representative gating strategy and quantification of the percentages Td-Tomato+ cells among different tumor-associated myeloid populations from Lyz2-Td-Tomato reporter mice bearing LLC tumors for 15 days. FIG. 10C shows Eif2qak3 mRNA in myeloid populations infiltrating LLC tumors. (n=5). FIG. 10D shows survival of Eif2ak3^{Flox} and Eif2ak3^{KO-Lyz2} mice bearing i.p. ID8-Defb29/Vegf ovarian tumors (n=4). FIG. 10E shows the percentage of MDSC (left), M-MDSC (middle) and PMN-MDSC (right) in LLC tumors injected for 15 days into Eif2ak3^{Flox} and Eif2ak3^{KO-Lyz2} mice. FIGS. 10F and 10G show the percentages of Annexin V+ MDSC (10F) and DR+M-MDSC and PMN-MDSC (10G) within LLC tumors of Eif2ak3^{Flox} and Eif2ak3^{KO-Lyz2} mice. FIGS. 10H and 10I show immunoblots of pPERK, PERK, pIRE1α and IRE1α (10H) and Eif2ak3 mRNA levels (10I) from BM cells (baseline) or BM-MDSC from Eif2ak3^{Flox}, Eif2ak3^{KO-Lyz2} and Eif2ak3m-Tek mice treated or not with Thaps for 24 hours. n=3 mice. FIG. 10J shows the percentages of Td-Tomato+ cells in different myeloid cells from the bone marrow of Lyz2-T-tomato and Ted-d-Tomato reporter mice (10K) Eif2ak3 mRNA expression in BM-myeloid populations from Eif2ak3^{Flox}, Eif2ak3^{KO-Lys2} and Eif2ak3^{KO-Tek} mice (n=3). FIG. 10L shows Flox and PERK-deficient mice were s.c. injected with LLC tumors and 15 days later, BM were collected and tested for the frequency of GMP (Lin^{neg}c-kit⁺Sca-1^{neg}CD34⁺CD16/32^{hi}) or CMP (Li^{neg}c-kit⁺Sca-1^{neg}CD34⁺CD16/32^{low}) n=5 repeats. FIG. 10M shows hematopoietic precursors (Lin^{neg} (CD5, B220, CD11b, Gr-1, Ter-119, c-kit+ cells) were isolated from bone marrow of flox and PERK-null mice bearing LLC tumors and 2.5×10⁵ cells cultured for 96 hours in GM-CSF/G-CSF (20 ng/ml each). Then, numbers of MDSC (CD11b⁺Gr1⁺), M-MDSC (CD11b⁺Ly6G^{neg}Ly6C^{high}) and PMB-MDSC (CD11b⁺Ly6G⁺Ly6C^{low}) were calculated. N=5. FIG. 10N shows B16 tumor growth in mice treated with vehicle or GSK-2606414 (n=5). Results are presented as tumor volume and plotted as mean±s.e.m. FIG. 10O shows the percentages of T cell proliferation after co-cultures with MDSC from tumors of vehicle or GSK-2606414 treated mice. FIG. 10P shows the kinetics of blood glucose levels in B16-bearing mice treated with vehicle or AMG-44 (12 mg/kg or 24 mg/kg). FIGS. 10Q and 10R show representative area (10Q) and expression of Insulin (10R) (200 μm scale) and corresponding merged results in pancreatic islets from B16-bearing mice treated with vehicle or AMG-44. n=5. FIG. 10S shows tumor growth curves from Eif2ak3^{Flox} or Eif2ak3^{KO-Lyz2} mice bearing B16 tumors treated with vehicle or AMG-44 (12 mg/kg) (n=5). Statistics were applied using one way ANOVA or student's t-test, *, p<0.05; **, p<0.01.

FIGS. 11A, 11B, 11C, and 11D show that PERK deletion fails to prime cross-presentation of CD4+ T cells by tumor-MDSC. FIG. 11A shows Pan02-OVA-ZsGreen tumors were injected into Eif2ak3^{KO-Lys2} or Eif2ak3^{Flox} mice, and 18 days later, tumor suspensions were processed and CD11b+Gr+ cells tested for the presence of ZsGreen and SIINFEKL (SEQ ID NO: 2)-bound H-2K^{b}. N=4 repeats. FIG. 11B shows representative histograms illustrating the proliferation of cell trace violet-labeled naïve OT-II CD4+ T cells co-cultured with MDSC sorted from Pan02-Ova-ZsGreen tumors from Eif2ak3^{Flox} or Eif2ak3^{KO-Lyz2} mice or control DCs (n=3). FIG. 11C shows expression of SIINFEKL-bound H-2Kb in tumor-MDSC from Pan02-Ova-ZsGreen-bearing mice treated with vehicle or TUDCA (n=5). FIG. 11D shows representative proliferation of cell trace violet-labeled naïve OT-I CD8+ T cells co-cultured with tumor-MDSC (1:1/4) from Pan02-Ova-ZsGreen-bearing mice treated as in C (n=3).

FIGS. 12A, 12B, 12C, 12D, 12E, 12F, and 12G show that PERK deletion impairs MDSC activity through decreased NRF2 signaling. FIG. 12A shows Ddit3 mRNA expression in BM-MDSC transduced for 72 hours with lentivirus coding for Ddit3 (Ddit3-OE) or control (control-OE) sequences. Results are duplicates of 3 repeats. FIG. 12B shows immunoblot of NRF2 in nuclear extracts from splenic-MDSC, tumor-MDSC from LLC tumor bearing mice; and in iMC from naïve mice. FIG. 12C shows representative histograms of proliferating T cells co-cultured with tumor-MDSC sorted from control, Eif2ak3^{KO-Lyz2} and Nfe2l2^{KO} bearing LLC tumors (1:1/4) (n=3). FIGS. 12D and 12E shows NRF2 immunoblot (12D) and NRF2 binding to a consensus DNA sequence (12E) in nuclear extracts (15 μg) of BM-MDSC treated or not with Thaps. FIG. 12F shows Nef2l2 mRNA levels in BM-MDSC transduced for 72 hours with lentivirus coding for NRF2-ΔNeh2 or control. n=3. FIG. 12G shows ROS levels as presented by DHE in MDSC from FIG. 5H. Statistics were applied using one way ANOVA, **, p<0.01.

FIGS. 13A, 13B, 13C, 13D, and 13E show mitochondrial dysfunction in PERK-null MDSC is mediated by lower NRF2. FIG. 13A shows the oxygen consumption rate (OCR) after mitochondrial stress profile of BM-MDSC from Eif2ak3^{Flox} or Eif2ak3m-Tek mice previously cultured in the presence or absence of Thaps for 24 hours. Measurements were done in triplicates. Presented profile is a representative of 3 repeats. FIG. 13B shows ECAR by glycolysis stress profile of tumor-MDSC from LLC-bearing Eif2ak3^{Flox} or Eif2ak3^{KO-Lyz2} mice (n=3). FIG. 13C shows OCR from BM-MDSC transduced for 72 hours with lentivirus carrying NRF2-ΔNeh2 or control sequences. FIGS. 13D and 13E show quantitative PCR for Cox1 (13D) and Nd1 (13E) genes in BM-MDSC from Eif2ak3Flox or Eif2ak3KO-Tek mice at baseline or after treatment with Thaps for 3 hours. Statistics were applied using student's t-test, **, p<0.01.

FIGS. 14A, 14B, 14C, and 14D show the role of STING in the alterations induced after PERK-deletion in MDSC. FIG. 14A shows CD8+ T cells in LLC tumors developed for 15 days in controls, Eif2ak3-Tmem173^{KO-Lyz2}, Eif2ak3^{KO-Lyz2}, or Tmem173^{KO-Lyz2} mice. FIG. 14B shows relative expression of Ifnβ1 mRNA in different tumor-sorted myeloid populations from LLC-bearing Eif2ak3^{Flox} or Eif2ak3^{KO-Lyz2} mice. FIG. 14C shows quantitative PCR for mitochondrial Cox1 and Nd1 genes in mitochondria-free cytosolic fractions from tumor-MDSC of LLC-bearing wild type or Nfe2l2-null mice. FIG. 14D shows Ifnβ1 mRNA levels in tumor-MDSC sorted from wild type or Nfe2l2^{KO} mice bearing LLC tumors. Statistics were applied using one way ANOVA, *, p<0.05; **, p<0.01.

FIG. 15 shows FSH-CER for expression in T cells.

FIGS. 16A, 16B, 16C, and 16D show human FSH-CER T cells kill ovarian tumor cells. FIG. 16A shows FSHR expression in several ovarian primary tumors and cell line OVCAR-3. FIG. 16B shows human T cells were expanded with CD3/CD28 beads, spin infected with retronectin with hFSH-CER (2 different clones) or mock-transduced, and maintained in media with IL-7 and IL-2. At day 7, CER and control T cells were sorted on GFP expression and rested for 18 hours, before being plated with human OVCAR-3 ovarian cancer cells on the indicated effector (E) to target (T) ratios. Six hours later, luciferase signal was measured. % of specific lysis was calculated as (experimental dead−spontaneous dead)/(maximum dead−spontaneous dead)×100%. FIGS. 16C and 16D show immunohistology and tumor volume of ovarian patient-derived xenograft tumors (Wistar-3 and FCCC-0C16) grown in NSG mice (n=2 mice per tumor, one case-one control) injected i.t with 10×10⁷ FSH-CER or mock transduced T cells (arrows mark time of T-cell injection).

FIG. 17 shows FSH-CER T cells thwart progression of FSHR+ orthotopic ovarian tumors. T cells carrying FSHR-targeting CERs (FSH-CER) or identically expanded mock-transduced T cells (Mock) were administered i.p. at days 7 and 14 after orthotopic challenge with ID8-Defb29/Vegfa tumor cells expressing FSHR. Host survival was then tested. N=2 experiments (10 mice/group).

FIG. 18 shows Expression of FSHR in human ovarian carcinoma specimens and normal ovaries by W. blot (Santa Cruz #H-190).

FIGS. 19A, 19B, 19C, 19D, 19E, and 19F show Inhibition of IRE1α-XBP1 blunts tumor growth and activates effector T cell activity in tumors. FIG. 19A shows mice bearing ID8-based tumors for 7 days were treated with MKC8866 at 300 mg/kg and total peritoneal leukocytes isolated 12 h later for quantification of Xbp1s. FIG. 19B shows female mice bearing ID8-Defb29/Vegf-A tumors for 7 days were treated daily for two weeks with vehicle control or MKC8866, and peritoneal ascites samples obtained 24 h after the last treatment. Pictures denote the content of hemorrhagic ascites. FIG. 19C shows weight gain in mice from 19B. FIGS. 19D, 19E, and 19F show the frequency of T cell populations in peritoneal tumors from ID8-Defb29/Vegfa-bearing mice treated with MKC8866 for two weeks. ***P<0.0001, **P<0.001 (Student's t-test). N=two repeats with a total of 8-10 mice per group.

FIGS. 20A and 20B show Therapeutic effects of adoptive transfer with ex vivo-primed T cells devoid of XBP1. FIG. 20A shows a representative experimental scheme. FIG. 20B shows the survival of ovarian cancer-bearing mice that were left untreated (PBS, n=5) or intraperitoneally transferred with 1.5×10⁶ tumor-reactive wild-type (n=6) or XBP1-deficient T cells (n=6) at days 7 and 14 post-tumor challenge.

FIGS. 21A, 21B, 21C, 21D, and 21E show that inhibition of PERK-CHOP in T cells promotes anti-tumor activity. FIG. 21A shows tumor growth from flox and T cell-ChopKO mice bearing B16 tumors. (n=10/group). FIG. 21B shows IFNγ and TNFα in CD45+CD8+ TILs from A (n=5/group). FIG. 21C shows C57BL/6 mice (n=10/group) were injected s.c. with B16 melanoma tumors and 7 days later, received a daily i.p. dose of vehicle, GSK2606414 (25 mg/kg) or AMG-44 (12 mg/kg) until endpoint (n=10/group). FIG. 21C shows the frequency of IFNγ+CD8+ T cells in tumors from (21C) at 17 days post-tumor injection. FIG. 21E shows glucose levels in the serum of B16-bearing mice treated for a week with Perk inhibitors (day 17). *p<0.05, ***p<0.001. ns: p≥0.05.

FIGS. 22A, 22B, 22C, 22D, and 22E show Chop in recirculating T cells limits clinical responses in ovarian carcinoma. FIG. 22A shows CHOP in CD8+ TILs from ovarian carcinoma patients (OvCA Tumor) compared to peripheral blood CD8+ T cells from ovarian carcinoma patients (OvCA Blood) or healthy controls; n=15; **P<0.001. FIG. 22B shows overall survival in CD8+ CHOP^{high} (n=52) vs. CD8+ CHOP^{low} (n=29) from ovarian tumor patients (log rank 4.39, p=0.0361). FIG. 22C shows targeted heatmap using Morpheous from RNAseq values obtained from T_{RM}-T vs. recirculating T cells from ovarian tumors; n=7. FIGS. 22D and 22E show wild type and Chop^{KO} CD8+ T cells were primed with anti-CD3/CD28 and 48 hours later, T cells were tested for mitochondrial function through seahorse and mitochondrial content using qPCR of mitochondrial/nuclear DNA. n=3 repeats.

FIGS. 23A, 23B, and 23C show T cell-Chop drives tumor-induced T cell tolerance and restricts T_{RM}-T cells. FIG. 23A shows T_{RM} cells in B16 tumors injected into T cell-Chop^{KO} mice and controls (n=4). FIG. 23B shows conditioning FSH-CER T cells with PERK or IRE1α inhibitors promote expansion of T_{RM} cells in vitro. FIG. 23C shows mice bearing ID8-Defb29/Vegfa tumors for 7 days received treatment for one week with PERK or IRE1α inhibitors, followed by FSH-CER therapy on day 7 and 14 post-tumor injection (n=5).

FIG. 24 shows ovarian cancer T_{RM} CD8+ T cells show markers of enhanced effector activity and exhaustion. (Left) Heatmap of differentially expressed transcription factors (RBPJ), effector molecules (GZMB, IFNG), T_{RM} determinants (CD103/ITGAE) and exhaustion markers (PD-1/PDCD1, TIM3/HAVCR2, CTLA4 and LAGS) in CD8+ CD103+CD69+ vs. CD8+CD103− T cells FACS sorted from the same tumors. (Right) Clonality index (Adaptive Biotech) of T_{RM} vs. re-circulating CD8+ T cells from 8 different freshly dissociated human serous ovarian carcinomas (stage III/IV). Gated on (viable) cells. *P<0.05, Mann-Whitney).

FIG. 25 shows XBP1 drives PGE2 in tumor-DCs (tDC). tDCs from mice bearing ID8-Defb291 Vegfa tumors were primed with PMA/ionomycin (3 h) and PGE₂ was quantified in supernatants via ELISA. (n=5 experiments).

FIG. 26 shows PD-1 in proliferating (Ki67+) intra-tumoral CD8+CD44+ effector T cells from mice treated with vehicle or MKC8866. Data are representative from 2 repeats with a total of 8-10 mice per group. ****P<0.00001

FIG. 27 shows mRNA levels of master regulators of T_{RM} function and expansion (Fabp5, Pparg, and CD36) in flox and Chop^{KO} T cells activated with anti-CD3/28.

FIGS. 28A and 28B show FSH-CER HDR construct used to replace XBP1. FIG. 28A shows 1.3 kb DNA sequence encoding FSH-CER is spanned by 2 arms of ˜300 bp, to drive XBP1 elimination. FIG. 28B shows the feasibility study showing CRISPR-based deletion of XBP1 (left; clone #EPR22004) and expression of FSH (right; hCGa clone #381012) in primary human T cells by FACS analysis.

FIG. 29 shows a schematic of signaling pathway of PERK and IRE1α.

FIGS. 30A, 30B, 30C, and 30D show sXBP1 and Chop were evaluated by flow cytometry in viable peripheral blood CD3+CD8+ T cells collected day 7-12 after CAR T infusion, the most likely time of maximal concentration. FIG. 30A shows the % of CD8+ CAR T cells expressing sXBP1 was higher in non-responder patients than responder patients. FIG. 30A shows the % of CD8+ CAR T cells expressing Chop was not associated with response, however there was a lower % of CD8+ non CAR T cells expressing Chop in non-responders. FIG. 30C shows MFI of sXBP1 was higher in non-responding patients' CD8 CAR T cells and non CAR T cells as compared to responders. FIG. 30D shows MFI of Chop was not associated with response.

FIGS. 31A, 31B, 31C, and 31D show that sXBP1 and Chop were evaluated by flow cytometry in viable peripheral blood CD3+CD8+ T cells collected day 7-12 after CAR T infusion, the most likely time of maximal concentration. FIG. 31A shows the % of CD8+ CAR T and CD8 non-CAR T cells expressing sXBP1 was not associated with CRS. FIG. 31A shows the % of CD8+ CAR T and CD8 non-CAR T cells expressing Chop was not associated with CRS. FIG. 31C shows MFI of sXBP1 was not associated with CRS. FIG. 31D shows MFI of Chop was not associated with CRS.

FIGS. 32A, 32B, 32C, and 32D show sXBP1 and Chop were evaluated by flow cytometry in viable peripheral blood CD3+CD8+ T cells collected day 7-12 after CAR T infusion, the most likely time of maximal concentration. FIG. 32A shows the % of CD8+ CAR T and CD8 non-CAR T cells expressing sXBP1 was not associated with NT. FIG. 32B shows the % of CD8+ CAR T and CD8 non-CAR T cells expressing Chop was not associated with NT. FIG. 32C shows MFI of sXBP1 increased in both CD8+ CAR and CD8+ non-CAR T cells in patients experiencing NT. FIG. 32D shows the MFI of Chop was not associated with NT.

FIGS. 33A, 33B, 33C, and 33D show sXBP1 and Chop were evaluated by flow cytometry in CD8+ CAR T cells collected from the product bag from seven axicabtagene ciloleucel treated DLBCL patients. Cells were stimulated with tumor cell line targets, either those transduced to express CD19 target (3T3 CD19) or control without (3T3 null), for 24 hours then analyzed. FIG. 33A shows both the increase and absolute % of CD8+ CAR T cells expressing sXBP1 were higher in the non-responder patients as compared to responders. FIG. 33B shows the non-responder patients had a high % of CD8+ CAR T cells expressing Chop, both with and without CD19 stimulation. FIG. 33C shows MFI of sXBP1 was higher in the non-responding patient's CD8 CAR T cells upon stimulation whereas responders had no change compared to control. FIG. 33D shows MFI of Chop was higher in the non-responders as compared to controls, while two responding patients had no increase and one responder did show an increase compared to control.

FIGS. 34A, 34B, 34C, and 34D show sXBP1 and Chop were evaluated by flow cytometry in CD8+ CAR T cells collected from the product bag from seven axicabtagene ciloleucel treated DLBCL patients. Cells were stimulated with tumor cell line targets, either those transduced to express CD19 target (3T3 CD19) or control without (3T3 null), for 24 hours then analyzed. FIG. 34A shows the percent of CD8+ CAR T cells expressing sXBP1 when stimulated with CD19 target was increased in responders compared to non-responders. FIG. 34B shows that no significant differences in Chop as a percent of CD8+ CAR T cells were shown. FIG. 24C shows that no significant differences in sXBP1 MFI of CD8+ CAR T cells were shown. FIG. 34D shows that no significant differences in Chop MFI of CD8+ CAR T cells were shown. Non-responder is defined as those not achieving or not remaining in remission at day +90; responder is defined as those achieving and not progressing prior to day +90

FIG. 35 shows the expression profile of sXBP1 and Chop in responders and non-responders. Expression is shown for endogenous CD8+ T cells (CD8+ CAR−) and CAR T cells (CD8+ CAR+)

FIG. 36 shows XBP1 expression in total T cells and CAR T cells in for 3 patients (1 non responder, CAR51, and 2 responders, CAR 64 and 65) at various time points following administration.

FIG. 37 shows % Chop expression in total T cells and CAR T cells in for 3 patients (1 non responder, CAR51, and 2 responders, CAR 64 and 65) at various time points following administration.

FIGS. 38A, 38B, 38C, and 38D show that PERK ablation in melanoma cells induces immunogenic cancer cell death after chemically-induced ER stress. FIG. 38A shows B16-F10 and SM1 melanoma cells were transduced with Crispr-Cas 9 coding vectors targeting the expression of PERK or a Scramble (sc) control. After selection of specific clones, PERK expression was detected by immunoblot. Representative finding of N=3. FIGS. 38B, 38C, and 38D show cells from (38A) were treated with the ER stress inducer Thapsigargin and monitored for the levels of Apoptosis marker Annexin V (38B), as well as the immunogenic cell death mediators, cellular membrane translocated Calreticulin (ExoCRT), extracellular ATP, expression of IFNb mRNA (38C), and accumulation of extracellular HMGB1 (38D). Experiments were independently repeated at least 4 times.

FIGS. 39A, 39B, 39C, 39D, and 39E show that deletion of PERK in melanoma cells impairs tumor growth and elicits immunogenic cancer cell death and sustained protective T cell immunity. FIG. 39A shows wild type (WT), Scramble, or PERK-deficient (PERK^{KO}) B16-F10 or SM1 melanoma cells were implanted s.c. into C57BL/6 mice (at least N=10/group) and tumor volume evaluated daily using calipers. FIG. 39B shows C57BL/6 mice bearing SM1 tumors for 12 days received treatments with PERK inhibitor AMG-44 (12 mg/kg, i.p., every other day), followed by assessment of tumor progression (N=10). FIG. 39C shows tumor cell suspensions from mice bearing Scramble or B16-F10 tumors for 15 days were evaluated for the expression of the immunogenic cell death mediators, cellular membrane translocated Calreticulin (ExoCRT), extracellular ATP, and IFNb mRNA. FIG. 39D shows wild type (WT), Scramble, or PERK^{KO} SM1 melanoma cells were implanted into immunodeficient Rag2^{KO} mice and monitored for tumor growth every day using calipers. FIG. 39E shows naïve C57BL/6 mice and C57BL/6 mice that had previously rejected PERK^{KO}-SM1 tumors were challenged in the opposite flank with LLC or WT-SM1 tumor cells. Next, mice were followed for tumor volume. Results show that mice that had previously rejected PERK^{KO}-SM1 tumors were entirely resistant to the WT-SM1 tumor challenge, but not to LLC injection, indicating the induction specific and persistent immune memory against the primary tumor. Experiments were independently repeated at least 3 times.

FIGS. 40A and 40B show that elimination of PERK in melanoma cells induces abscopal anti-tumor effects that protect regrow of tumors. FIG. 40A shows C57BL/6 mice were primarily injected s.c. with Wild type (WT, Group 1 and 2) or PERK-deficient (PERK^{KO}, Group 3 and 4) SM1 tumors. Ten days later, mice received a secondary injection in the opposite flank of WT (Group 1 and 4) or PERK^{KO} (Group 2 and 3) SM1 tumors. FIG. 40B shows growth of the primary and secondary tumors was monitored using calipers. Results showed the development of abscopal anti-tumor events in mice that received primary PERK^{KO} tumors against WT counterparts located in the opposite flank. Conversely, no abscopal effect was observed in mice bearing primary WT-SM1 tumors.

FIGS. 41A and 41B show that ablation of PERK in melanoma cells induces protective T cell immunity and reprograms the immunosuppressive myelopoiesis into development of Monocytic-derived DCs. FIG. 41A shows C57BL/6 mice were injected s.c. with Wild type (WT), Scramble, or PERK-deficient (PERK^{KO}) B16-F10 tumors. Fifteen days later, tumors were collected and monitored via FACs for the frequency of CD8⁺ T cells (in CD45⁺ cells); recently activated CD44⁺ CD69⁺ T cells (in CD8⁺ cells); polyfunctional IFNg⁺ TNFa⁺ T cells (in CD8⁺ cells); and melanoma gp100 tetramer⁺ specific T cells (in CD8⁺ cells) (41A). Results indicate the increased expansion of T cells, recently activated T cells, effector T cells and tumor-specific T cells in PERK^{KO} B16-F10 tumors, as compared to Scramble and WT counterparts. FIG. 41B shows tumor suspensions were additionally assessed for the presence of MDSC (CD11b⁺ Gr1⁺ in CD45⁺ cells); Dendritic cells (CD11c⁺ MHC-II⁺ in CD45⁺ cells), Monocytic derived Dendritic cells (MoDCs, CD45⁺ CD11b⁺→CD11c⁺ MHC-II⁺→CD103⁺ Ly6C⁺); DC1s (CD45⁺ CD11b^{-→CD}11c⁺ MHC-II⁺→CD103⁺ Ly6C⁻); and DC2s (CD45⁺ CD11b⁺→CD11c⁺ MHCII⁺→CD103⁻ Ly6C⁺). We found a lower accumulation of MDSCs in PERK^{KO} tumors, which correlated with a higher accumulation of MoDCs.

FIGS. 42A, 42B, 42C, 42D, 42E, and 42F show PERK elimination in melanoma cells induces the expansion of cKit⁺ Ly6c⁺ myeloid precursors that differentiate into Monocytic-derived DCs (MoDCs). FIG. 42A shows C57BL/6 mice were injected s.c. with Scramble (Scr) or PERK-deficient (PERK^{KO}) B16-F10 tumors. Fifteen days later, the frequency of cKit⁺ Ly6c⁺ Myeloid Precursors (in CD45⁺) was monitored via FACs in tumors (left) and spleen (right). In addition, we evaluated the expansion in tumors of DCs (CD11c⁺ MHC-II⁺ in cKit⁺ Ly6c⁺ cells) (42B) and MoDCs (CD103⁺ in CD11c⁺ MHC-II⁺ cKit⁺ Ly6c⁺ cells) (42C). Results show the accumulation of MoDCs among the cKit⁺ Ly6c⁺ Precursors. FIG. 42D shows that to determine whether the cKit⁺ Ly6c⁺ cells serve as the precursors for the detected MoDCs in PERK^{KO} B16-F10 tumors, cKit⁺ cells were isolated from spleens of mice bearing Scr or PERK^{KO} B16-F10 tumors, cultured for 2 days in the presence of GM-CSF, and monitored for the expansion of DCs (42E) and MoDCs (42F). Results indicate the heightened expansion of MoDCs from splenic myeloid precursors isolated from PERK^{KO} B16-F10 tumors, compared to those harvested from Scr controls, suggesting the expansion of a highly immunostimulatory subset of myeloid precursors after elimination of PERK in tumors. Merged results are from 3 distinct repeats.

FIGS. 43A, 43B, 43C, and 43D show type I IFN signaling mediates the anti-tumor effects induced by the ablation of PERK in melanoma cells. FIG. 43A shows C57BL/6 and type I interferon receptor knockout (IFNAR1^{KO}) mice were injected s.c. with Scramble (Sc) or PERK-deficient (PERK^{KO}) SM1 tumors and followed for tumor progression (N=10). FIG. 43B shows C57BL/6 mice bearing Sc or PERK^{KO} B16-F10 tumors were treated with anti-IFNAR1 blocking antibodies and followed for tumor growth. Results show that elimination of IFNAR1 in the hosts or blockade of IFNAR1 overcomes the anti-tumor effects induced by the deletion of PERK in tumor cells, suggesting a key role of host-derived IFNAR1 in the anti-tumor effects induced by PERK deletion in tumors. FIG. 43C shows elimination of IFNAR1 in the hosts or ab-based IFNAR1 blockade prevents the tumor accumulation of myeloid cKit⁺ Ly6c⁺ precursors induced by the ablation of PERK in B16-F10 tumor cells. FIG. 43D shows expansion of DCs and MoDCs in WT and IFNAR1^{KO} mice carrying Sc or PERK^{KO} B16-F10 tumors. Similar to the delayed expansion of myeloid precursors (C), deletion of IFNAR1 in the hosts overcomes the accumulation of DC and MoDCs induced by the elimination of PERK in the tumor cell compartment.

FIGS. 44A, 44B, and 44C show that Type I IFNs-dependent CCR2 controls the infiltration of immune stimulatory myeloid precursors in PERK^{KO} tumors. FIG. 44A shows CCR2 expression in tumor-related DCs (CD11b⁺ MHC-II⁺) from Scramble (Sc) or PERK-deficient (PERK^{KO}) B16-F10 tumors injected into C57BL/6 or type I interferon receptor knockout (IFNAR1^{KO}) mice (N=4). Results show that deletion of IFNAR1 prevents the expression of CCR2 in DCs expanding in PERK^{KO} tumors. FIG. 44B shows C57BL/6 and CCR2^{KO} mice were injected with Sc or PERK^{KO} B16-F10 tumors and followed for tumor progression. Results show that elimination of CCR2 in mice blunts the anti-tumor effects observed in PERK^{KO} tumors. FIG. 44C shows elimination of CCR2 in mice overcomes the expansion of Myeloid Precursors (cKit⁺ Ly6c⁺) and MoDCs induced by the elimination of PERK in B16-F10 tumor cells. *p<0.05, ****p<0.001. ns: p≥0.05

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“Z¹,” “Z²,” “Z³,” and “Z⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, for example 1 to 3, 1 to 4, 1 to 5, 1 to 6, 1 to 7, 1 to 8, 1 to 9, 1 to 10, or 1 to 15 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage; that is, an “alkoxy” group can be defined as —OZ¹ where Z¹ is alkyl as defined above.

The term “alkenyl” as used herein is a hydrocarbon group of from 2 to 24 carbon atoms, for example, 2 to 5, 2 to 10, 2 to 15, or 2 to 20 carbon atoms, with a structural formula containing at least one carbon-carbon double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms, for example 2 to 5, 2 to 10, 2 to 15, or 2 to 20 carbon atoms, with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl or heteroaryl group can be substituted or unsubstituted. The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “carbonyl as used herein is represented by the formula —C(O)Z¹ where Z¹ can be a hydrogen, hydroxyl, alkoxy, alkyl, halogenated alkyl, alkenyl, alkynyl, aryl, heteroaryl, cycloalkyl, cycloalkenyl, heterocycloalkyl, or heterocycloalkenyl group described above. Throughout this specification “C(O)” or “CO” is a short hand notation for C═O.

The term “aldehyde” as used herein is represented by the formula —C(O)H.

The terms “amine” or “amino” as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NZ¹Z².

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)Z¹ or —C(O)OZ¹, where Z¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula Z¹OZ², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula Z¹C(O)Z², where Z¹ and Z² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” or “halogen” as used herein refers to the fluorine, chlorine, bromine, and iodine.

The term “hydroxyl” as used herein is represented by the formula —OH.

The term “nitro” as used herein is represented by the formula —NO₂.

The term “silyl” as used herein is represented by the formula —SiZ¹Z²Z³, where Z¹, Z², and Z³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂Z¹, where Z¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

The term “thiol” as used herein is represented by the formula —SH.

The term “thio” as used herein is represented by the formula —S—.

“R¹,” “R²,” “R³,” “Re,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples and Figures.

### C. Compounds

The compounds disclosed herein are potent polycyclic IRE-1 RNase inhibitors. U.S. Pat. No. 10,323,013 is incorporated herein by reference for its disclosure of said inhibitors, methods of making the inhibitors, and methods of their use. As such, disclosed herein are compounds having Formula I:

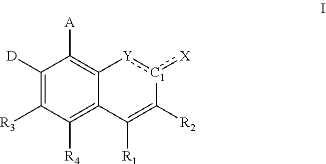

wherein
the dotted lines between Y and C₁ and C₁ and X represent single or double bonds, as valency permits;
A is a chalcogen containing moiety;
D is chosen from hydrogen, hydroxyl, carbonyl, alkoxy, halogen, thiol, thioalkyl, or alkyl;
R³ and R⁴ are independently chosen from hydrogen, halogen, hydroxy, amino, alkyl, alkenyl, alkynyl, haloalkyl, cycloalkyl, heterocycloalkyl, alkylaryl, aryl, alkylheteroaryl, or heteroaryl, any of which is optionally substituted with carbonyl, alkyl, amino, amido, —NR⁶R⁷, —C(O)NR⁶R⁷, alkoxy, alkylhydroxy, cycloalkyl, heterocycloalkyl, aryl, heteroaryl, carbonyl, halo, hydroxy, thiol, cyano, or nitro;
Y is chosen from S, N, O or C,
wherein when Y is C, the dotted line between Y and C₁ in the ring represents a double bond and the dotted line between C₁ and X is a single bond; and
wherein when Y is S, N or O, the dotted line between Y and C₁ in the ring represents a single bond and the dotted line between C₁ and X represents a double bond;
X represents, as valency permits, hydrogen, oxygen, halogen, hydroxy, amino, thiol, thioalkyl, alkyl, alkenyl, alkynyl, haloalkyl, cycloalkyl, heterocycloalkyl, alkylaryl, aryl, alkylheteroaryl, or heteroaryl, any of which is optionally substituted with acetyl, alkyl, amino, amido, —NR⁶R⁷, —C(O)NR⁶R⁷, alkoxy, alkylhydroxy, cycloalkyl, heterocycloalkyl, aryl, heteroaryl, carbonyl, halo, hydroxy, thiol, cyano, or nitro;
R¹ and R² are independently chosen from hydrogen, benzoate, alkyl, alkenyl, alkynyl, haloalkyl, cycloalkyl, heterocycloalkyl, alkylaryl, aryl, alkylheteroaryl, or heteroaryl, any of which is optionally substituted with acetyl, alkyl, amino, amido, —NR⁶R⁷, —C(O)NR⁶R⁷, alkoxy, alkylhydroxy, cycloalkyl, heterocycloalkyl, aryl, heteroaryl, carbonyl, halo, hydroxy, thiol, cyano, or nitro; or
R¹ and R² together with the atoms to which they are attached form a 5-7 membered cyclic moiety wherein any of the additional atoms can be heteroatoms and the 5 to 7-membered ring is, optionally, a heterocyclic structure that is optionally substituted; and
R⁶ and R⁷ are independently H, alkyl; or
R⁶ and R⁷ together with the atoms to which they are attached form a 3-7 membered cyclic moiety wherein any of the additional atoms can be heteroatoms and the 3 to 7-membered ring is optionally a heterocyclic structure that is optionally substituted;
   or a pharmaceutically acceptable salt or prodrug thereof.

Example chalcogen containing moieties are aldehyde, protected aldehyde (e.g., dioxane and dithiane), reduced aldehyde, benzoate, ester, ketone, carbonyl, ether, carboxylic acid, alcohol, or alkoxyl groups. Also, chalcogen containing moieties can include amine, amide, sulfonamide, sulfonyl, sulfinyl, halogenated alkyl, CH═CH—CO₂R⁶, CH═CHSO₂R⁶; where R⁶ is H, OH, or alkyl. Example benzoate groups are methyl benzoate.

In some examples of Formula I, D is OH, R³ and R⁴ are both hydrogen, Y is C and X is H, resulting in compounds of Formula II:

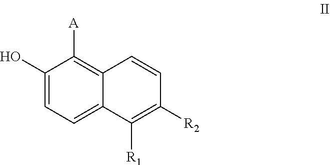

wherein A, R¹ and R² are as defined in Formula I.

In some examples of Formula I, D is OH, R³ and R⁴ are both hydrogen, Y is O and X is O, resulting in compounds of Formula III:

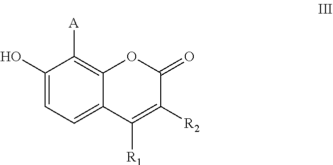

wherein A, R¹ and R² are as defined in Formula I.

In some examples of Formula III, A is an aldehyde, a protected aldehyde or a reduced aldehyde. In some examples of Formula III, R² is hydrogen and R¹ is a carbamate.

In further examples of Formula III, the disclosed compounds can have Formula III-A

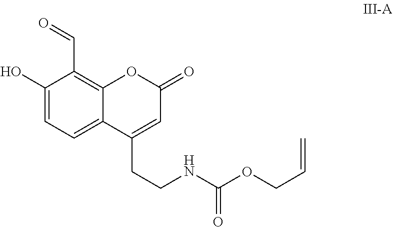 [SMILES: C=CCOC(=O)NCCc1cc(=O)oc2c(C=O)c(O)ccc12]

In some examples of Formula I, D is OH, R³ and R⁴ are both hydrogen, Y and X are both O, and R¹ and R² form a 6-membered heterocycle with nitrogen resulting in compounds of Formula IV:

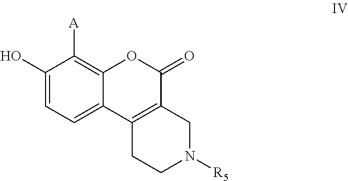

wherein
A is as defined above
R⁵ is chosen from hydrogen, benzoate, alkyl, alkenyl, alkynyl, haloalkyl, cycloalkyl, heterocycloalkyl, alkylaryl, aryl, alkylheteroaryl, or heteroaryl, any of which is optionally substituted with acetyl, alkyl, amino, amido, —NR⁶R⁷, —C(O)NR⁶R⁷, alkoxy, alkylhydroxy, cycloalkyl, heterocycloalkyl, aryl, heteroaryl, carbonyl, halo, hydroxy, thiol, cyano, or nitro; or
   a pharmaceutically acceptable salt or prodrug thereof.

In some examples of Formula IV, A is an aldehyde, a protected aldehyde or a reduced aldehyde. In some examples of Formula IV, R⁵ is an ester.

In some examples, the disclosed compounds can have Formula IV-A

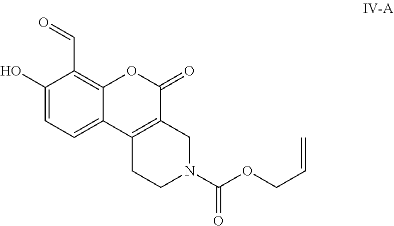 [SMILES: C=CCOC(=O)N1CCc2c(c(=O)oc3c(C=O)c(O)ccc23)C1]

In some examples, the disclosed compounds can have Formula IV-B

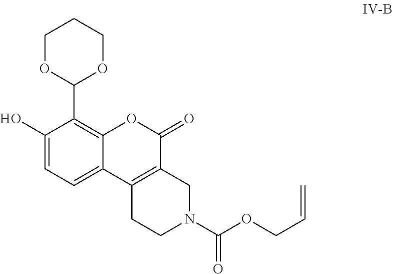 [SMILES: C=CCOC(=O)N1CCc2c(c(=O)oc3c(C4OCCCO4)c(O)ccc23)C1]

In some examples, the disclosed compounds can have Formula IV-C

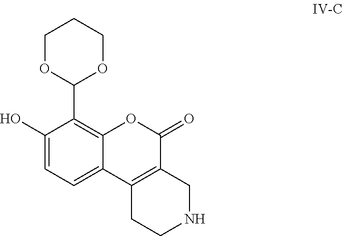 [SMILES: O=c1oc2c(C3OCCCO3)c(O)ccc2c2c1CNCC2]

In some examples of Formula I, A is an aldehyde, D is OH, R³ and R⁴ are both hydrogen, Y and X are both O, and R¹ and R² form a 6-membered heterocycle with nitrogen resulting in compounds of Formula V:

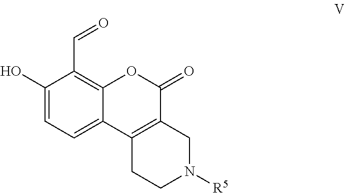

wherein
R⁵ is chosen from hydrogen, benzyl, substituted benzyl, acetate, alkyl, substituted alkyl, amidine, or substituted amindine; or a pharmaceutically acceptable salt or prodrug thereof.

In some examples of Formula I, A is an aldehyde, R³ and R⁴ are both hydrogen, Y and X are both O, and R¹ and R² form a 6-membered heterocycle with nitrogen resulting in compounds of Formula VI:

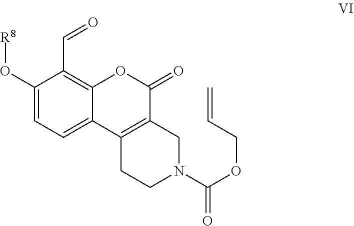

wherein
R⁸ is chosen from hydrogen, carbonyl, alkoxy, halogen, thiol, thioalkyl, aryl, alkylaryl, or alkyl; or a pharmaceutically acceptable salt or prodrug thereof.

In some examples of Formula I, D is OH, R³ and R⁴ are both hydrogen, Y and X are both O, and R¹ and R² form a 6-membered heterocycle with nitrogen resulting in compounds of Formula VII:

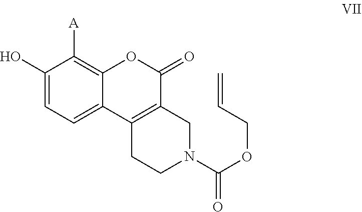

wherein
A is chosen from hydroxyl, hydroxyl, alkoxy, carboxyl, carboxylic acid, ether, ester, amine, amide, dioxane, dithiane, ketone, aldehyde, sulfonamide, sulfonyl, sulfinyl, halogenated alkyl, CH═CH-CO₂R⁶, CH═CHSO₂R⁶; where R⁶ is H, OH, or alkyl; or a pharmaceutically acceptable salt or prodrug thereof.
In any of Formulas I-VII, D can be preferably OH. Also, in any of Formulas I-VII R³ and R⁴ are preferably both H.

In some specific examples, the disclosed compounds can have any one of the following structures:

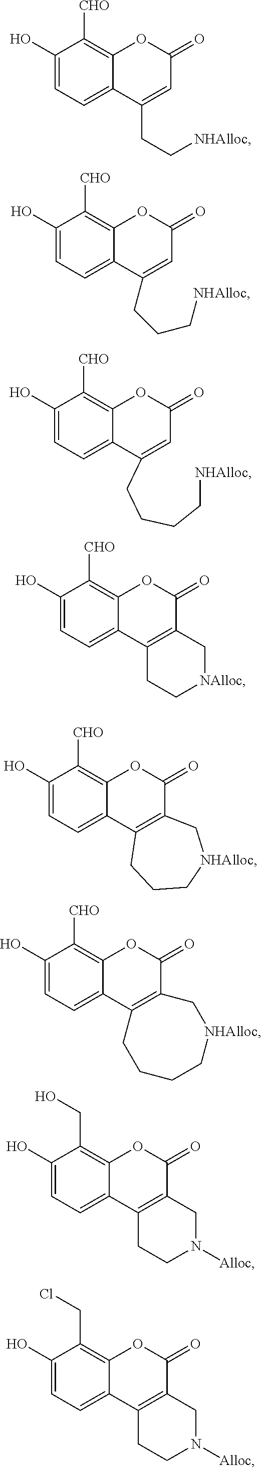

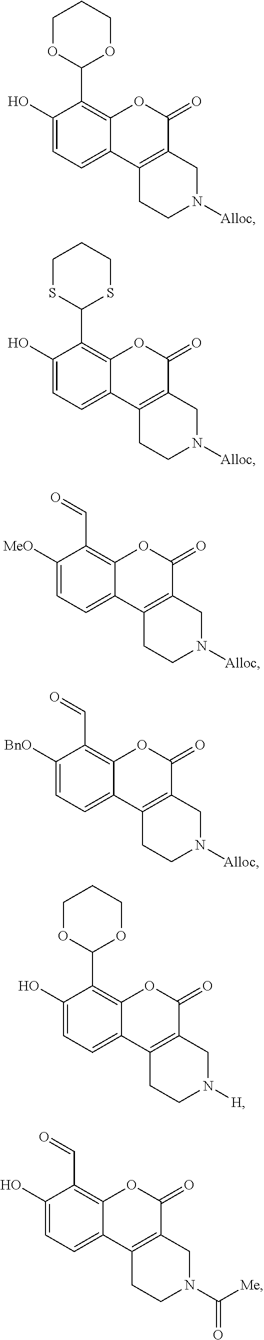

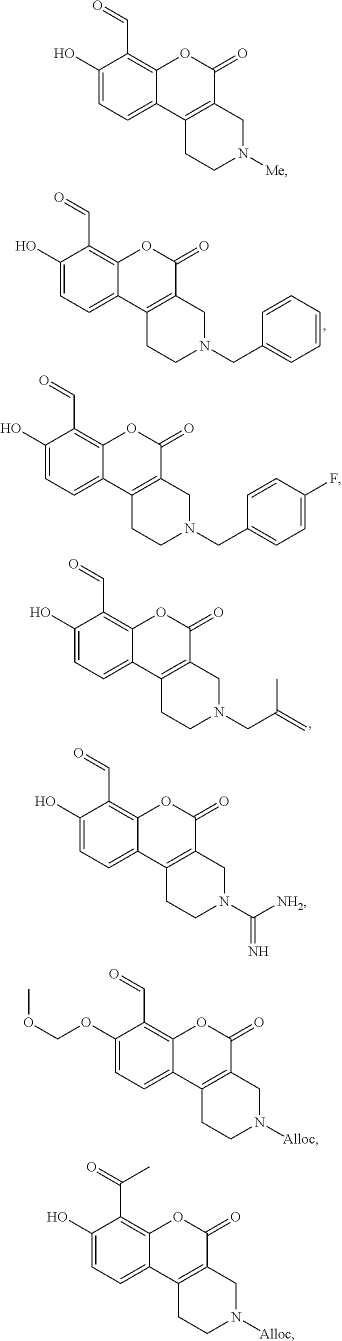

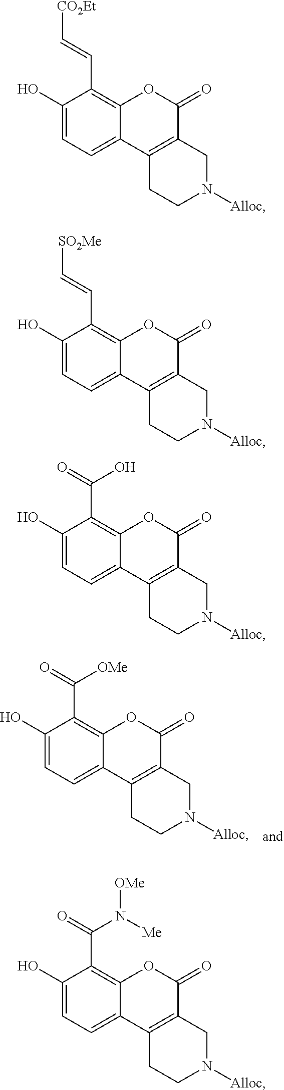

wherein Alloc is an allyloxycarbonyl moiety.

The syntheses of the compounds disclosed herein are addressed in more detail in the examples.

Also disclosed herein are pharmaceutically-acceptable salts and prodrugs of the disclosed compounds. Pharmaceutically-acceptable salts include salts of the disclosed compounds that are prepared with acids or bases, depending on the particular substituents found on the compounds. Under conditions where the compounds disclosed herein are sufficiently basic or acidic to form stable nontoxic acid or base salts, administration of the compounds as salts can be appropriate. Examples of pharmaceutically-acceptable base addition salts include sodium, potassium, calcium, ammonium, or magnesium salt. Examples of physiologically-acceptable acid addition salts include hydrochloric, hydrobromic, nitric, phosphoric, carbonic, sulphuric, and organic acids like acetic, propionic, benzoic, succinic, fumaric, mandelic, oxalic, citric, tartaric, malonic, ascorbic, alpha-ketoglutaric, alpha-glycophosphoric, maleic, tosyl acid, methanesulfonic, and the like. Thus, disclosed herein are the hydrochloride, nitrate, phosphate, carbonate, bicarbonate, sulfate, acetate, propionate, benzoate, succinate, fumarate, mandelate, oxalate, citrate, tartarate, malonate, ascorbate, alpha-ketoglutarate, alpha-glycophosphate, maleate, tosylate, and mesylate salts. Pharmaceutically acceptable salts of a compound can be obtained using standard procedures well known in the art, for example, by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

### D. Methods of Use

Further provided are methods of treating or preventing a disease or pathology in a subject, comprising administering to the subject an effective amount of a compound or composition as disclosed. The disease can be associated with the transcription factor, XBP-1 activity. The disease can also be associated with the endoplasmic reticulum-resident, IRE-1 RNase activity. In some embodiments, the disease can be associated with upregulation of the IRE-1/XBP-1 pathway.

The disclosed compounds and compositions can electively inhibit IRE-1 RNase activity. For example, the compounds can inhibit IRE-1 RNase activity with 50% inhibitory concentration (IC₅₀) values of less than about 150 nM, less than 100 nM, less than 75 nM, less than 50 nM, less than 40 nM, less than 30 nM, less than 25 nM, less than 20 nM, less than 15 nM, or less than 10 nM. In some embodiments, the disclosed compounds and compositions can selectively inhibit the expression of XBP-1. In some embodiments, the disclosed compounds and compositions can selectively inhibit Akt signaling. In some embodiments, the disclosed compounds and compositions do not target critical cellular mechanisms involved in protein transport. For example, the disclosed compounds and compositions do not target secretory protein transport. In some embodiments, the disclosed compounds and compositions suppress disease progression, for e.g., leukemia without imposing systemic toxicity.

Further provided herein are methods of treating or preventing a disease, for example cancer in a subject, comprising administering to the subject an effective amount of a composition comprising a adoptive immunotherapy (such as, for example chimeric antigen receptor (CAR) T cell (CAR T cell) immunotherapy, Tumor infiltrating lymphocyte (TIL) immunotherapy, CAR NK cell immunotherapy, and or marrow infiltrating lymphocyte (MIL) immunotherapy) and any of the compounds disclosed herein.

Methods of killing a tumor cell are also provided herein. The methods comprise contacting a tumor cell with an effective amount of a compound or composition as disclosed herein and an adoptive immunotherapy. The methods can further include administering a second compound or composition (e.g., an anticancer agent) or administering an effective amount of ionizing radiation to the subject.

As noted throughout this application, it is understood and herein contemplated that the methods and inhibitors disclosed herein (such as, for example, any of the a PERK inhibitors, ER Stress inhibitors, and/or an IRE1 inhibitors disclosed herein) alone or in combination with adoptive immunotherapies (such as, for example, CAR T cell, CAR NK cell, TIL, and MIL immunotherapies) can be used to treat, inhibit, reduce, ameliorate, and/or prevent any disease where uncontrolled cellular proliferation occurs such as cancers (including, but not limited to primary cancers and metastasis). A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

It is understood and herein contemplated that the administration of a PERK inhibitor, ER Stress inhibitor, and/or an IRE1 inhibitor has a therapeutic effect on a cancer and can reduce, inhibit, decrease, ameliorate or kill the cancer and/or metastasis without further intervention. Thus, in one aspect, disclosed herein are methods of treating, inhibiting, reducing, decreasing, ameliorating, and/or preventing a cancer and/or metastasis in a subject comprising administering to the subject a PERK inhibitors, ER Stress inhibitors, and/or an IRE1 inhibitor.

As shown herein that a PERK inhibitor, ER Stress inhibitor, and/or an IRE1 inhibitor alone can treat cancer. Nevertheless, the methods can benefit by the further addition of adoptive immunotherapies (such as, for example, CAR T cell, CAR NK cell, TIL, and MIL immunotherapies). Thus, as noted above, disclosed herein are methods of treating, inhibiting, reducing, decreasing, ameliorating, and/or preventing a cancer and/or metastasis in a subject comprising administering to the subject a PERK inhibitors, ER Stress inhibitors, and/or an IRE1 inhibitor; further comprising the administration of a adoptive immunotherapies (such as, for example, CAR T cell, CAR NK cell, TIL, and MIL immunotherapies). Given the ability that the disclosed PERK inhibitors, ER Stress inhibitors, and/or an IRE1 inhibitor have on T cells, CAR T cells, and CAR NK cells, it is understood that the T cells, TILs, MILs, CAR T cells, and/or CAR NK cells can be contacted with PERK inhibitor(s), ER Stress inhibitor(s), and/or an IRE1 inhibitor(s) ex vivo prior to administration. This effect of the PERK inhibitor(s), ER Stress inhibitor(s), and/or an IRE1 inhibitor(s) is to result in better killing by the T cells, TILs, MILs, CAR T cells, and/or CAR NK cells.

In one aspect, it is understood and herein contemplated that successful treatment of a cancer in a subject is important and doing so may include the administration of additional treatments. Thus, the disclosed treatments can further include any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Where an EGFR splice variant isoform is not detected, the treatment methods can include or further include checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### E. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Unfolded Protein Response Sensor PERK Governs Myeloid Cell-Driven Immunosuppression in Tumors Through NRF2-Dependent Inhibition of STING Signaling

The myelopoiesis process that protects against tumors is drastically derailed in most individuals with advanced malignancies towards the expansion of myeloid subsets that block protective anti-tumor T cell immunity and promote cancer cell progression and metastasis (Singhal et al., 2016). Expansion of myeloid-derived suppressor cells (MDSC), a heterogeneous group of monocytic (M-MDSC) and polymorphonuclear (PMN-MDSC) precursors, has emerged as a key mechanism of anti-tumor immune evasion (Veglia et al., 2018) and correlates with poor clinical outcome and resistance to cancer immunotherapy (Glodde et al., 2017; Lu et al., 2017; Weber et al., 2016). Upon infiltration into the tumor microenvironment (TME), MDSC amplify their immunoregulatory potential through the upregulation of Arginase I and Nitric oxide synthase 2 (NOS2), the release of reactive oxygen species (ROS) and peroxynitrite (PNT), and the production of several regulatory cytokines (Gabrilovich et al., 2012; Kumar et al., 2016). Despite their relevance in tumors, there are limited strategies to therapeutically inhibit the detrimental activity of MDSC in patients with cancer, in part because of the incomplete understanding of the primary mechanisms governing the MDSC functions and plasticity within the TME.

Multiple cytokines, growth factors, and stress-related conditions in the TME polarize tumor-infiltrating MDSC into highly immunosuppressive cells. However, the mechanisms by which MDSC evolve to augment their immunosuppressive potential in the complex and harsh tumor milieu remain elusive. Activation of the unfolded protein response (UPR) controls the quality of the proteome pool in cells experiencing accumulation of misfolded proteins in the endoplasmic reticulum (ER), which leads to ER stress. Priming of the UPR also functions as a physiological signaling program in differentiating cells undergoing ER expansion as the result of the high secretory and metabolic activity triggered by cytokines, Toll-like receptors agonists, or other stimuli. The UPR is characterized by the orchestrated stimulation of the activating transcription factor-6 (ATF6), the inositol-requiring enzyme 1 (IRE1α), and the protein kinase RNA (PKR)-like ER kinase (PERK, encoded by the Eif2ak3 gene) (Hetz and Papa, 2018; Walter and Ron, 2011). Activation of PERK occurs through oligomerization and auto-phosphorylation, leading to the phosphorylation of multiple targets, including the transcription factor NF-E2-related factor 2 (NRF2, encoded by the Nfe2l2 gene) (Pytel et al., 2016). In tumor cells, PERK-linked phosphorylation of NRF2 induces cellular redox transcripts that relieve the effects of ROS (Cullinan and Diehl, 2004; Cullinan et al., 2003). Although stimulation of the UPR drivers modulates the survival of cancer cells in the TME (Chevet et al., 2015; Urra et al., 2016), their role in the immunoinhibitory activity of myeloid cells in tumors is incompletely understood. Seminal reports highlighted the impact of IRE1α-Xbp1 and the unresolved ER stress sensor C/EBP homologous protein (CHOP, encoded by Ddit3 gene) in the immunoregulatory activity of myeloid cells in tumors (Condamine et al., 2016; Cubillos-Ruiz et al., 2015; Shang et al., 2017; Thevenot et al., 2014; Yan et al., 2016). However, the mechanisms whereby the activation of the UPR, and in particular PERK signaling, controls the immunoregulatory activity of MDSC in tumors remain unknown.

The stimulator of interferon genes (STING, encoded by Tmem173 gene) represents a major mediator of innate immune responses triggered by DNA (Ishikawa and Barber, 2008; Wu et al., 2013). Primed STING recruits and promotes the phosphorylation of the TANK-binding kinase 1 (TBK1), which phosphorylates the interferon regulatory factor 3 (IRF3) (Tanaka and Chen, 2012; Zhang et al., 2019), thereby promoting the production of type I interferons (IFNs) (Ahn et al., 2014). Although STING activation in myeloid cells has emerged as a key driver of anti-tumor immunity (Woo et al., 2014), the pathways regulating the intrinsic activation of STING signaling in tumor-infiltrating MDSC continue to be poorly understood.

Here, we sought to elucidate the role of PERK in the immunosuppressive activity of tumor-MDSC. Elevated PERK activation was detected in tumor-infiltrating MDSC from mice and cancer patients. Deletion of PERK re-routed MDSC into cells that primed anti-tumor CD8⁺ T cell immunity by reducing NRF2 signaling, which thereby impaired mitochondrial homeostasis and triggered STING-dependent production of Type I IFNs through accumulating cytosolic mitochondrial DNA (mtDNA). Our results demonstrate the mechanistic interaction between PERK, NRF2, and STING in the function of tumor-MDSC, and suggest the targeting of PERK as a strategy to resolve immunosuppressive myelopoiesis in tumor-bearing hosts and to enhance the effects of cancer immunotherapy.

a) Results

(1) TME Promotes Immunoregulatory Activity and Activation of the UPR in MDSC

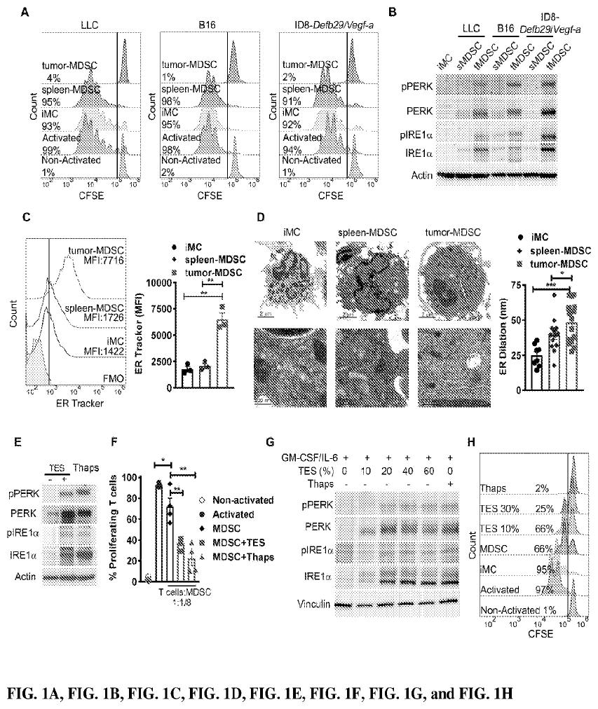

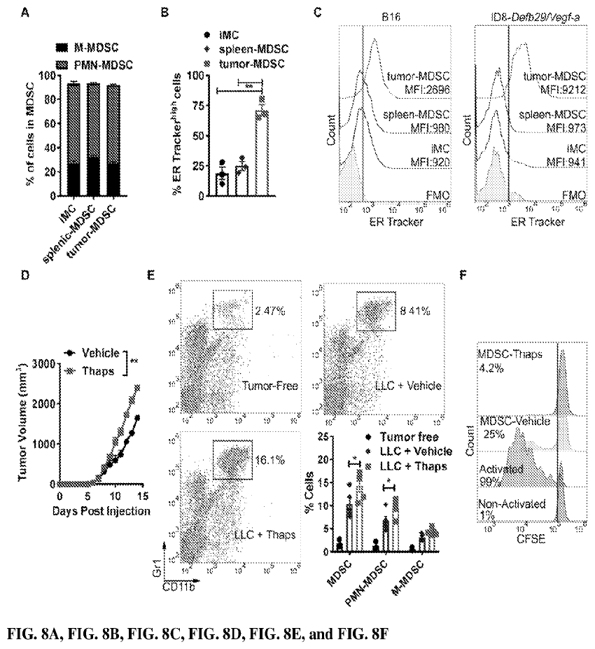

We tested the crosstalk between the T cell inhibitory potential and the activation of UPR sensors in CD11b⁺ Gr1⁺ cells from tumors (tumor-MDSC) and spleens (spleen-MDSC) of mice bearing s.c. Lewis lung carcinoma (LLC), s.c. B16 melanoma, or orthotopic ID8-Defb29/Vegf-a ovarian tumors; and in splenic myeloid counterparts (iMC) from tumor-free mice. A superior capacity to impair T cell proliferation and an increased expression of both total and phosphorylated PERK and IRE 1α were detected in tumor-MDSC compared to splenic-MDSC or iMC (FIG. 1A-B), without a distinct of accumulation of MDSC subsets (FIG. 8A). In addition, consistent with the preferential expansion of the ER in tumor-MDSC, elevated fluorescence of ER tracker, a dye that binds to the sulphonyl-urea receptors in the ER ATP-sensitive K⁺ channels (Hambrock et al., 2002) (FIG. 1C, FIG. 8B-C), and augmented ER dilation detected by transmission electron microscopy (FIG. 1D) were found in tumor-MDSC compared to splenic-MDSC or iMC. Next, we tested whether the exposure of bone marrow-derived MDSC (BM-MDSC) to tumor explants (TES) promoted activation of the UPR sensors and immunoregulatory potential. Pre-treatment of murine BM-MDSC with primary LLC-TES (Thevenot et al., 2014), or human BM-MDSC with supernatants from renal cell carcinoma cell line 786-0 (hTES) (Rodriguez et al., 2009), resulted in upregulation of total and phosphorylated PERK and IRE1α and enhanced ability to block T cell proliferation, which were similar to the effects triggered by the ER-calcium depleting agent and UPR inducer, Thapsigargin (Thaps) (FIG. 1E-H). To identify the effect of Thaps in tumor growth and MDSC activity, we then treated LLC-bearing mice with Thaps. Thaps administration promoted tumor progression, increased the expansion of splenic-MDSC, and elevated the immunoregulatory potential of tumor-MDSC (FIG. 8D-F). Together, these results suggest a link between the UPR activation and the immunoinhibitory potential in TME-exposed MDSC.

(2) Mitigation of the UPR Activation Blunts MDSC Activity and Boosts Cancer Immunotherapy

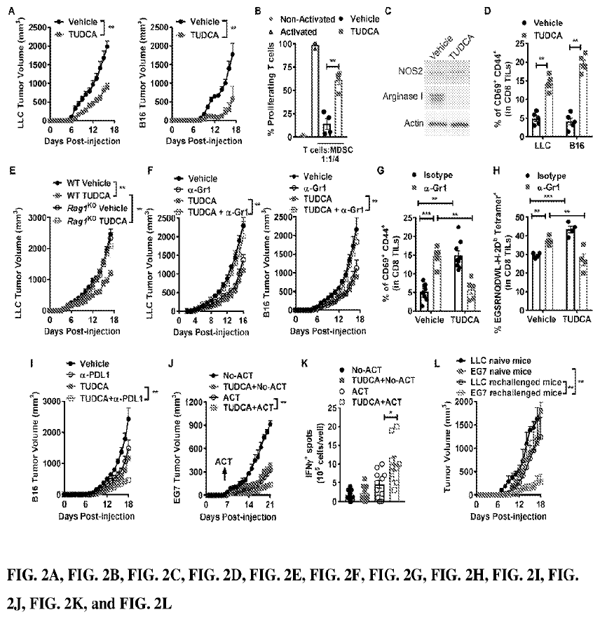

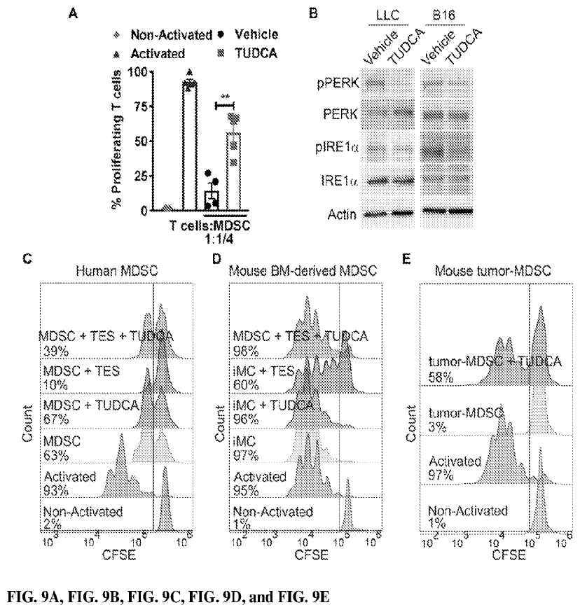

We evaluated the anti-tumor effects of Tauroursodeoxycholic acid (TUDCA), a molecular chaperone previously reported to alleviate the effects induced after ER stress and UPR activation (Nakagawa et al., 2014; Ozcan et al., 2006). Treatment of mice bearing established LLC or B16 tumors with TUDCA delayed tumor growth (FIG. 2A). In addition, lower ability to block T cell proliferation (FIG. 2B, FIG. 9A), and reduced levels of the inhibitory factor Arginase I, but not NOS2 (FIG. 2C), were detected in tumor-MDSC from mice treated with TUDCA, compared to counterparts from vehicle-treated mice. In support to the mitiagation of the UPR by TUDCA, lower expression of phospho-PERK and phospho-IRE 1α was noticed in tumor-MDSC from mice treated with TUDCA, compared to controls (FIG. 9B). Because the activity of MDSC depends on tumor burden, we then evaluated the effect of TUDCA in cultured MDSC. TUDCA impaired the ability of human BM-MDSC conditioned with hTES, murine BM-MDSC developed after exposing BM-iMC to TES, and LLC tumor-MDSC to block T cell proliferation (FIG. 9C-E). Next, we sought to establish the role of T cells in the anti-tumor effects induced by TUDCA. Higher expansion of recently activated CD69⁺ CD44⁺ CD8⁺ T cells was detected in tumors from B16 or LLC-bearing mice treated with TUDCA (FIG. 2D). Also, the anti-tumor actions triggered by TUDCA in wild type mice were not found in immunodeficient Rag1^{KO} mice (FIG. 2E), suggesting the role of T lymphocytes in the anti-tumor effects induced by TUDCA. Next, we studied whether treatment of tumor-bearing mice with TUDCA switched MDSC into cells that elicited anti-tumor responses. Depletion of MDSC-like cells upon continuous injections with anti-Gr1 antibody (Sierra et al., 2017) delayed B16 and LLC tumor progression in vehicle-treated control mice, while restoring tumor growth in TUDCA-treated counterparts (FIG. 2F). Notably, anti-Gr1 or TUDCA treatments promoted the spontaneous expansion of recently activated CD69⁺ CD44⁺ CD8⁺ T cells and EGSRNQDWL-H-2D^{b}-tetramer⁺ CD8⁺ T cells recognizing the gp100 melanoma antigen on B16-bearing mice (FIG. 2G-H). In contrast, the accumulation of these tumor-reactive T cell groups was prevented in TUDCA-treated mice after co-administration with anti-Gr1 (FIG. 2G-H). These results indicate that TUDCA drives the functional transformation of tumor-MDSC into T cell-stimulating populations.

Furthermore, we tested whether TUDCA amplified the therapeutic effect of anti-PD-L1 blockade immunotherapy. Treatment with TUDCA plus anti-PD-L1 induced synergistic anti-tumor effects in B16-bearing mice, compared to mice treated with the single agents (FIG. 2I). Next, we evaluated the effect of TUDCA in an adoptive T cell transfer (ACT) model against the experimental antigen ovalbumin (OVA), in which pre-activated anti-OVA257-264 (SIINFEKL) OT-I cells were transferred into mice bearing OVA⁺ EG7 tumors Amplified anti-tumor response was noticed in mice treated with TUDCA plus ACT, compared to those receiving TUDCA or ACT single treatments or untreated controls (FIG. 2J). Accordingly, EG7-bearing mice treated with TUDCA plus ACT had higher frequency of IFNγ-producing T cells upon ex vivo activation of splenocytes with SIINFEKL (FIG. 2K). Notably, some of the mice treated with TUDCA plus ACT rejected the EG7 tumors and were resistant to re-challenge with the same tumor cells, but not against non-related tumors (FIG. 2L), indicative of specific anti-tumor immunological memory. These results demonstrate the potential of TUDCA to overcome MDSC-linked T cell dysfunction and to boost the effect of cancer immunotherapy in tumor-bearing mice.

(3) PERK Regulates Immunosuppressive Activity of Tumor-MDSC

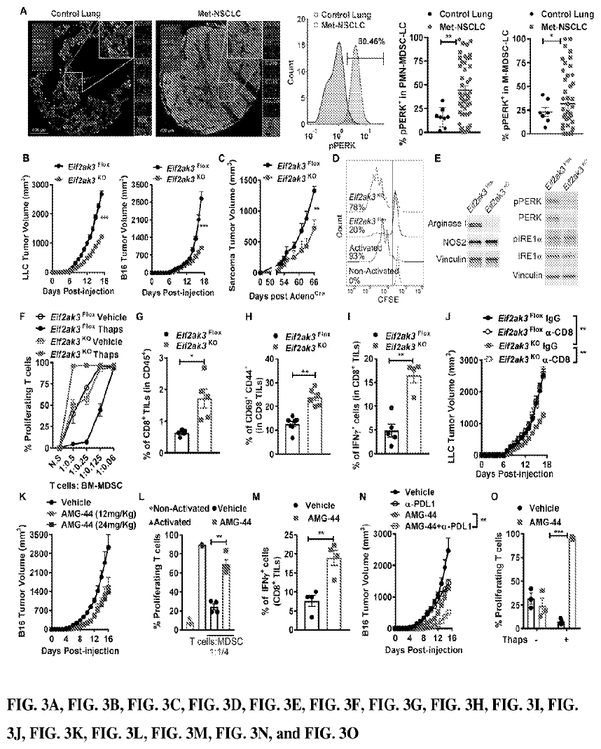

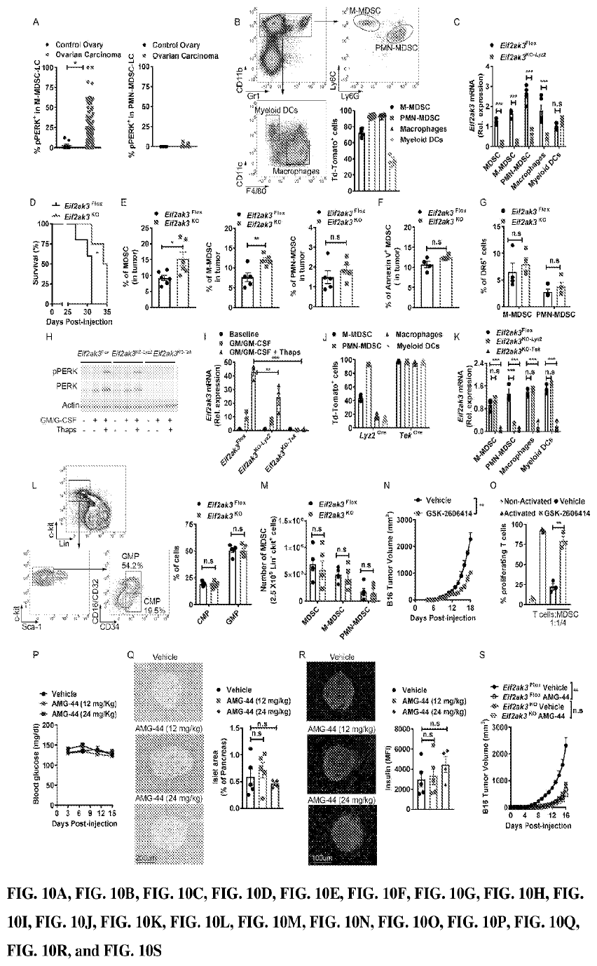

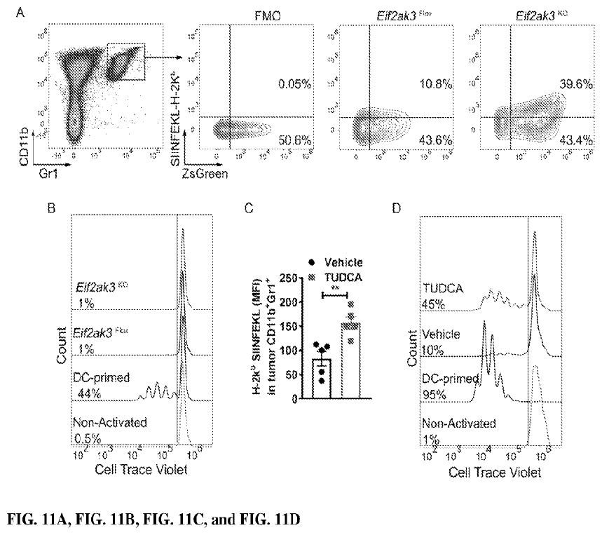

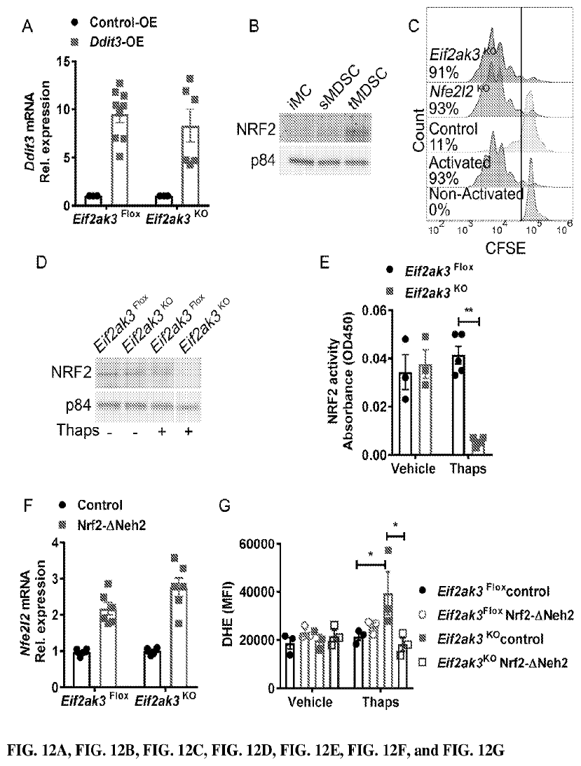

We next monitored the expression of phospho-PERK in tumor-MDSC from a TMA created from 46 patients with metastatic non-small cell lung carcinoma (Met-NSCLC) through high-resolution automated multispectral imaging. Heightened phospho-PERK levels were detected in cells resembling PMN-MDSC (Pan-Cytokeratin^{neg}CD11b⁺HLA-DR^{neg} CD14^{neg} CD15⁺) and M-MDSC (Pan-Cytokeratin^{neg}CD11b⁺HLA-DR^{neg}CD14⁺CD15^{neg}) from Met-NSCLC tumors, compared to the cellular counterparts from healthy lung tissues (FIG. 3A). In addition, we validated the expression of phospho-PERK in MDSC from a TMA from 84 patients with advanced high-grade serous ovarian cancer. Elevated frequency of phospho-PERK⁺ M-MDSC was found in ovarian tumors, compared to similar cellular subsets from healthy ovaries (FIG. 10A). Interestingly, we could not consistently analyze the levels of phospho-PERK in PMN-MDSC from ovarian tumors as limited infiltration of cells resembling PMN-MDSC was noted (FIG. 10A). Together, results show the elevated PERK activation in tumor-MDSC from different human malignancies.

To study the impact of PERK in MDSC on tumor growth, we generated myeloid cell-conditional Eif2ak3 null mice, after breeding Eif2ak3^{Flox} mice with mice expressing lysozyme (Lyz2)-driven Cre recombinase (Eif2ak3^{KO-Lyz2}), which enabled gene excision in tumor-infiltrating M-MDSC, PMN-MDSC, and macrophages, and to a lower extent in myeloid DCs (FIG. 10B-C). Delayed growth of s.c. tumors LLC and B16, and prolonged survival after orthotopic injection with ID8-Defb29/Vegf-a ovarian tumors, were observed in Eif2ak3^{KO-Lyz2} mice compared to Eif2ak3^{Flox} controls (FIG. 3B, 10D). To expand our data in a spontaneous tumor model, lethally-irradiated LSL-K-RasG^{12D+}-Trp53^{fl/fl} mice received bone marrow cells from Eif2ak3^{KO-Lyz2} or Eif2ak3^{Flox} mice. Seven weeks later, the mice were injected with intra-muscular adenovirus coding for Cre-recombinase and followed for growth of autochthonous flank sarcomas, which depends on MDSC mobilization and activity (Rutkowski et al., 2015). Similar to the transplantable models, lower tumor progression was noticed in LSL-K-Ras^{G12D+}-Trp53^{KO} mice reconstituted with bone marrows from Eif2ak3^{KO-Lyz2} mice, compared to those receiving Eif2ak3^{Flox} controls (FIG. 3C). Also, significant reductions in the ability to blunt T cell proliferation and in the expression of PERK and Arginase I, but not in IRE1α or NOS2, were detected in tumor-MDSC from Eif2ak3^{KO-Lyz2} mice compared to Eif2ak3^{Flox} controls (FIG. 3D-E). Notably, higher expansion of cells resembling M-MDSC, but not PMN-MDSC, was observed within tumors of Eif2ak3^{KO-Lyz2} mice, compared to controls (FIG. 10E), which did not correlate with alterations in the apoptosis marker Annexin V, or the unmitigated ER stress driver of apoptosis, DR5 (FIG. 10F-G). To rule out the effect of the tumor burden differences in the actions induced by PERK deletion in MDSC, we obtained BM-MDSC from hematopoietic-endothelial specific Eif2ak3^{KO} (Eif2ak3^{KO-Tek}) mice, as PERK gene excision was ineffective in BM-MDSC from Eif2ak3^{KO-Lyz2} mice (FIG. 10H-K). Supporting the intrinsic role of PERK in UPR-primed MDSC, deletion of Eif2ak3 abolished the amplified T cell-inhibitory effect found in BM-MDSC treated with Thaps, without affecting the immunoinhibitory potential of vehicle-treated BM-MDSC (FIG. 3F). Furthermore, elimination of PERK did not impact the frequency of common myeloid progenitors (SMP) or granulocyte-macrophage progenitors (GMP) in the bone marrow of tumor bearing mice, nor affected the MDSC development from hematopoietic precursors (FIG. 10L-M), suggesting that the deletion of PERK did not alter the expansion of myeloid precursors or MDSC differentiation. Because of the major interaction between MDSC and T cell dysfunction, we then tested the role of T cells in the anti-tumor responses observed in Eif2ak3^{KO-Lyz2} mice. Augmented frequency of total, recently activated, and IFNγ-expressing CD8⁺ T cells was found in tumors from Eif2ak3^{KO-Lyz2} mice, compared to controls (FIG. 3G-I). Also, depletion of CD8⁺ T cells blunted the anti-tumor effects noticed in Eif2ak3^{KO-Lyz2} mice (FIG. 3J), suggesting the development of protective CD8⁺ T cell immunity in tumor-bearing PERK-null mice.

To recapitulate our findings in therapeutic models, we next evaluated the anti-tumor effects of two small-molecule PERK inhibitors, GSK-2606414, a dual PERK and RIPK1 inhibitor (Rojas-Rivera et al., 2017), and AMG-44, a potent and selective PERK inhibitor (Smith et al., 2015). Delayed tumor growth was noticed in B16-bearing mice treated with GSK-2606414, compared to vehicle-treated controls, which correlated with a lower immunoinhibitory activity of MDSC (FIG. 10N-O). A primary limitation for the therapeutic use of GSK-2606414 is the induction of hyperglycemia as a result of pancreatic islet degeneration (Yu et al., 2015). Therefore, we tested the therapeutic action of two different doses of AMG-44 (12 and 24 mg/kg). A similar delay in tumor growth, without alterations in the blood glucose levels or in the size or insulin expression in the pancreatic islets was found in B16-bearing mice treated with both AMG-44 doses (FIG. 3K, FIG. 10P-R). Moreover, illustrating the therapeutic potential of low-dose AMG-44, treatment of B16-bearing mice with AMG-44 impaired the immunoinhibitory activity of tumor-MDSC, induced expansion of tumor-infiltrating CD8⁺ T cells expressing IFNγ, and synergized with anti-PD-L1 immunotherapy (FIG. 3L-N). Notably, the therapeutic anti-tumor action of AMG-44 was detected in PERK-competent mice, but not in Eif2ak3^{KO-Lyz2} counterparts (FIG. 10S), suggesting the role of myeloid cell-PERK in the effects induced by AMG-44. Next, we tested the intrinsic effects of AMG-44 on Thaps-treated MDSC. Similar to our data in Eif2ak3^{KO-Tek} MDSC (FIG. 3F), AMG-44 overcame the ability of Thaps-treated BM-MDSC to impair T cell proliferation, without affecting MDSC-baseline activity (FIG. 3O). Thus, data demonstrate the crucial role of PERK in the regulatory function of MDSC, and the therapeutic potential of short-term treatments with low-dose PERK inhibitors as a strategy to overcome MDSC-linked T cell dysfunction and to enhance the efficacy of PD-L1 checkpoint blockade strategies.

(4) PERK Deletion Reprograms Tumor-MDSC into Immune-Stimulatory Cells.

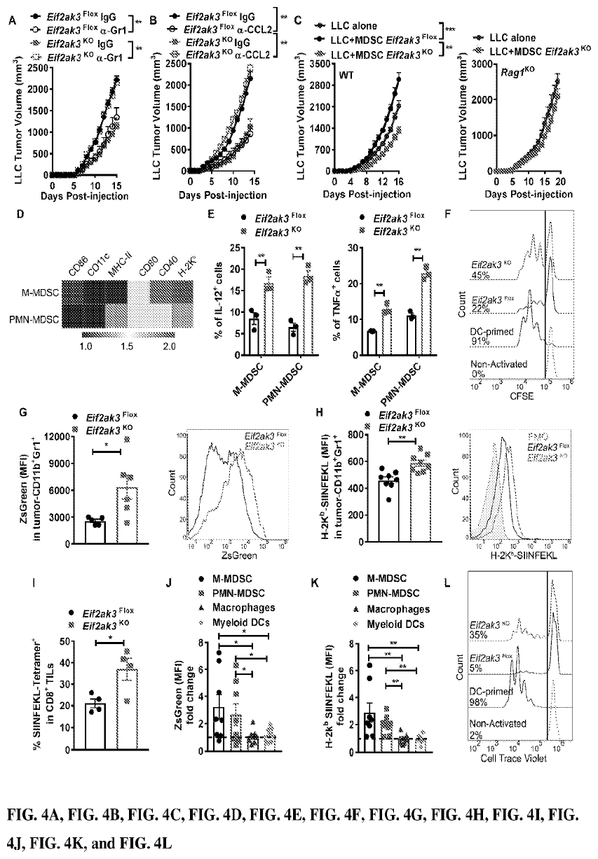

We evaluated whether the elimination of PERK functionally transformed MDSC into cells that promoted anti-tumor T cell responses. Deletion of MDSC-like cells after continuous injections with anti-Gr1 antibody, or inhibition of myeloid cell mobilization to tumors using anti-CCL2 antibody (Liang et al., 2017), restored tumor growth in LLC-bearing Eif2ak3^{KO-Lyz2} mice, while delaying tumor growth in Eif2ak3^{Flox} mice (FIG. 4A-B). Also, co-injection of tumor-infiltrating Eif2ak3^{KO-Lyz2} CD11b⁺ Gr1⁺ cells and LLC cells into C57BL6 mice (WT), but not into Rag1^{KO} mice, resulted in slower tumor growth, compared to LLC cells mixed with Eif2ak3^{flox} tumor-MDSC or tumor cells injected alone (FIG. 4C), suggesting a potential transformation of PERK-null MDSC into immune-stimulatory cells. Next, we compared by flow cytometry the expression of major co-stimulatory and pro-inflammatory myeloid markers in tumor M-MDSC (CD11b⁺ Ly6G^{high}Ly6C^{high}) and PMN-MDSC (CD11b⁺ Ly6G^{high} Ly6C^{low}) from LLC-bearing Eif2ak3^{KO-Lyz2} or Eif2ak3^{Flox} mice. Higher levels of MHC-I (H-2K^{b}), CD40, and CD80; but not MHC-II, CD11c, and CD86 were found in Eif2ak3^{KO-Lyz2} M-MDSC compared to controls, whereas no significant changes were found on PMN-MDSC (FIG. 4D). Moreover, increased expression of the inflammatory cytokines IL-12 and TNFα was detected in tumor-related M-MDSC and PMN-MDSC from Eif2ak3^{KO-Lyz2} mice compared to controls (FIG. 4E). Next, we studied whether the deletion Lyz2 of PERK allowed tumor-MDSC to process and present exogenous antigens. Eif2ak3^{KO-Lyz2} tumor-MDSC pulsed with complete OVA showed higher ability to prime naïve OT-I T cell proliferation, though lower than DCs, compared to control MDSC (FIG. 4F). Moreover, we tested the ability of Eif2ak3KO-Lyz2 MDSC to engulf tumor products and to cross-present antigens in the TME through s.c. injections with Pan02-OVA-ZsGreen tumors, which were engineered to express fluorescence protein ZsGreen and OVA. Elevated ZsGreen fluorescence and SIINFEKL-bound H-2K^{b} were detected on tumor-MDSC from Eif2ak3^{KO-Lyz2} mice compared to controls (FIG. 4G-H, FIG. 11A), and correlated with higher infiltration of tumor-specific SIINFEKL-H-2K^{b}-tetramer⁺ CD8⁺ T cells (FIG. 4I). Notably, deletion of PERK increased the expression of ZsGreen and SIINFEKL-bound H-2K^{b} in tumor-related M-MDSC and PMN-MDSC, but not in macrophages or myeloid DCs counterparts (FIG. 4J-K). Also, in agreement with the ability of PERK-deficient MDSC to present endogenously engulfed tumor antigens to CD8⁺ T cells, tumor-CD11b⁺ Gr1⁺ cells from Eif2ak3^{KO-Lyz2} mice bearing Pan02-OVA-ZsGreen tumors, but not from Eif2ak3^{flox} mice, induced ex vivo proliferation of naïve OT-I CD8⁺ T cells (FIG. 4L), but not of OT-II CD4⁺ T cells (FIG. 11B). Similarly, heightened expression of SIINFEKL-bound H-2K^{b} and higher capacity to prime OT-I T cell proliferation were found in tumor-MDSC from TUDCA-treated mice bearing Pan02-OVA-ZsGreen tumors (FIG. 11C-D). Altogether, results show that deletion of PERK or mitigation of UPR signaling transform MDSC into immune-stimulatory cells that activate CD8⁺ T cells within tumor beds.

(5) PERK Deletion Functionally Transforms MDSC Through Inhibition of NRF2

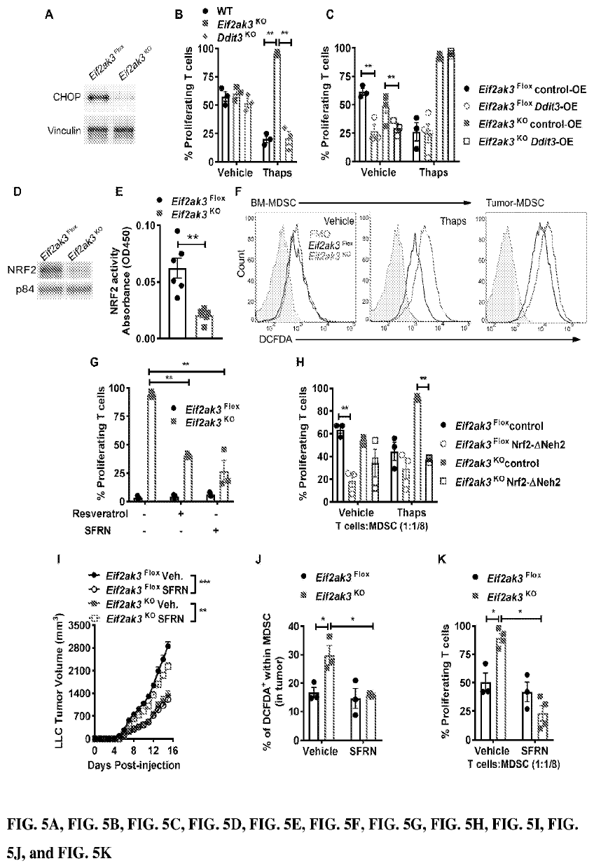

To identify the mediators by which PERK drives MDSC activity, we initially focused on CHOP (coded by Ddit3 gene), a downstream target of unmitigated cellular stress regulated by PERK, and previously linked to MDSC function (Thevenot et al., 2014). Lower levels of CHOP were detected in tumor-MDSC from Eif2ak3^{KO-Lyz2} mice compared to Eif2ak3^{Flox} controls (FIG. 5A). However, the dramatic reduction of MDSC-suppressive activity found in Eif2ak3^{KO-Tek} BM-MDSC treated with Thaps was not observed in Ddit3^{KO} BM-MDSC (FIG. 5B). Moreover, the ectopic expression of Ddit3 (FIG. 12A) failed to restore the immunosuppressive function of Eif2ak3^{KO-Tek} BM-MDSC after treatment with Thaps, while it enhanced the immunoinhibitory potential of vehicle-treated MDSC (FIG. 5C). These results ruled out the impact of an altered expression of CHOP in the impaired immunosuppressive function of PERK-null MDSC.

In light of the described phosphorylation of NRF2 by active PERK in tumor cells (Cullinan et al., 2003), we next studied the mechanistic interaction between PERK and NRF2 in MDSC. Similar to the PERK upregulation in tumor-MDSC (FIG. 1B), we noted higher levels of nuclear NRF2 in tumor-MDSC from LLC-bearing mice, compared to splenic MDSC and iMC counterparts (FIG. 12B). In addition, deletion of Nfe2l2 (NRF2-encoding gene) reduced the suppressive activity of tumor-MDSC to a similar extend as that induced by Eif2ak3 elimination (FIG. 12C). Consistent with the upstream role of PERK on NRF2 signaling, reduced expression of nuclear NRF2, lower binding of NRF2 to a consensus DNA sequence, and elevated ROS accumulation were found in tumor-MDSC and Thaps-treated BM-MDSC lacking PERK, compared to controls (FIG. 5D-F, FIG. 12D-E). Next, we confirmed the intrinsic relevance of a thwarted NRF2 signaling in the lower regulatory function of Thaps-treated Eif2ak3^{KO} MDSC. Treatment with the indirect NRF2-inducing agents, Resveratrol or Sulforaphane (SFRN), partially restored the potential of Thaps-treated BM-MDSC from Eif2ak3^{KO-Tek} mice to impair T cell proliferation (FIG. 5G). Consistently, enforced expression of NRF2-ΔNeh2, a dominant active form of NRF2, highly resistant to proteasome degradation (Shin et al., 2007) (FIG. 12F), restored the immunosuppressive activity and decreased ROS levels in Eif2ak3^{KO-Tek} BM-MDSC treated with Thaps, compared to the same cells transduced with a control vector, and to similar levels as those found in Thaps-treated flox MDSC carrying control or NRF2-ΔNeh2 vectors (FIG. 5H, FIG. 12G). Furthermore, treatment with SFRN disabled the anti-tumor effects found in Eif2ak3^{KO-Lyz2} mice, while delaying tumor progression in Eif2ak3^{Flox} mice (FIG. 5I). Also, treatment of LLC-bearing mice with SFRN reduced ROS levels and restored the immunoregulatory potential of tumor Eif2ak3^{KO-Lyz2} MDSC, without affecting controls (FIG. 5J-K). These results support the role of a reduced NRF2 signaling in the regulatory changes found in PERK-deficient MDSC.

(6) PERK regulates mitochondrial homeostasis in MDSC through NRF2

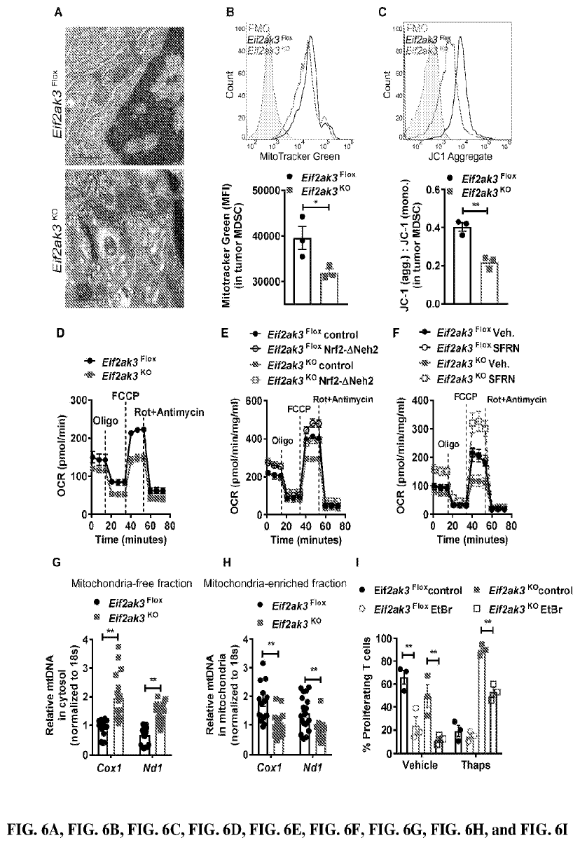

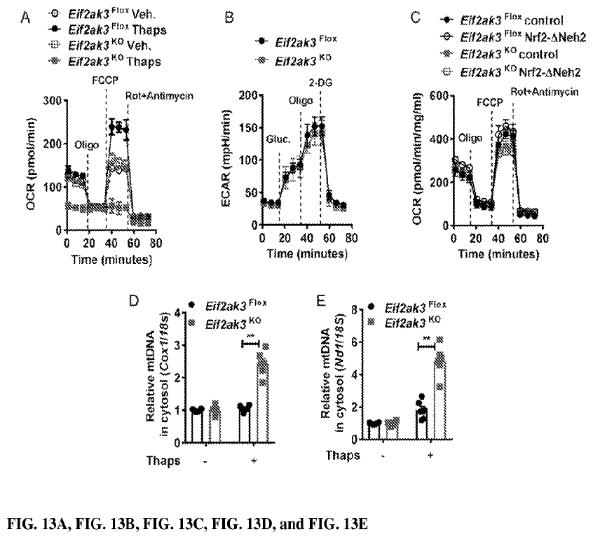

Decreased NRF2 activity can affect mitochondrial homeostasis (Cullinan et al., 2003). Thus, we determined whether the elimination of PERK impacted mitochondrial function in tumor-MDSC. Altered mitochondria morphology, diminished mitochondrial mass, and disrupted mitochondrial membrane potential were noted in Eif2ak3^{KO-Lyz2} MDSC from tumors compared to flox MDSC counterparts (FIG. 6A-C). Also, in comparison to wild-type MDSC, tumor-MDSC or Thaps-treated BM-MDSC from Eif2ak3-deficient mice showed a lower mitochondrial respiratory activity, as suggested by a reduced oxygen consumption rate (OCR) upon mitochondrial stress studies (FIG. 6D, FIG. 13A). Notably, no differences in the extracellular acidification rate (ECAR) were found between tumor-MDSC from control and Eif2ak3^{KO-Lyz2} mice after glycolysis stress profiles (FIG. 13B), indicating that PERK deletion promoted metabolic alterations linked to mitochondrial dysfunction rather than glycolysis. Next, we identified the role of a diminished NRF2 signaling in the mitochondrial alterations found in PERK-null MDSC. Enforced NRF2-ΔNeh2 expression restored mitochondrial respiratory activity, as tested by OCR, in Eif2ak3^{KO-Tek} BM-MDSC treated with Thaps (FIG. 6E), without inducing variations in control MDSC (FIG. 13C). In agreement, MDSC from tumor-bearing Eif2ak3^{KO-Lyz2} mice treated with SFRN had elevated OCR, compared to tumor-MDSC from untreated counterparts or from treated or untreated Eif2ak3^{Flox} mice (FIG. 6F). Thus, lower NRF2 signaling drives the mitochondrial respiratory dysfunction observed in PERK-null MDSC undergoing UPR activation.

Because of the emerging role of the cytosolic mtDNA as a mediator of innate immunity (Sliter et al., 2018; West et al., 2015) and the contribution of NRF2 signaling in maintaining mitochondrial integrity, we then assessed the presence of the mtDNA genes, Cox1 and Nd1, in mitochondria enriched and depleted cellular extracts from tumor-MDSC. Elevated mtDNA amounts in mitochondrial-free cytosolic extracts and reduced mtDNA content in mitochondrial enriched extracts were found in tumor-MDSC from Eif2ak3^{KO-Lyz2} mice, compared to MDSC counterparts from Eif2ak3^{Flox} controls (FIG. 6G-H). Also, Eif2ak3 deletion induced mtDNA accumulation in mitochondrial-free cytosolic extracts from BM-MDSC treated with Thaps, but not from non-treated BM-MDSC (FIG. 13D-E). Interestingly, depletion of cytosolic mtDNA with ethidium bromide (Wang et al., 2018) partially restored the suppressive activity of Eif2ak3^{KO-Tek} BM-MDSC treated with Thaps (FIG. 6I), further supporting the key role of the amplified cytosolic mtDNA content in the functional reprogramming of UPR-active PERK-null MDSC.

(7) PERK Ablation Provokes STING-Dependent Induction of Type I IFNs in MDSC

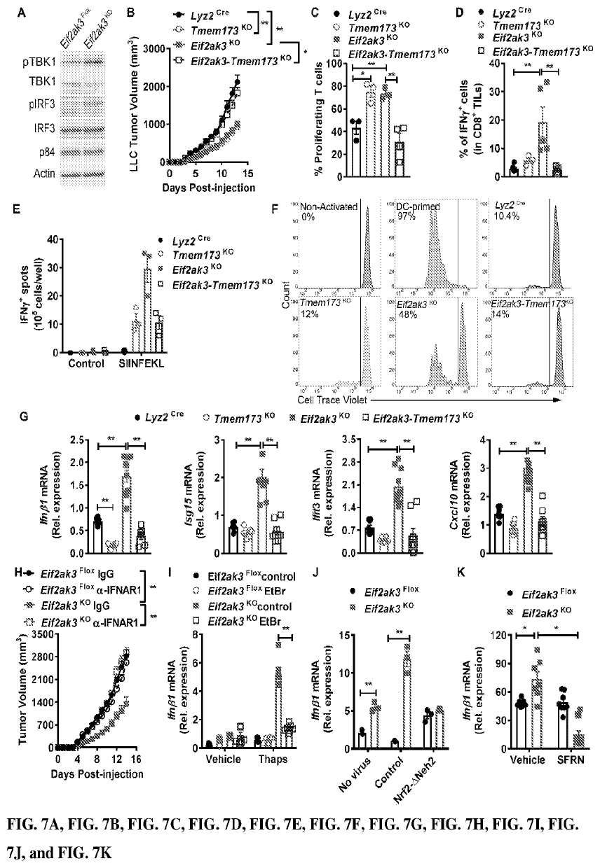

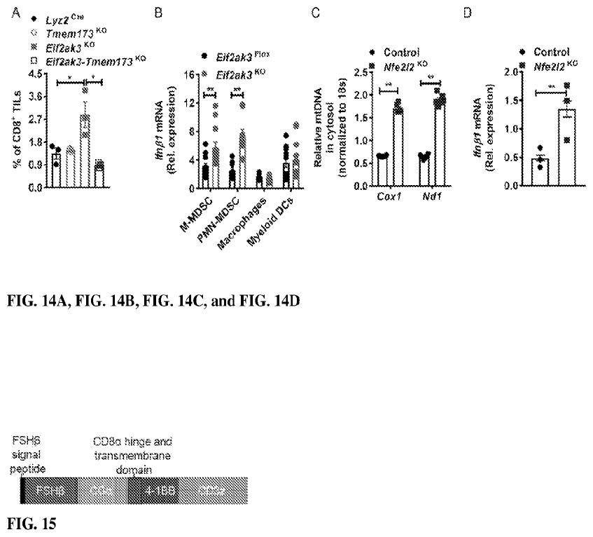

Presence of the mtDNA in the cytosol has been linked to stimulation of STING pathway (Sliter et al., 2018; West et al., 2015). Therefore, we compared the activation of the STING pathway in tumor-MDSC from Eif2ak3^{KO-Lyz2} mice and Eif2ak3^{Flox} controls. Heightened STING signaling, as shown by a higher phosphorylation of TBK1 and nuclear IRF3, was detected in tumor-MDSC from Eif2ak3^{KO-Lyz2} mice, compared to controls (FIG. 7A). Next, we sought to determine the role of STING in the anti-tumor effects induced by PERK deletion in MDSC, using myeloid cell-conditional PERK and STING-null mice (Eif2ak3-Tmem173^{KO-Lyz2}). Dual deletion of Eif2ak3 and Tmem173 in myeloid cells restored tumor growth and the ability of tumor-MDSC to blunt T cell proliferation, compared to Eif2ak3^{KO-Lyz2} or Tmem173^{KO-Lyz2} mice (FIG. 7B-C). In addition, Eif2ak3-Tmem173^{KO-Lyz2} mice showed lower infiltration of total and IFNγ-producing CD8⁺ T cells in tumors, compared to Eif2ak3^{KO-Lyz2} mice (FIG. 7D, FIG. 14A). Similarly, deletion of STING limited the development of an anti-OVA CD8⁺ T cell response in the draining lymph nodes, and ablated the ex vivo CD8⁺ T cell cross-priming activity found in tumor-MDSC from Eif2ak3^{KO-Lyz2} mice bearing Pan02-OVA-ZsGreen tumors (FIG. 7E-F). Also, consistent with STING activation, we observed augmented expression of the Type I IFNs-related transcripts, Ifnβ1, Isg15, Ifit3, and Cxcl10, in tumor-MDSC from Eif2ak3^{KO-Lyz2} mice and this was attenuated after Tmem173 deletion (FIG. 7G). Interestingly, the upregulation of Ifnβ1 mRNA noticed in tumor-MDSC subsets from Eif2ak3^{KO-Lyz2} mice was not found in macrophages and myeloid DCs, indicating a preferential effect on MDSC (FIG. 14B). Next, we tested the contribution of the Type I IFNs in the anti-tumor effects noted in Eif2ak3^{KO-Lyz2} mice. Antibody-based blockade of IFNα/β receptor subunit 1 (IFNAR1) restored tumor growth in Eif2ak3^{KO-Lyz2} mice compared to isotype IgG-treated controls (FIG. 7H). Thus, our results reveal a role for STING-dependent Type I IFNs in the anti-tumor effects induced by PERK deletion in myeloid cells.

To define the mediators by which PERK deletion triggered the production of Type I IFNs, we determine the effect of the accumulation of cytosolic mtDNA. Depletion of cytosolic mtDNA with ethidium bromide prevented the induction of Ifnβ1 mRNA in Eif2ak3^{KO-Tek} BM-MDSC treated with Thaps (FIG. 7I), supporting the role of the cytosolic mtDNA in the induction of Type I IFNs in PERK-null MDSC. Next, we evaluated the upstream role of a blunted NRF2 signaling in the upregulation of Type I IFNs in PERK-null MDSC. Similar to the effects found in Eif2ak3-ablated MDSC, heightened cytosolic mtDNA and Ifnβ1 mRNA levels were detected in tumor-MDSC from Nfe2l2-null mice, compared to those from controls (FIG. 14C-D). Also, ectopic expression of NRF2-ΔNeh2 prevented the induction of mRNA in Thaps-treated Eif2ak3^{KO-Tek} BM-MDSC, compared to controls (FIG. 7J). In agreement, tumor-MDSC from Eif2ak3^{KO-Lyz2} mice treated with SFRN showed reduced Ifnβ1 mRNA levels, compared to those from untreated PERK-null mice (FIG. 7K). Thus, results show the crosstalk between a reduced NRF2 signaling, cytosolic mtDNA, and STING-dependent production of Type I IFN as mediators for the immune-stimulatory effects induced by PERK deletion in MDSC.

b) Materials and Methods

(1) Mice

Experiments using mice were developed through an approved Institutional Animal Care and Use Committee (IACUC) protocol (IS00004043) and an active Institutional Biosafety Committee (IBC) study (#1385), both reviewed by the Integrity and Compliance board at the University of South Florida and Moffitt Cancer Center. Thus, the presented work has complied with all the relevant ethical regulations for animal testing and research. Wild type C57BL/6J mice (6 to 8 weeks) were from Envigo (Huntingdon, UK). Rag1^{KO} mice (NOD.129S7 (B6)-Rag1^{tmMom}/J), Lyz2^{cre} mice (B6.129P2-Lyz2^{tm1(cre)Ifo}/J), Tek^{cre} mice (B6.Cg-Tg(Tek-cre)1Ywa/J), Eif2ak3^{Flox} mice (Eif2ak3tm1.2Drec/J), Nfe2l2^{KO} mice (B6.129X1-Nfe2l2tm1Ywk/J), Td-Tomato reporter mice (B6.Cg-Gt(ROSA)26Sor^{tm9(CAG-tdTomato)Hze}/J), and OT-I mice (C57BL/6-Tg (Tcra-Tcrb) 1100Mjb/J) were from the Jackson laboratories (Bar Harbor, Me.). Tmem173^{Flox} mice were a kind gift from Dr. John C. Cambier (University of Colorado Denver and National Jewish Health). LSL-K-Ras^{G12D+} Trp53^{fl/fl} mice were developed in (Rutkowski et al., 2015). Eif2ak3^{KO-Lyz2} mice were created after breeding Eif2ak3^{Flox} mice with Lyz2^{cre} mice; while Eif2ak3^{KO-Tek} mice were obtained by crossing Eif2ak3^{Flox} and Tek^{cre} mice. Eif2ak3-Tmem173^{KO-Lyz2} mice were developed after breeding Eif2ak3^{KO-Lyz2} mice, Tmem173^{Flox} mice, and Lyz2^{cre} mice. Tmem173^{KO} mice were developed after breeding Tmem173^{Flox} and Lyz2^{Cre}. Lyz2-Td-Tomato and Tek-Td-Tomato reporter mice were developed after crossing Lyz2^{cre} and Tek^{cre} mice with Td-Tomato floxed mice. Mice of the same sex were randomly assigned to all experimental cohorts. All mice were maintained under specific pathogen-free conditions and used at 6-10 weeks of age.

(2) Human Materials

All human studies were covered through the approved Institutional Review Board (IRB) exempt protocol #19223, previously reviewed by the Regulatory Affairs Committee Board at Moffitt Cancer Center. Human peripheral blood mononuclear samples (PBMC) were obtained from One-Blood, Tampa, Fla. Human bone marrow stem cells were from a repository established by Dr. Pilon-Thomas (Moffitt Cancer Center). Both males and females were included in the donor pools and sample information maintained anonymous. All de-identified patients signed approved consent forms. Also, Met-NSCLC and high-grade serous ovarian cancer tissue microarrays (TMA) were available from the Moffitt Cancer Center Biorepository.

(3) Cell Lines

Lewis lung carcinoma (LLC; #CRL-1642), B16-F10 (#CRL-6475), and EG7 (#CRL-2113) were used for s.c. tumor models and obtained from the American Type Culture Collection (ATCC). Ovarian ID8-Defb29/Vegf-a and Pan02-Ova-ZsGreen cells lines were provided by Dr. Jose Conejo-Garcia and Dr. Shari Pilon Thomas, respectively (Perales-Puchalt et al., 2017; Svoronos et al., 2017). HEK293T cells (#CRL-11268) were obtained from ATCC. All cell lines were validated to be mycoplasma-free using the Universal Mycoplasma Detection Kit (#30-1012K, ATCC), and cultured in RPMI-1640 supplemented with 2 mM L-glutamine, 10 mM HEPES, 150 U/ml streptomycin, 200 U/ml Penicillin, 20 μM β-mercaptoethanol and 10% heat-inactivated Fetal bovine serum (FBS), and maintained at 37° C. in a humidified incubator with 5% CO₂.

(4) Tumor Models

Mice were subcutaneously (s.c.) injected with LLC, B16-F10, EG7 or Pan02-Ova-ZsGreen and tumor volume assessed using calipers and calculated using the formula [(small diameter)²×(large diameter)×0.5]. For the ovarian carcinoma model; ID8-Defb29/Vegf-a cells were injected intraperitoneal (i.p.) and body weight was assessed daily and mice euthanized when they gained 30% of their body weight. To develop soft tissue autochthonous flank sarcomas, mice with latent mutations in LSL-K-Ras^{G12D+} Trp53^{fl/fl} mice were irradiated for two consecutive days with 550 rads, followed by reconstitution with bone marrow from Eif2ak3^{Flox} or Eif2ak3^{KO-Lyz2} mice. Autochthonous flank sarcomas were then initiated six weeks later by intramuscular delivery of 2.5×10⁸ plaque-forming units of adenovirus coding for Cre recombinase (Gene Transfer Vector Core, University of Iowa) (Rutkowski et al., 2015). PERK inhibitors AMG-44 (12 or 24 mg/kg), GSK-2606414 (25 mg/kg) were administered i.p. daily and starting at day 6 post-tumor implantation and until study endpoint. Furthermore, mice received i.p. TUDCA (250 mg/kg) or Thapsigargin (Thaps, 100 μg/kg) after tumors were established (day 6 post-LLC injection). To deplete CD8⁺ T cells, tumor-bearing mice were injected i.p. with 400 μg α-CD8 antibody (clone 53-6.7, BioXcell) at day 0 followed by every 3^{rd} day treatments until experimental endpoint. Same approach was applied for elimination of Gr1⁺ cells using α-Gr1 antibody (250 μg/mouse, clone RB6-8C5, BioXcell), prevention of myeloid cells mobilization using α-CCL2 (250 μg/mouse, clone 2H5, BioXcell), blockade of PD-L1 (250 μg/mouse, clone 10F.9G2, BioXcell), and neutralization of interferon type 1 receptor using α-IFNAR1 (1 mg/mouse, clone MAR1-5A3, BioXcell). For NRF2 signaling induction, tumor-bearing mice were treated i.p. with D, L-Sulphoraphane (25 mg/kg, 3 days per week) starting at day 6 post-tumor injection.

Development of mouse and human MDSC from bone marrow (BM) precursors

Mouse BM-MDSC were generated after culturing BM precursors with GM-CSF and G-CSF (20 ng/ml each) for 4 days. In vitro generated MDSC were treated with either LLC tumor explant supernatants (TES, since day 0) or Thaps (200 nM, for the last 24 hours of culture). Human BM-MDSC were developed after culturing BM or peripheral blood precursors from healthy donors with GM-CSF and IL-6 (10 ng/ml each) for a period of 6 days (Thevenot et al., 2014). To induce UPR activation, MDSC were treated with either supernatants from renal cell carcinoma cells 786-0 (hTES) for 6 days (Rodriguez et al., 2009) or Thaps during the last 24 hours before collection (200 nM). In some experiments, MDSC were pre-incubated for 3 hours with Resveratrol (100 μM, Sigma-Aldrich), D,L-Sulphoraphane (20 μM), AMG-44 (5 μM), or TUDCA (500 nM) before treating with Thaps.

(5) Tumor Digestion

Minced tumors were digested with DNase I and Liberase (Roche USA, Branchburg, N.J.). Tumor digests were then treated with ACK buffer (Ammonium-Chloride-Potassium) to lyse the red blood cells.

(6) Cell Isolation

CD3⁺ T cells were enriched using mouse or human T cell negative selection kits (MagniSort, Invitrogen) from the spleen and lymph nodes of C57BL/6 mice; or from purchased human buffy-coat units (One-Blood, Tampa, Fla.). Purity ranged between 95% and 99% as tested by flow cytometry. For antigen-specific priming studies, CD8⁺ T cells were enriched using negative selection kits from the spleens of OT-I mice previously activated with OVA257-264 (SIINFEKL) for 48 hours. Tumor-MDSC were isolated from cellular suspensions of digested tumors followed by positive selection (MojoSort Streptavidin Nanobeads, Biolegend) using anti-Gr1 (Clone RB6-8C5, eBioscience) or anti-CD11b-biotinylated antibodies (Clone M1/70, StemCell Technologies, Vancouver). Also, M-MDSC (CD11b⁺Gr1⁺Ly6G^{low}Ly6C^{high}), PMN-MDSC (CD11b⁺Gr1⁺Ly6G⁺Ly6C^{low}), macrophages (CD11b⁺Gr1^{neg}F4/80⁺CD11c^{neg}), and myeloid DCs (CD11b⁺Gr1^{neg}F4/80^{neg}CD11c⁺) were sorted from tumors in a FACSAriaII (BD Biosciences).

(7) In Vitro and Ex Vivo Suppression Assays

Murine BM-MDSC (ratio 1:1/8) or tumor-MDSC (ratio 1:1/4) were co-cultured with negatively selected CD3⁺ T cells in a 96 well plate bound α-CD3 and α-CD28 (1 μg/ml each, eBioscience) for 3 days. T cell proliferation was assessed with Carboxyfluorescein succinimidyl ester (CFSE) or Cell-trace violet (Invitrogen) dye dilution detected by flow cytometry. For human studies, MDSC were co-cultured with enriched CD3⁺ T cells activated with soluble α-CD3 (1 μg/ml, clone OKT3) and α-CD28 (0.5 μg/ml, clone L293) for 3 days in a 96 well plate bound with goat anti-mouse IgG (10 μg/ml). Results are expressed as percentage of proliferating T cells, determined by the dilution of fluorescence compared with non-stimulated T cells.

(8) Antigen Presentation Co-Cultures

For antigen loading experiments, MDSC were sorted from LLC tumors and then pulsed with 1 μg/ml complete ovalbumin (OVA). After 16 hours, cells were washed twice and 2.5×10⁴ MDSC co-cultured with 1×10⁵ CFSE-labeled naïve CD8⁺ T cells from spleens of OT-I mice (ratio 0.25:1). Positive controls included co-cultures of OT-I T cells with GM-CSF plus IL-4-derived BM-DCs loaded with 1 μg/ml complete OVA. For TME cross-priming assays, 1.25×10⁴ MDSC from Pan02-Ova-ZsGreen tumors were co-cultured with 5×10⁴ naïve CD8⁺ T cells (1/4:1) from spleens of OT-I mice and labelled with Cell-trace violet. Positive control wells included OT-I T cells co-cultured with GM-CSF plus IL-4-derived BM-DCs loaded with 1 μg/ml OVA257-264 peptide. T cell proliferation was measured after 3 days of co-culture.

(9) Cloning of pHIV-OVA (deltaN)-2A-ZsGreen

The lentiviral constitutive expression vector pHIV-OVAdeltaN-2A-ZsGreen was created by cloning chicken ovalbumin lacking the first 49aa from the vector pcDNA3-deltaOVA (Addgene 64595) into the pHIV-T2A-ZsGreen vector backbone, which was generated from the pHIV-IRES-ZsGreen (Addgene 18121) by replacing the IRES sequence with the self-cleaving 2A sequence. Cloning reactions were performed according to manufacturer's recommendation using the InFusion-HD Cloning Plus kit from TakaraBio (#638909). Lentivirus containing supernatant was produced by transfecting pVSV-G, pSPAX2 and pHIV-OVA (deltaN)-2A-ZsGreen into LentiX-293T (TakaraBio). Collected supernatants were filtered (0.45 μm; ThermoFisher F2500-5) and lentivirus concentrated by ultracentrifugation at 24000 rpm for 2 hours at 4° C.

(10) Overexpression Vectors and Transduction of MDSC

Lenti-Ddit3 expressing vector was created by inserting the full-length Ddit3 cDNA into the lentiviral vector pCDH-MSCV-MCS-EF1α-GFP. Similarly, NRF2-ΔNeh2, a dominant active form of NRF2 highly resistant to proteasome degradation (Shin et al., 2007), was inserted in the same lentiviral vector. Empty vectors were used as negative controls. For virus production, 293T-cells (#CRL-3216, ATCC) were co-transfected with the lentiviral expression vector and a packaging vector that expresses the lentiviral envelope; and viral supernatants collected 48 and 96 hours post-infection. For lentiviral transduction, mouse BM precursors were incubated with GM-CSF and G-CSF (20 ng/ml each) and 24 hours later, MDSC transduced in the presence of 5 μg polybrene transfection reagent (Millipore) for 3 days until endpoint to perform readouts.

(11) Adoptive and Co-Transfer Experiments

For adoptive T cell therapy (ACT), mice bearing OVA⁺ EG7 tumors for 6 days received daily doses of TUDCA or vehicle until experimental endpoint. Additionally, specific cohorts of mice received on day 7 post-tumor injection 1×10⁶ CD8⁺ OT-I T cells sorted from OT-I splenocytes activated with 1 μg/ml OVA257-264 for 48 hours. In MDSC co-injection experiments, 1×10⁶ tumor MDSC from Eif2ak3^{Flox} and Eif2ak3^{KO} mice bearing LLC tumors were co-injected s.c. with 1×10⁶ LLC cells in 1:1 ratio into C57BL/6J mice or Rag1^{KO} mice.

(12) IFNγ ELISpot

Spleens from EG7-bearing mice undergoing or not ACT were collected 5 days after T cell transfer and 1×10⁵ splenocytes seeded in ELISpot plates containing pre-bound IFNγ capturing antibody in the presence or the absence of 1 μg/ml OVA257-264. Also, tumor draining lymph nodes from Pan02-Ova-ZsGreen-bearing mice were collected at endpoint and 1×10⁵ cells seeded in ELISpot plates containing pre-bound IFNγ capturing antibody in the presence or the absence of 1 μg/ml OVA257-264. Production of IFNγ was detected 24 hours later by ELISpot using Mouse IFNγ ELISpot (eBioscience).

(13) Flow Cytometry Staining

The conjugated antibodies and probes used for flow cytometry are listed in the Key Resources Table. For surface staining, cells were labelled with the appropriate antibodies in the presence of Fc blocker. For intracellular staining, surface-labeled cells were fixed with Cytofix/Cytoperm™ Solution (BD Biosciences), washed in Perm/Wash™ 1× solution, and labelled with intracellular antibodies. Cells were then washed in Perm/Wash™ 1× and PBS. Live vs. dead cell discrimination was performed prior to antibody labeling by Zombie Fixable Viability dye (Biolegend). Ex vivo intracellular staining for IL-12 and TNFα was performed on isolated cells after stimulation for 6 hours with LPS (1 μg/ml, Sigma Aldrich) in the presence of Golgi stop (0.8 μl/ml, BD Biosciences). For IFNγ staining, cells were incubated for 5 hours with phorbol myristate acetate (PMA, 750 ng/mL, Sigma Aldrich) and ionomycin (50 μg/mL, Sigma-Aldrich) in the presence and Golgi stop (0.8 μl/ml). For ER-tracker and Mitotracker staining, cells were probed with 100 nM of ER tracker green or 200 nM of Mitotracker green (Invitrogen) and then stained for surface markers. For Mitochondrial membrane potential, cells were stained with JC-1 flow cytometry assay kit (Cayman chemicals) followed by surface markers staining. ROS were detected by DCFDA (10 μM) or Dihydroethidium (DHE, 10 μM). Data acquisition was performed in a CytoFLEX II (Beckman Coulter) or LSRII (BD Biosciences). All analysis was performed using FlowJo version 11 software.

(14) Immunoblot Analysis

Equal protein amounts of total and nuclear cell lysates were electrophoresed in 8 or 10% Tris-Glycine gels (Novex-Invitrogen), transferred to PVDF membranes by iBlot™ Gel Transfer Device (ThermoFisher), and blotted with the corresponding primary and secondary antibodies described in Key Resources Table. For nuclear fraction isolation, cells were lysed using NE-PER Nuclear and Cytoplasmic extraction kit (ThermoFisher). Membrane-bound immune complexes were detected by ChemiDoc™ Imaging System (Bio-Rad, #17001401).

(15) Quantitative PCR

Total RNA was isolated from MDSC using TRIzol (Life Technologies). Reverse transcription was performed using Verso cDNA Synthesis Kit (Thermo Scientific). Quantitative PCR reactions were prepared by using Bio-Rad SYBR green master mix and performed on an Applied Biosystems thermocycler (7900 HT) using primers described in Key Resources Table

(16) NRF2 Binding Activity

Nucleic protein extracts (15 μg) from tumor-MDSC or in vitro-generated MDSC were monitored for binding of NRF2 to a DNA consensus sequence though a TransAM® Kit (Active Motif).

(17) Seahorse Metabolism Assay

Oxygen consumption rate (OCR) through mitochondrial stress test and extracellular acidification rate (ECAR) by glycolysis stress profile were measured using a XF96 extracellular flux analyzer (Seahorse Bioscience). MDSC were plated onto CellTak pre-coated wells (1×10⁵ cells/well in triplicate), and subjected to mitochondrial stress and glycolysis stress protocols using specific non-buffered XF base mediums. For OCR, cells were analyzed under basal conditions and in response to 2 μM oligomycin, 2 μM fluorocarbonyl-cyanide-phenylhydrazone (FCCP), and 0.5 μM rotenone. For ECAR, cells were plated in XF media lacking glucose and monitored under basal conditions and in response to 10 mM glucose, 10 μM oligomycin, and 100 mM 2-Deoxy-D-glucose (2-DG). Final values were obtained after protein normalization among the conditions.

(18) Cellular Fractionation and Quantification of mtDNA

Cytosolic and mitochondrial cellular fractionation of BM-MDSC or tumor-MDSC (equal number of cells per each sample) was performed using mitochondrial isolation kit for mammalian cells (ThermoFisher) according to the manufacturer's protocol. MtDNA from the cytosolic and mitochondrial fractions was isolated and purified using DNeasy blood and tissue kit (Qiagen). MtDNA was quantified by qPCR using primers listed in Key Resources Table and normalized to a nuclear DNA sequence (18s gene).

(19) Mitochondrial DNA Depletion

BM-MDSC were developed in the presence or absence of 150 ng/ml of ethidium bromide (Sigma Aldrich) for 4 days. On day 4, cells were collected and in vitro suppression assays were conducted as described above.

(20) Immunofluorescence

Formalin fixed paraffin embedded TMA sections were stained using an automated OPAL-IHC system (PerkinElmer) in a BOND RX (Leica Biosystems). Briefly, slides were treated with the PerkinElmer blocking buffer for 10 min and incubated with the specific primary antibodies, followed by OPAL-HRP polymer and one OPAL fluorophore. Individual antibody complexes were stripped after each round of detection and DAPI applied as the last staining. Auto-fluorescence slides (negative control) included primary and secondary antibodies, omitting the OPAL fluorophores. Slides were imaged with a Vectra®3 Automated Quantitative Pathology Imaging System. Multi-layer TIFF images were exported from InForm (PerkinElmer) into HALO (Indica Labs) for quantitative image analysis. Each fluorophore was assigned to a dye color and positivity thresholds determined visually per marker based on nuclear or cytoplasmic staining patterns, and by intensity thresholds normalized for exposure (counts/2 bit depth×exposure time×gain×binning area). Cell segmentation results from each core were analyzed using FCS Express 6 Image Cytometry (De Novo software).

Hematoxylin and Eosin (H&E) stained slides were scanned in an Aperio AT2 whole slide scanner (Leica Biosystems Inc.) equipped with a 20×0.7NA objective lens Images were created at 0.5 micron per pixel resolution and imported into Definiens Tissue Studio software v4.7 (Definiens AG) for analysis. Islets were found using a semi-automated segmentation process. First an automatic segmentation was applied to the image to create contour lines around objects within the image. Objects that contained islets were classified as such and remaining objects were classified as non-islet tissue. Next, adjacent objects of the same classification were merged together to clean up the segmentation. Segmented images were analyzed to provide the total area of islets Immunofluorescence for mouse insulin in pancreas tissue sections was performed as we described (Sultan et al., 2017). Labeled samples were scanned with a Zeiss Imager Z2 Upright FL microscope with a 10× objective lens using the tile scan function. Images were created at a

0.65 micron per pixel resolution and imported into the Definiens Tissue Studio software v4.7 (Definiens AG) for islet detection and analysis. Islets were detected using a semi-automated segmentation process. First, an automatic segmentation was applied to the image to detect the tissue. Then, the software was trained to detect islet vs. non-islet tissue within the image. In order to remove false detected islets caused by background staining, detected islets larger than 150,000 square microns and less than 600 square microns were reclassified to non-islet tissue. Segmented images were then analyzed to provide the fluorescent intensity of islets and total tissue area.

(21) Electron Microscopy

Tumor-MDSC were fixed in 4% paraformaldehyde, 2% glutaraldehyde in 0.1M sodium cacodylate (NaCac) buffer, pH 7.4, post-fixed in 2% osmium tetroxide in NaCac, stained in block with 2% uranyl acetate, dehydrated with a graded ethanol series, and embedded in Epon-Araldite resin. Thin sections were cut with a diamond knife on a Leica EM UC6 ultramicrotome (Leica Microsystems, Inc, Bannockburn, Ill.), collected on copper grids and stained with uranyl acetate and lead citrate. Tissue was observed in a JEM 1230 transmission electron microscope (JEOL USA Inc., Peabody, Mass.) at 110 kV and imaged with an UltraScan 4000 CCD camera & First Light Digital Camera Controller (Gatan Inc., Pleasanton, Calif.). Quantitative analysis was performed with ImageJ (NIH, LOCI, University of Wisconsin-Madison)

(22) Quantification and Statistical Analysis

Statistical analysis was performed using GraphPad Prism 7 (San Diego, Calif.). For two groups, means were compared by two-tailed unpaired Student's t-test. For multiple groups and tumor growth studies, analysis was done by ANOVA with Bonferroni correction for multiple comparisons. P value of <0.05 was considered statistically significant. Specific statistical test results are indicated in each figure: *, p<0.05; **, p<0.01***, p<0.001.

c) Discussion

Here, we elucidated the profound impact of the UPR-related kinase PERK in the immune-suppressive polarization of MDSC in tumors. PERK deletion in MDSC reduced NRF2 signaling, which disrupted mitochondrial homeostasis and promoted STING-driven production of Type I IFNs. Our data suggest a new strategy to reprogram immunosuppressive myelopoiesis in tumors.

Although a moderate and transient activation of the UPR-associated signals supports the physiological differentiation and function of immune cells (Bettigole et al., 2015; Dong et al., 2019; Iwakoshi et al., 2007; Maranon et al., 2010; Reimold et al., 2001; Thaxton et al., 2017), it has also become evident that sustained and maladaptive stimulation of the UPR-drivers promotes immune dysfunction in tumors (Condamine et al., 2014; Cubillos-Ruiz et al., 2015; Thevenot et al., 2014). In fact, conditional deletion of IRE1α-XBP1 restored the ability of DCs from ovarian tumors to prime protective anti-tumor T cell immunity (Cubillos-Ruiz et al., 2015), and blocked the capacity of macrophages to promote cancer cell metastasis (Yan et al., 2016). Moreover, the expression of IRE1α-XBP1-driven lectin-type oxidized LDL receptor 1 enabled to differentiate PMN-MDSC from non-immunoregulatory neutrophils (Condamine et al., 2016). In addition to the actions of IRE1α-XBP1, upregulation of CHOP controlled the immunosuppressive activity and turnover of tumor-MDSC (Thevenot et al., 2014). Despite the aforementioned seminal reports that illustrated the significance of the UPR mediators in tumor-associated myeloid cells, the role and downstream signaling through which PERK regulates the immunosuppressive myeloid cells within tumors remained unknown. Our results revealed a major role of PERK in the immunoregulatory activity of tumor-MDSC. The critical function role of PERK in MDSC was only manifested after the exposure to the TME or activation of the UPR. In cancer cells undergoing unmitigated ER stress, the activation of PERK and IRE1α induced opposite and coordinated signals that controlled apoptotic vs. survival cell fate, respectively (Chang et al., 2018; Lu et al., 2014). Conversely, PERK deletion in tumor-MDSC did not correlate with intrinsic alterations in phospho-IRE1α levels or apoptosis rates, suggesting that elimination of PERK in tumor-MDSC does not necessarily lead to compensatory IRE1α signaling or cell death. The physiological activities of PERK have been mostly attributed to the phosphorylation of eIF2α (Cullinan and Diehl, 2004; Cullinan et al., 2003) and induction of CHOP (Harding et al., 2000). However, the immunostimulatory actions induced upon the ablation of PERK in tumor-MDSC occurred in a CHOP-independent manner Instead, PERK deletion transformed tumor-MDSC through NRF2 signaling inhibition, which supports previous reports in cancer cells showing the crosstalk between PERK and NRF2 in the survival to high levels of ROS (Cullinan and Diehl, 2004). Rather than regulation of survival, our data showed that the inhibition of NRF2 signaling in PERK-deficient MDSC triggered ROS accumulation and thwarted mitochondrial respiratory function. The paradoxical increase in ROS was intriguing, as previous reports showed the key role of the production of ROS by Nox2 in the regulatory activity of MDSC (Corzo et al., 2009; Raber et al., 2014). Consistent with the immunostimulatory role of amplified ROS in MDSC, a recent study demonstrated the relevance of the ROS elevation in the p53-dependent reprogramming of M-MDSC into CD103⁺ DCs (Sharma et al., 2018). These results suggest the multi-faced and paradoxical role of the regulation of ROS in the functionality of MDSC.

Our findings uncovered the role of STING in the reprogramming effects triggered by PERK deletion in tumor-MDSC. In agreement, STING-mediated Type I IFNs in DCs effectively incited anti-tumor immunity (Corrales et al., 2015; Deng et al., 2014; Woo et al., 2014). Our results show that reduced NRF2 activity upon PERK deletion reprogrammed tumor-MDSC into immunocompetent myeloid cells through dysregulation of mitochondrial respiratory activity, accumulation of cytosolic mtDNA, and subsequent activation of STING-driven production of Type I IFNs. These data support the emerging role of cytosolic mtDNA as an intrinsic mediator of STING activation and innate immunity (Sliter et al., 2018; West et al., 2015). Also, deletion of PERK promoted the expression of Type I IFNs in tumor-MDSC subsets, but not in macrophages and myeloid DCs counterparts, which could be related with the active degradation of cytosolic mtDNA in macrophages (Xu et al., 2017) and the incomplete gene excision in myeloid DCs using the Lyz2-Cre recombinase system. Although we showed the indirect induction of STING-driven Type I IFNs by reduced NRF2 through expansion of cytosolic mtDNA, a recent report demonstrated an alternative negative effect of NRF2 on STING mRNA stability (Olagnier et al., 2018). Notably, although STING regulated the immunostimulatory transformation of PERK-null MDSC, we also observed that myeloid cell-STING promoted tumor growth in PERK-competent mice. The pro-tumor vs. anti-tumor roles of STING can be argued in the light of seminal reports that illustrated the paradoxical effects of STING in tumors. STING activation was shown to prime effective anti-tumor immunity against immunogenic and spontaneously rejected tumors (Woo et al., 2014), whereas it promoted tumor growth in non-immunogenic tumor models (Lemos et al., 2016). The magnitude of STING signaling in response to the stimuli could be another explanation. Indeed, STING promoted inflammation driven carcinogenesis and tolerance towards spontaneously dying cells (Ahn et al., 2017; Ahn et al., 2014; Lemos et al., 2016), while it provoked anti-tumor immune responses following amplified cell death induced by irradiation or ROS triggered cytosolic DNA sensing (Carroll et al., 2016; Deng et al., 2014). An additional explanation for the pro-tumor effects of STING in PERK-competent mice include the occurrence of STING-to-Type I IFNs uncoupled events. In fact, PERK can directly limit spontaneous Type I IFN responses by induction of phosphorylation-mediated degradation of IFNAR1 (Bhattacharya et al., 2013; Yu et al., 2015). Also, STING regulates the activity of NFkB, a key driver of tumor-MDSC activity (Sierra et al., 2017). Thus, development of mechanistic studies elucidating the role of STING-Type I IFNs dependent vs. independent actions in tumor-myeloid cells could enable to clarify the paradoxical effects of STING and to design strategies to therapeutically reinvigorate anti-tumor immunity.

The immunoregulatory role of the relentless priming of UPR is not restricted to myeloid cells. TME-induced stimulation of IRE1α-XBP1 and PERK-CHOP signaling promote dysfunction in intratumoral T cells (Cao et al., 2019; Hurst et al., 2019; Song et al., 2018). Moreover, activation of the UPR regulates adaptation processes in cancer cells and controls their metastatic potential through promotion of survival, angiogenesis, or chemo-resistance, and by impairing anti-tumor immune surveillance (Bi et al., 2005; Chen et al., 2014; Lee et al., 2014; Wu et al., 2015). Interestingly, elimination of PERK in cancer cells decreased their adaptation to hypoxia, DNA damage, nutrient starvation, and high levels of ROS, resulting in delayed tumor growth (Bobrovnikova-Marjon et al., 2010; Maas and Diehl, 2015; Wu et al., 2015). As such, inhibition of PERK has emerged as a promising approach to block tumor growth and metastasis (Atkins et al., 2013; Maas and Diehl, 2015). However, the interest towards the use of PERK inhibitors in cancer has been affected by the reported pancreatic toxicity, which occurred in a Type I IFN-dependent manner (Yu et al., 2015). Our results indicate that short-term treatment with low dose PERK inhibitors promoted anti-tumor protective immunity and boosted the effectiveness of anti-PD-L1, without altering blood glucose levels or the size or insulin expression of the pancreatic islets. Despite these promising results, further pharmacological investigations evaluating the activity of PERK inhibitors is warranted, as we anticipate that the dose, treatment period, route, and bioavailability could regulate their therapeutic vs. toxic effects. Alternatively, therapeutic use of cellular stress mitigating agents, such as TUDCA, could target all branches of UPR, without known toxicity. Thus, it is likely that the inhibition of PERK, or mitigation of maladaptive UPR activation, could induce dual anti-tumor and immune-stimulatory effects in tumor-bearing hosts. This remains to be tested in clinical studies and could have a major impact in the improvement of cancer immunotherapy.

In summary, we demonstrate the primary role of PERK in the MDSC-induced CD8⁺ T cell dysfunction in tumors, and suggest the therapeutic inhibition of PERK as a promising intervention to restore protective anti-tumor myelopoiesis in individuals with cancer, which is expected to augment the promising efficacy of different forms of cancer immunotherapy.

### 2. Example 2: Targeting Immunosuppressive ER Stress to Render Cellular Immunotherapies Effective Against Cancer

Collaborative studies by the PIs have demonstrated that two major ER stress response pathways, IRE1α-XBP1 and PERK-Chop, cripple anti-cancer immunity by altering the function of myeloid cells and T cells in tumors. We showed that in the setting of immunotherapy-refractory ovarian cancer, maladaptive activation of IRE1α-XBP1 in intra-tumor T cells limits their anti-cancer activity by impairing mitochondrial respiration and metabolic fitness. In addition, we found that the sustained activation of PERK-Chop intrinsically thwarts the effector function of ovarian tumor-infiltrating T cells, which also correlated with an impaired mitochondrial metabolic activity and limited expansion of protective tissue-resident memory (T_{RM}) CD8+ T cells. We propose that the development of successful T cell-based therapies for solid tumors, like ovarian carcinoma, will require overcoming the detrimental ER stress-driven immunoregulatory signals, which remain largely unexplored.

This application represents a concerted effort by a team of long-term collaborators with high expertise in the immune defects induced by persistent ER stress, and in the development of T cell-based therapies in ovarian carcinoma. Our team has developed Chimeric Endocrine Receptors (CERs) that use the two subunits of the follicle-stimulating hormone (FSH) to target FSH-receptor positive (FSHR+) ovarian tumors. We have initiated the process of FDA approval for a clinical trial at Moffitt using transferred CER-transduced T cells against chemo-resistant ovarian cancer. We postulate that understanding the signals by which ER stress responses render CER-T cells dysfunctional during manufacturing and/or upon homing to the TME will pave the way for developing the next generation of effective cellular therapies against solid tumors, while leading to the identification of novel biomarkers to discriminate responding from non-responding patient cohorts. Our central hypothesis is that the persistent activation of ER stress sensors in FSH-CER-T cells during the expansion process and/or upon adoptive transfer into ovarian carcinoma-bearing hosts limits their anti-tumor activity by inhibiting their metabolic fitness and by preventing their differentiation into protective T_{RM} CD8+ T cells. Also, we propose that the specific abrogation of IRE1α and/or PERK signaling in adoptively-transferred CER-T cells, using novel pharmacologic and genetic approaches, will overcome the inhibitory effect of the TME and enable the implementation of cellular therapies that effectively eliminate solid malignancies. This hypothesis is based on major novel findings: First, the IRE1α-XBP1 axis blunts the effector activity and metabolic fitness of tumor-reactive T cells. Second, both Xbp1^{-/-} and Chop^{-/-} anti-cancer T cells exert superior control of tumor growth in mice, compared with control counterparts. Third, T_{RM} CD8+ T cells in human ovarian tumors show decreased expression of ER stress response gene markers. Fourth, ablation of Chop in tumor-reactive T cells enhances their capacity to differentiate into T_{RM} T cells in vivo in the TME.

a) Sustained ER Stress Responses Cripple Effective Anti-Cancer Immunity.

The impaired anti-tumor T cell immunity observed in most patients with ovarian carcinoma represents a major impediment to the success of different forms of immunotherapy. Several mechanisms triggered by the ovarian tumor microenvironment (TME) have been shown to block anti-cancer T cell responses, including the induction of hypoxia, the elevated production of reactive oxygen and nitrogen species, and the starvation of key nutrients, among others. These adverse conditions disturb the protein-folding capacity of the endoplasmic reticulum (ER) and induce activation of the unfolded protein responses (UPR), which is triggered as a cellular adaptation process. Our seminal studies uncovered that maladaptive ER stress responses cripple immunity to ovarian carcinoma. Three ER stress sensors initiate the UPR: the activating transcription factor 6 (ATF6), the inositol-requiring enzyme 1 (IRE1α), and the PKR-like ER kinase (PERK). Activation of IRE1α and PERK leads to the alternative splicing and activation of X-box binding protein 1 (Xbp1) mRNA and the induction of C/EBP-homologous protein (Chop), respectively, which operate as multitasking transcription factors with canonical functions in the UPR, but also with primary cell- and context-dependent activities capable of altering metabolic processes. We and others reported mutually-reinforcing evidence showing that the persistent activation of IRE1α-XBP1 or upregulation of Chop directly regulates the activity of various myeloid cell subsets in tumors. We further demonstrated that sustained IRE1α-XBP1 signaling intrinsically inhibits the anti-cancer capacity of intratumoral T cells by dampening their metabolic fitness. In addition, recent reports from us and others highlighted the regulatory role of PERK-Chop in anti-tumor T cell responses by limiting mitochondrial activity. In Aim 1 of this proposal, we postulate that disabling IRE1α-XBP1 and/or PERK-Chop in transferred anti-tumor T cells or pharmacological conditioning of the T cell-transfer recipients, will significantly improve their protective activity against ovarian cancer. This will be tested through our clinically-relevant models of ovarian carcinoma, mice conditionally lacking IRE1α-XBP1 and/or PERK-Chop in T cells, and immunotherapies based on recently developed Chimeric-Endocrine Receptors (CER) T cells that use the two subunits of the follicle-stimulating hormone (FSH) to target ovarian tumors expressing the FSH-receptor (FSHR+). Our research will have rapid clinical applicability as we have requested FDA approval for a clinical trial at Moffitt to test the effect of FSH-CER T cells in chemo-resistant ovarian cancer patients.

b) Aberrant IRE1α-XBP1 Signaling is a Metabolic Checkpoint in T Cells.

Metabolic competition and glucose restriction in the hostile microenvironment of solid tumors cause major glycolytic and bio-energetic defects in infiltrating T cells. In our recent collaborative study, we envisioned that these unfavorable conditions could disrupt ER homeostasis in intratumoral T cells. We found that glucose deprivation blocks N-linked protein glycosylation, thereby inducing ER stress and IRE1α-XBP1 activation in ovarian cancer-associated T cells. Under nutrient-rich conditions, IRE1α-XBP1 signaling induces the expression of genes that promote N-linked glycosylation and protein folding using glucose as a substrate. However, stimulation of IRE1α-XBP1 under glucose-limiting conditions evoked aberrant responses as this key nutrient was unavailable for restoration of ER homeostasis. We showed for the first time that IRE1α-XBP1 signaling in the absence of glucose blunted T cell mitochondrial respiration by controlling the abundance of glutamine carriers and limiting glutamine influx. Restoring N-linked protein glycosylation, abrogating IRE1α-XBP1 activation, enforcing glutamine carrier expression, or supplementing glutamine-derived tricarboxylic acid (TCA) cycle metabolites restored mitochondrial respiration and effector function in glucose-starved T cells undergoing ER stress. In sub-Aim 2A, we will define whether the ablation of IRE1α-XBP1 increases the ability of CER-T cells to eliminate FSHR+ ovarian tumors by augmenting glutamine-related mitochondrial fitness.

c) PERK-Chop Activation Restricts Anti-Cancer T Cell Function and Limits the Development of T_{RM} T Cells in Tumors.

ER stress-driven activation of PERK leads to phosphorylation of the eukaryotic translation initiation factor 2 alpha (eIF2α) and a subsequent induction of the activating transcription factor 4 (Atf4) and Chop. While the phosphorylation of eIF2α by PERK controls cancer cell survival or apoptosis through the induction of ATF4 and Chop, respectively, the direct effects of the PERK-Chop pathway in the dysfunction of tumor-associated T cells remain largely unexplored. Recent publications from us and others showed elevated mitochondrial activity and redox potential in T cells lacking PERK. Additionally, our preliminary unpublished studies highlight the potential relevance of this UPR arm in the development of tissue-resident memory (T_{RM}) CD8+ T cells in ovarian tumors, a T cell subset associated with protective anti-tumor immunity. T_{RM} CD8+ T cells control the magnitude of cytotoxic T-cell responses in tumors; respond much faster to re-exposure to cognate antigens; and show increased cytotoxic potential and mitochondrial lipid-metabolism. Although T_{RM} T cells are essential drivers of T cell-based immunotherapies, the primary signals regulating the expansion of T_{RM} T cells in tumors remain obscure. In sub-Aim 2B, we postulate that PERK-Chop intrinsically limits the differentiation of transferred FSH-CER T cells into T_{RM} T cells in tumors through transcriptional inhibition of key mediators of lipid metabolism that promote T_{RM} T cell differentiation.

d) ER Stress Activates NF-κB, Driving Inflammatory Responses.

NF-κB is a key transcription factor that orchestrates the expression of genes associated with inflammation, cell proliferation and survival. During the UPR, NF-κB is activated through multiple mechanisms. For instance, the kinase activity of activated IRE1α maintains IKK basal activity, which drives proteasomal degradation of IκBα and, subsequently, robust NF-κB activation. Priming of the PERK pathway results in phosphorylation of eIF2α, which inhibits the translation of IκBα, thereby activating NF-κB. Finally, PERK increases the DNA-binding capacity of NF-κB through interactions with STAT3. However, these mechanisms have been characterized in epithelial cells, and therefore their applicability in T lymphocytes remains to be investigated. This is important because NF-κB has been proposed as a common signal transducer driving rapid effector differentiation in memory T cells. Notably, CAR T cells from non-responding patients are characterized by the upregulation of programs involved in effector differentiation, while the infusion products of responders are enriched in memory-related genes. In Aim 3, we will define how modulation of ER stress responses influences NF-κB activity in CER-T cells, paving the way for the generation of infusion products with optimal in vivo efficacy and persistence.

Our proposal is significant because: 1) It will define whether the activation of ER stress responses during FSH-CER T cell manufacturing or after homing to the tumor milieu limit their anti-cancer capacity in ovarian carcinoma. This is expected to have a major impact in the field of T cell-based therapies by elucidating primary mechanisms regulating CAR/CER T cell effectiveness in solid tumors. 2) Our experiments will provide the foundation to develop CER/CAR T cells refractory to the harsh conditions in the TME by promoting T_{RM} T cell expansion and enhancing mitochondrial metabolic fitness. This is highly significant as T cell- and CAR-T-based immunotherapies remain unsuccessful in solid tumors. 3) The robust proposed approaches will use transplantable and autochthonous mouse ovarian tumor models, which will be validated with highly translational studies in human T cells and treatment models using human T cells transduced with FSH-CER-coding viruses. 4) Moffitt has a state-of-the-art FDA approved and CLIA-certified GMP Cell Therapy Facility (only 5 in USA). The experimental endpoints from the application will be integrated in the future with a clinical assay using FSH-CER in chemo-resistant patients with ovarian carcinoma (FDA IND approval pending).

These studies are highly innovative at multiple levels, including: 1) it is shown whether severe activation of ER stress sensors in the TME hinders the anti-ovarian cancer activity of transferred CER T cells. 2) It is shown that genetic ablation of IRE1α-XBP1 and/or PERK-Chop in recently developed anti-ovarian cancer FSH-CER T cells enhances their metabolic fitness and tumoricidal capacity upon arrival in the TME. 3) We will define the hitherto uninvestigated role of PERK-Chop in preventing the differentiation of T_{RM} T cells. 4) The distinct role of the IRE1α-XBP1 and PERK-Chop pathways in the differentiation of terminal effector vs. memory T cells during the generation of CER T cell infusion products for clinical use is dissected. 5) For the first time non-viral, CRISPR-based methods for the integration of our FSH-CER constructs while ablating the most characterized pathway of ER stress in human T cells.

e) Results

(1) Human and Mouse FSHR-Targeting CERs.

We generated new human and mouse 4-1BB-based chimeric receptors against FSHR+ ovarian cancer cells that include all the signals successfully used in leukemia patients. To target FSHR, we synthetized a construct expressing a signal peptide, followed by the two subunits of FSH (FSHβ and CGα, the latter common to LH and TSH), separated by a linker (FIG. 15). This targeting motif was cloned in frame with a hinge domain from CD8α, followed by the transmembrane domain of CD8α, the intracellular domain of co-stimulatory 4-1BB, and the activating CD3ζ domain. Because engagement of FSH with its endogenous FSH receptor is expected to occur in the absence of any antigen-antibody reaction, we termed this construct Chimeric Endocrine Receptor (CER). In parallel, we have generated CERs with the corresponding mouse sequences for expression in mouse T cells and detection of murine FSHR+ tumors.

(2) Human FSH-CER T Cells Kill Ovarian Cancer Cells in a Dose-Dependent Manner.

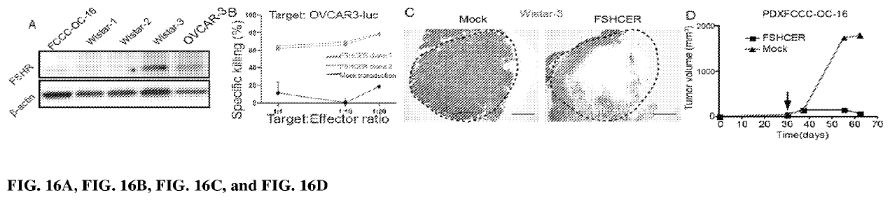

Human T cells transduced with human FSH-CER were tested for their anti-tumor activity against human ovarian cancer cells expressing different levels of FSHR (FIG. 16A). FSH-CER T cells effectively killed FSHR+ OVCAR3 tumor cells in a dose-dependent manner compared to mock T cells (FIG. 16B). To test the treatment potential of targeting FSHR in clinical tumors, we treated NSG mice growing identically established FSHR ovarian patient-derived xenograft (PDX) tumors with T cells transduced with FSH-CER or mock vectors. Remarkably, FSH-CER T cells induced complete rejection of orthotopic human ovarian PDX tumors expressing the highest levels of FSHR (FIG. 16A, C, using autologous T cells), as well as those flank PDX expressing moderate FSHR levels (FIG. 16A, D). In both cases, administration of mock T cells into mice growing the same PDX tumors allowed for steady tumor growth. These data support the treatment of human ovarian cancers using FSHR as a target.

(3) FSH-CER T Cells Impact Progression of FSHR Orthotopic Ovarian Tumors in Syngeneic Mice.

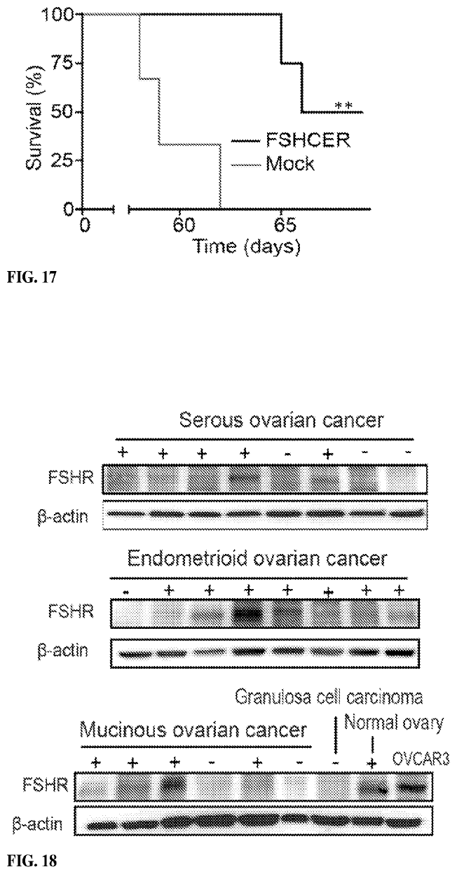

To gain insight into the potential of administering FSH-CER T cells directly into the ovarian TME in syngeneic hosts, we transduced mouse T cells with the murine version of FSH-CER. When two cohorts of mice bearing established orthotopic FSHR ovarian tumors were treated with 2 injections of FSH-CER T cells, all mice receiving mock-transduced T cells succumbed to the disease, while mice having FSH-CER T cells had extended survival (FIG. 17). Notably, in this highly ascitogenic model, FSH-CER T cells did not completely eradicate the tumors. This could be explained by the poor activity of virus-transduced mouse T cells or the immune suppressive effect of the TME. We argue that the moderate anti-tumor effect found in this aggressive model will enable to test if targeting different pathways of ER stress empowers FSH-CER T cells to completely eliminate established ovarian tumors.

(4) Most Human Ovarian Cancers Express FSHR.

Seminal publications support that 50-70% of ovarian cancers express the FSHR. To confirm the relevance of FSHR as a target, we tested the expression of FSHR in 76 human ovarian carcinomas from our established bank using Western-blot. Variable levels of FSHR were noticed in most serous, endometrioid, and mucinous ovarian human tumors (FIG. 18). Also, high levels of FSHR were detected in normal ovary, which was not a concern as ovarian cancer patients receiving FSH-CER T cells will undergo bilateral salpingo-oophorectomy. Results confirm the potential of FSH-CER T cells for ovarian cancer therapy.

(5) Inhibition of IRE1α-XBP1 Promotes T Cell Activity in Tumors.

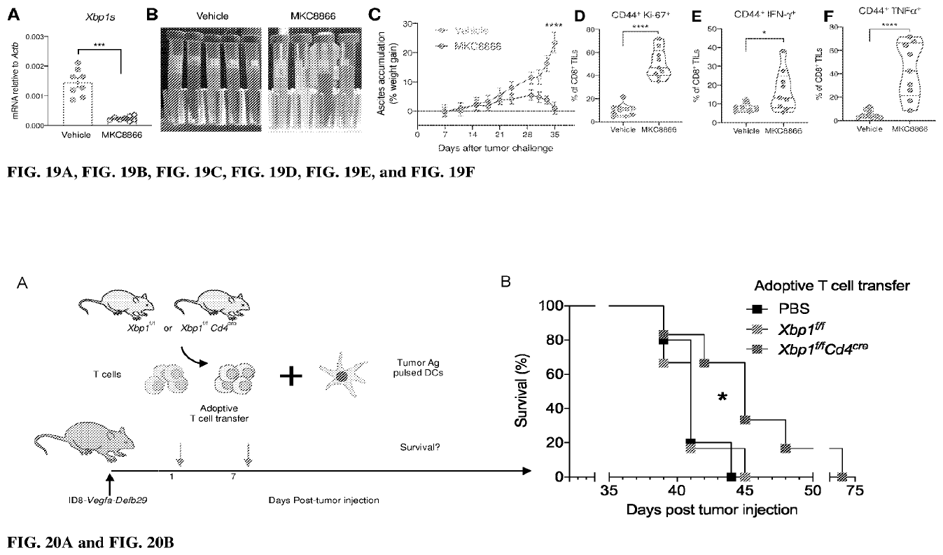

We optimized a therapeutic scheme wherein female mice bearing orthotropic ID8-Defb29/Vegfa tumors for 7 days were treated daily for two weeks with vehicle or MKC8866, a novel small-molecule inhibitor that selectively blocks the RNase domain of IRE1α, impeding the generation of XBP1s. MKC8866 administration rapidly and significantly decreased the levels of IRE1α-generated Xbp1s mRNA in leukocytes from the peritoneal cavity of ID8-Defb29/Vegfa-bearing mice (FIG. 19A). Also, we found impaired generation of hemorrhagic ascites (FIG. 19B) and delayed malignant progression (FIG. 19C) in ovarian cancer hosts receiving MKC8866, compared to vehicle-treated controls.

Loss of IRE1α-XBP1 in intra-tumoral T cells enhanced their metabolic fitness and anti-tumor activity in ovarian tumors. Thus, we assessed whether IRE1α inhibition using MKC8866 modulated the activity of tumor-infiltrating T cell. Consistently, using conditional XBP1-deficient mice, we observed that MKC8866 treatment increased the proportion of proliferating (Ki67+) antigen-experienced (CD44+) CD8+ T cells infiltrating ID8-Defb29/Vegf-A ovarian tumors, while concurrently augmenting their capacity to produce both IFNγ and TNFα in situ (FIG. 19D-F), indicating that treatment with MKC8866 elicits immunostimulatory effects characterized by improved effector capacity in intra-tumoral CD8+ T cells. Notably, intra-tumoral CD8+ T cells in MKC8866-treated mice demonstrated upregulation of PD-1, raising the possibility that therapeutic IRE1a inhibition and checkpoint blockade code induce synergistic anti-tumor effects in ovarian cancer hosts.

(6) IRE1-α-XBP1 Limits Ovarian Cancer-Reactive T Cells:

Ovarian cancer-bearing mice lacking IRE1α-XBP1 selectively in T cells had superior anti-tumor immunity, delayed malignant progression, and increased survival. To test the effect of deleting IRE1α-XBP1 in transferred anti-tumor T cells, we used our established model. Briefly, floxed or Xbp1-deficient T cells were enriched for cancer reactivity through ex vivo incubation with DCs pulsed with ovarian tumor lysates, followed by transfer into mice bearing established ID8-Defb29-Vegfa tumors (FIG. 20A). Our preliminary data show that transfer with Xbp1-deficient polyclonal T cells primed and expanded with these lysates extended host survival in comparison with their floxed (WT) counterparts (FIG. 20B), thus indicating superior therapeutic efficacy of Xbp1-null T lymphocytes.

(7) Deletion of Perk in T Cells or Inhibition of Perk Increases Anti-Tumor Effector Activity.

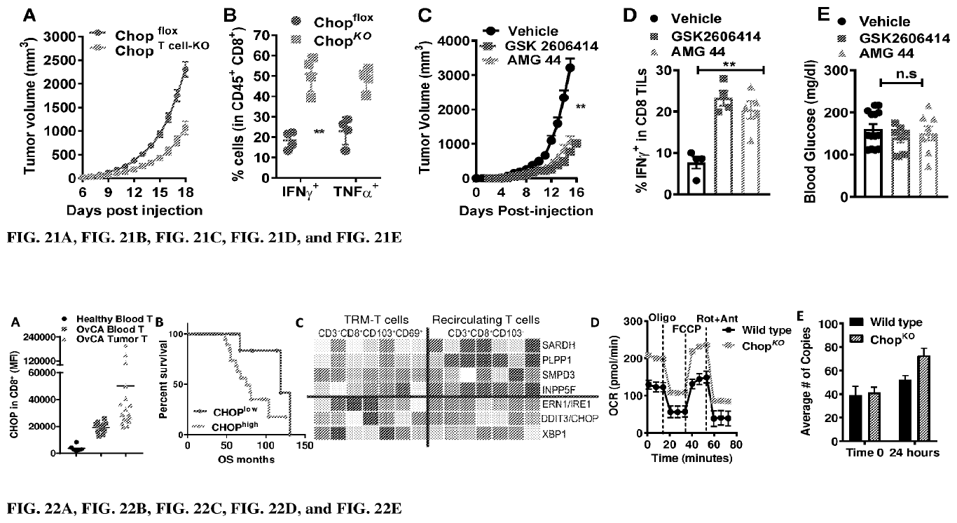

A recent report demonstrated the crucial role of the activation of PERK in the upregulation of Chop in tumor-infiltrating T cells and in activated T cells exposed to ovarian cancer ascites from ID8-Defb29/Vegfa tumors. Moreover, conditional deletion of Chop (FIG. 21A) or PERK in T lymphocytes (Chop^{flox} or Perk^{flox} mice crossed with CD4-Cre recombinase mice) resulted in decreased tumor growth, compared to controls, which correlated with a higher production of IFNγ and TNFα in tumor-infiltrating CD8⁺ T cells (FIG. 21B). Next, we evaluated the anti-tumor therapeutic effects of the small molecule PERK inhibitors, AMG-44 (a highly specific PERK inhibitor) or GSK-2606414 (a dual PERK and RIPK1 kinase inhibitor). Treatment of tumor-bearing mice with PERK inhibitors delayed tumor growth (FIG. 21C) and amplified the frequency of IFNγ-producing CD8⁺ T cells in tumors (FIG. 21D). Previous studies indicated that high doses of GSK-2606414 (150 mg/kg, orally twice a day) induced hyperglycemia by a progressive damage of the pancreatic 13 cells. Results using significant lower doses failed to show alterations in glucose levels in the serum from tumor-bearing mice treated with PERK inhibitors (FIG. 21E). Thus, our results show the intrinsic effect of the PERK-Chop axis in the dysfunction of T cells within tumors; and the therapeutic potential of the inhibition of PERK in tumors as a strategy to boost T cell activity.

(8) Chop Expression in Recirculating TILs Correlates with Poor Clinical Responses.

We aimed at determining whether CD8+ T cells upregulate Chop upon infiltration into the TME. Using a cohort of patients with advanced ovarian cancer and controls, we found higher CHOP levels in CD8+ TILs, compared to autologous peripheral blood CD8+ T cells or lymphocytes from healthy controls (FIG. 22A). Notably, higher levels of nuclear CHOP in CD8+ TILs significantly associated with decreased overall survival in patients with ovarian cancer (median survival 75 vs. 119 months) (FIG. 22B). Since T_{RM} T cells promote higher anti-tumor responses compared with recirculating counterparts, we tested the levels of the key ER stress mediators, CHOP, IRE1α, and XBP1 in a RNAseq established from T_{RM} T cells (CD3+CD8+CD69+CD103+) and recirculating T cells (CD3+CD8+CD69− CD103−) from 7 different ovarian human tumors. In accordance with the argued anti-tumor functionality of T_{RM} T cells, lower levels of the ER stress mediators CHOP (DDIT3), IRE1α (ERN1), and XBP1 were found in T_{RM} T cells compared to recirculating T cells (FIG. 22C). Also, T_{RM} T cells showed a higher expression of transcripts linked to mitochondrial respiration-biogenesis (FIG. 22C), a phenotype also noted in primed Chop^{KO} CD8+ T cells and demonstrated through mitochondrial metabolism analysis using Seahorse (OCR, FIG. 22D) and quantitation of mitochondrial DNA content (FIG. 22E). Thus, our results suggest: 1) Higher expression of CHOP in CD8+ TILs correlates with poor clinical responses; 2) T_{RM} T cells show lower levels of ER stress-related gene markers, compared with recirculating T cells; and 3) Chop^{KO} T cells display higher mitochondrial respiratory activity.

(9) Inhibition of PERK or IRE1α Promotes T_{RM} Expansion and Boosts the Therapeutic Efficacy of FSH-CER T Cells

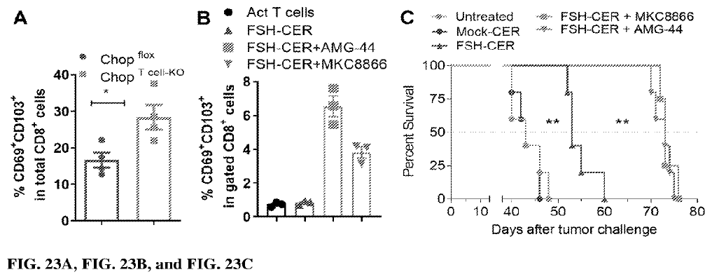

The role of the PERK-Chop in the expansion of tumor T_{RM} cells using conditional T cell Chop-null mice was evaluated. In agreement with the inhibitory effect of Chop in T_{RM} development, higher frequency of T_{RM} cells was detected in tumors from T cell-Chop^{KO} mice compared to controls (FIG. 23A). Next, we tested the effect of the inhibition of PERK (AMG-44) and IRE 1α (MKC8866) during the expansion of IL-2-supplemented human FSH-CER T cells. Higher frequency of FSH-CER T_{RM} cells was found after treatment for 5 days with PERK or IRE1α inhibitors (FIG. 23B), indicating the regulatory effect of these ER stress drivers in the expansion of T_{RM} cells. Also, we evaluated the therapeutic effect of the combination of ER stress-targeting therapy and FSH-CER. Thus, mice bearing FSHR+-ID8-Defb29/Vegfa ovarian tumors for 7 days received daily treatments with MKC8866 (i.p., 300 mg/kg) or AMG-44 (i.p. 12 mg/kg) for one-week, and i.p. transfer of FSH-CER T cells at days 7 and 14 post-tumor challenge. Prolonged survival was found in ovarian cancer hosts receiving FSH-CER T cells and treated for only a week with PERK or IRE1α inhibitors (FIG. 23C), showing the potential of this therapeutic strategy as an approach to enhance the effects of CAR-T cells. We postulate that treatments for longer time periods with ER stress inhibitors will further potentiate the effect of FSH-CER T cells

(10) Effector Activity in Human Ovarian Cancer Depends on Oligo-Clonal T_{RM} TILs.

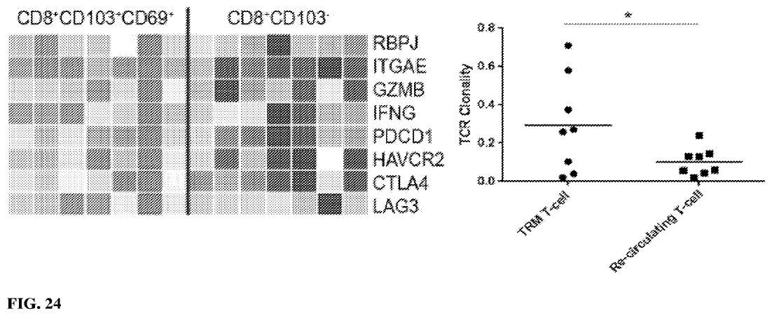

Expansion of tumor T_{RM} T cells is associated with better prognosis in human ovarian cancer. To understand the role of tumor T_{RM} T cells in this malignancy, we FACS-sorted T_{RM} and recirculating T cells from 7 different human advanced serous ovarian carcinomas from our bank and performed RNA-seq (as in FIG. 22C). Supporting previous reports, T_{RM} T cells (˜60% of total CD8+ TILs) showed higher levels of cytolytic GZMB and IFNγ effector mediators, but also overexpressed exhaustion/activation markers PD-1, LAGS, TIM3 or CTLA4 (FIG. 24, left). Unexpectedly, ImmunoSeq TCR repertoire analysis of 8 different specimens showed a very limited overlap of T cell specificities in T_{RM} vs. recirculating CD8+ T cells sorted from the same tumors (ranging from 6% to 20%; average, 11%). Despite differences between patients, the clonality index of T_{RM} CD8+ T cells was significantly higher than that of their CD103− counterparts, suggesting enrichment for tumor-specific populations in T_{RM} cells (FIG. 24, right). These findings support that anti-ovarian cancer immunity is exerted by a distinct population of T_{RM} T cells that, despite signs of exhaustion, express augmented effector levels and exhibit clonality.

f) Determine how Endogenous ER Stress Responses Thwart the Effectiveness of CER-T Cells.

Adoptive immunotherapies using either high-affinity TCR or CAR-T cells have shown remarkable clinical results for the treatment of hematological malignancies. However, it remains uncertain whether these strategies can be effective against solid carcinomas, including ovarian carcinoma, due to the immunoinhibitory TME inherent of these tumors. Our preliminary studies and previous reports show the role of the maladaptive activation of IRE1α-XBP1 and PERK-Chop in tumor-infiltrating T cells and myeloid cells in the suppression of protective T cell immunity. However, the mechanistic interaction between these stress signals, as well as the effect of the intrinsic ablation of IRE1α-XBP1 and/or PERK-Chop in the adoptively transferred T cells vs. the inhibition of these pathways in the TME of the T cell recipients remain to be studied. We hypothesize that the intrinsic priming of IRE1α-XBP1 and/or PERK-Chop in FSH-CER T cells upon homing to ovarian tumors, or the over-activation of IRE1α-XBP1 and/or PERK-Chop in the immunosuppressive TME, limit the protective potential of CAR/CER-T cells. Thus, the major goal of this Aim is to demonstrate that controlling ER stress responses in transferred T cells or the TME can augment the efficacy of adoptively-transferred CER-T cells.

(1) Adoptive Immunotherapy Using UPR-Modified CER-T Cells.

Exploiting our conditional-deficient mice and using clinical protocols routinely employed at Moffitt to generate human CAR-T cells, we will develop a new class of syngeneic mouse FSH-CER-T cells lacking IRE1α-XBP1 and/or PERK-Chop, and will evaluate their function, persistence, and therapeutic efficacy upon transfer into hosts bearing metastatic ovarian cancer. Thus, T cells isolated from our Ern1^{f/f}CD4^{cre} (IRE1α^{KO}), Xbp1^{f/f}Cd4^{cre} (XBP1^{KO}), Eif2ak3^{f/f}CD4^{cre} (PERK^{KO}) or Ddit3^{f/f}Cd4^{cre} (Chop^{KO}) mice, and their corresponding littermate controls, will be transduced with our murine 4-1BB-based FSH-CER construct (FIG. 15). Engineered FSH-CER T cells will be then expanded and sorted on the basis of GFP+ expression at day 7. Then, WT or UPR-modified FSH-CER T cells (CD45.2+) will be transferred (1-2×10⁶/mouse) via i.p. injection into CD45.1+ hosts bearing FSHR+-ID8-Defb29/Vegfa ovarian cancer at days 7 and 14 after tumor challenge. Therapeutic efficacy will be assessed by monitoring tumor growth, malignant progression, ascites accumulation and overall host survival. CER-T cell infiltration, persistence, and cytotoxic capacity will be tested 3, 6, 9, 15, and 21 days after infusion. Extensive functional analyses available in our group, will be used to evaluate the status of the anti-tumor immune responses elicited by the transferred CER-T cells devoid of ER stress sensors. Importantly, using the congenic markers described above will enable us to assess how UPR-modified CER-T cells impact the phenotypic and functional changes of endogenous anti-tumor immune cell populations, memory T cell formation, and host protection. Furthermore, we will evaluate whether ablation of either IRE1α-XBP1 or PERK-Chop induces hyperactivation of the reciprocal arm. Also, we will test whether the simultaneous ablation of IRE1α and PERK (IRE1α^{KO}-PERK^{KO}) or XBP1 and Chop (XBP1^{KO}-Chop^{KO}) further improves the therapeutic activity of CER-T cells, compared with CER-T cells lacking only one axis. Importantly, experiments using CER-T cells devoid of both IRE1α and PERK will further allow us to interrogate the potential compensatory function of the ATF6 branch of the UPR. Together, this sub-aim will define for the first time whether genetic abrogation of IRE1α-XBP1 and/or PERK-Chop signaling in CER-T cells potentiates their protective capacity against metastatic ovarian cancer.

(2) Pharmacological Targeting of IRE1α and PERK to Reprogram the TME During CER-T Cell Therapy.

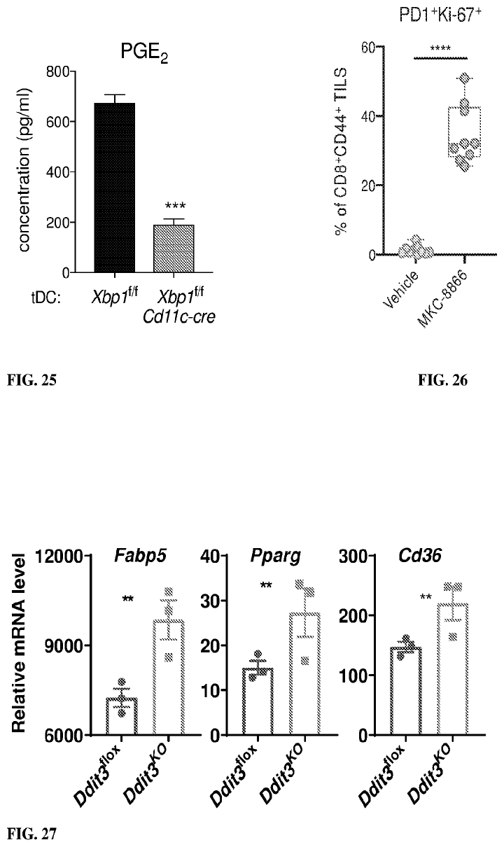

Activation of the UPR in cancer cells mediates adaptation processes in the TME, whereas UPR priming in tumor-myeloid cells promotes their immunoregulatory activity. Indeed, our previous research showed that IRE1α-XBP1 and PERK-Chop promoted myeloid cell suppressive activity in tumors. Our recent findings further reveal a major role for the DC-intrinsic IRE1α-XBP1 in the production of immuno-regulatory PGE2 in the ovarian TME (FIG. 25). Since our preliminary data show an additional intrinsic detrimental role of the UPR in T cell activity, we hypothesize that treatment of ovarian cancer-bearing mice with small-molecule inhibitors of IRE 1α and/or PERK prior to and during infusion with CER-T cells, will induce tumor cell death, overcome immunosuppression in the TME, and significantly augment the activity of FSH-CER T cells. Thus, we will use two safe and selective small-molecule inhibitors of IRE1α: MKC8866 (targets RNAse domain, FIG. 19) and KIRA8 (blocks kinase domain), as well as 2 small-molecule Perk inhibitors: AMG-44 (targets its kinase activity) or GSK2606414 (dual Perk and RIPK1 kinase inhibitor) (FIG. 21E-F). Hosts bearing FSHR+-ID8-Defb29/Vegfa ovarian tumors will undergo daily treatments with non-toxic doses of vehicle vs. MKC8866 (300 mg/kg) vs. KIRA8 (50 mg/kg) vs. AMG-44 (12 mg/kg) vs. GSK2606414 (25 mg/kg) or the different combination of IRE1α and PERK inhibitors, in the presence or the absence of adoptive transfer with FSH-CER T cells. Small-molecule inhibitor treatments will start on day 3 (prophylactic) or day 7 (therapeutic) post-tumor challenge and continue daily throughout the experiment, whereas 1-2×10⁶/mouse FSH-CER T cells will be i.p. transferred at days 7 and 14 after tumor challenge. As main readouts of therapeutic efficacy, we will monitor tumor burden, overall survival, and tumor CER-T cell infiltration, persistence, and cytotoxic capacity (Exp. C.2.1.1). Cellular markers of proliferation, exhaustion, and memory differentiation will be also tested in transferred CER-T cells via FACS. Additionally, we will sort DCs and MDSC from tumor sites and lymphoid organs to quantify the expression of ER stress gene markers, their ability to impair T cell proliferation and IFNγ production, and the levels of the UPR-related immunoinhibitory factors IL-6, PGE₂, Cox-2, mPGES-1, and Arginase I. All required methods are established by our groups. Experiments will define whether CER-T cells show enhanced protective activity in ovarian cancer hosts treated with inhibitors controlling key immunoregulatory signals driven by the ER stress sensors IRE1α and/or PERK.

(3) Assessment of Immune Checkpoints Upon ER Stress Inhibition in Ovarian Cancer Hosts.

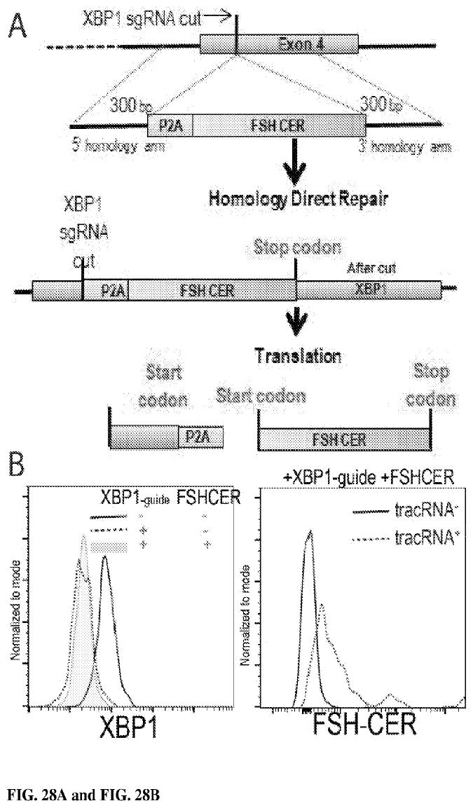

We will determine whether targeting ER stress response pathways during CER-T cell therapy, either genetically or pharmacologically, induces the expression levels of checkpoint mediators in the host as a compensatory mechanism that might promote immune escape during treatment. To this end, we will test the expression of multiple suppressive drivers, such as PD-L1, PD-L2, CD-200 and Galectins in the TME and lymphoid tissues at different time points during treatment. We will simultaneously assess the expression of receptors for inhibitory ligands and common immune checkpoints (PD1, CTLA-4, LAG3, Tim-3, and TIGIT) on tumor-infiltrating T cells. Indeed, we found a substantial elevation of the immune checkpoint PD-1 in mice treated with MKC8866 or conditionally lacking Perk-Chop (FIGS. 26 and 28), likely as an indicator of their recent activation in the TME. Based on these studies, we will evaluate whether treatment with checkpoint blockers maximizes the therapeutic effects of ER stress-targeted CER-T cells, or if this new type of cellular therapy is able to bypass regular mechanisms of T cell exhaustion that normally operate in the TME.

(4) Results and Interpretation.

Deletion of IRE1α-XBP1 or PERK-Chop can intrinsically improve the therapeutic potential and cytotoxic anti-tumor activity of FSH-CER T cells. It is conceivable that CER-T cells simultaneously lacking both IRE1α and PERK or XBP1 and Chop will show superior anti-tumor function, but it is also possible that ablating both pathways at the same time may impact the survival of CER-T cells in the TME. 2) Pharmacological inhibition of IRE1α and PERK in CER-T cell-treated mice with ovarian cancer induce tumor cell death, reprogram the TME into an immunogenic hot tissue, and boost the effectiveness of FSH-CER T cells. Thus, we expect maximal host survival and tumor regression in mice treated with ER stress inhibitors and infused with CER-T cells. 3) Therapeutic combinations synergize with different checkpoint inhibitors.

g) Elucidate the Mechanisms Whereby the IRE1α-XBP1 and PERK-Chop Signaling Pathways Distinctively Control the Metabolism and T_{RM} Differentiation of Adoptively-Transferred CER-T Cells.

We recently uncovered that maladaptive IRE1α-XBP1 activation in T cells limits their glutamine uptake capacity, mitochondrial respiration, and overall anti-ovarian cancer activity. This is of high relevance as optimal mitochondrial activity has emerged as a primary mediator of T cell anti-tumor capacity. Also, our preliminary data show that the PERK-Chop arm of the UPR restricts the expansion of protective T_{RM} T cells in tumors, while also limiting the bio-energetic mitochondrial capacity of T cells (FIG. 22-23). Since T_{RM} T cells display higher ability to induce anti-tumor responses, it is critical to define whether the modulation of ER stress responses could be used to therapeutically expand T_{RM} T cells. We hypothesize that the deletion of IRE1α-XBP1 or PERK-Chop intrinsically augments the anti-cancer effectiveness of FHS-CER-T cells in the adverse TME via two distinct pathways: 1) Loss of IRE1α-XBP1 improves glutamine influx and mitochondrial respiration in ER-stressed T cells in the TME; and 2) PERK ablation inhibits phospho-eIF2α-driven expression of Chop, which transcriptionally represses master mediators that jointly promote T_{RM} T cell development and mitochondrial lipid metabolism. Thus, our results will dissect the mechanisms by which ER stress impacts the efficacy of transferred T cells and pave the way for improving the activity of cellular therapies in solid tumors.

(1) Monitoring Tumor-Induced ER Stress Responses in Infused CER-T Cells:

We will determine the kinetics of ER stress induction in adoptively-transferred CER-T cells to evaluate how progressive disruption of ER homeostasis and maladaptive UPR activation in vivo correlates with metabolic and effector dysfunction in these engineered T cells. CER-T cells will be generated from ERAI reporter mice⁴ available in our group, which express the Venus fluorescent protein only upon ER stress-driven activation of IRE1α. ERAI-based CER-T cells will be manufactured, expanded and adoptively transferred into mice bearing ovarian cancer as described herein. Using congenic markers, we will sort Venus^{negative/low} (IRE1α-inactive) vs. Venus^{mid-high} (IRE1α-active) CER-T cells (both CD4⁺ and CD8⁺) from blood, lymphoid tissues, solid tumors and ascites 1, 3, 5 and 7 days after transfer. Sorted populations demonstrating varying levels of IRE1α activation (Venus levels) will be profiled ex vivo to assess the magnitude of UPR activation and its correlation with mitochondrial respiratory capacity (Seahorse), effector function (IFN-γ production), and expression of memory or exhaustion markers.

(2) IRE1α-XBP1 Signaling Regulates Mitochondrial Function in Transferred CER-T Cells.

Active XBP1 controls the abundance of glutamine carriers and therefore, glutamine influx and mitochondrial respiration, in ER-stressed T cells. Accordingly, we observed that enforcing expression of the glutamine transporter SNAT1 or supplementing glutamine-derived TCA cycle metabolites, such as α-KG, restored mitochondrial respiration and effector function in T cells under glucose deprivation or exposed to patient-derived ovarian cancer ascites, which are conditions that provoke IRE1α-XBP1-driven T cell mitochondrial dysfunction. Thus, we hypothesize that the XBP1-mediated regulation of glutamine transporter abundance and the consequent restriction in glutamine consumption inhibits the mitochondrial activity and functionality of CER-T cells in the tumor after adoptive transfer. Using the genetically-modified CER-T cells, we will evaluate the levels of the major glutamine transporters ASCT2, SNAT1 and SNAT2, the mitochondrial respiratory capacity (Seahorse), the uptake of ¹³C-labeled glutamine, and the effector/tumoricidal capacity of WT vs. IRE1α-XBP1 CER-T cells after transfer into mice bearing FSHR+ ovarian tumors, following methods optimized by our group. To specifically confirm that reduced glutamine transporter levels limit the optimal function of transferred T cells in ovarian tumors, we will overexpress SNAT1 in transferred FSH-CER-T cells and we will evaluate their therapeutic potential by assessing their ability to recognize and kill FSHR+ malignant cells (IFNγ and granzyme B ELISPOT and cytotoxicity assays), as well as their expression of T cell memory and exhaustion markers. Together, these studies will establish whether maladaptive activation of T cell-intrinsic XBP1 suppresses the mitochondrial function of adoptively transferred FSH-CER-T cells in ovarian cancer hosts.

(3) PERK-Chop Limits Differentiation of FSH-CER T Cells into T_{RM} Subset.

Our unpublished studies showed lower ER stress responses in tumor-T_{RM} T cells compared with recirculating T cells; and a potential role of PERK-Chop in the expansion of T_{RM} T cells in ovarian tumors and in the regulation of mitochondrial T cell activity (FIG. 22-23). To extend our data in transferred CER-T cells, we will evaluate the activation status of IRE1α-XBP1 and PERK-Chop, and the mitochondrial respiratory capacity (Seahorse) in T_{RM}-CER T cells (CD3+CD8+CD69+CD103+) vs. recirculating counterparts (CD3+CD8+CD69-CD103−) before and after transfer into mice bearing FSHR+ ovarian tumors. Based on our preliminary data, we are likely to find lower ER stress activation and higher mitochondrial activity in T_{RM} CER-T cells compared with their recirculating counterparts. Next, we will evaluate whether the ablation of PERK-Chop in FSH-CER T cells promotes their expansion into T_{RM} CD8+ T cells in tumors. Mice bearing established FSHR+-ID8-Defb29/Vegfa tumors will be transferred with FSH-CER T cells from Chop^{KO} or Perk^{KO} mice and 9-21 days later tested for the percentage of tumor-T_{RM} vs. recirculating FSH-CER T cells. We expect to find higher frequency of T_{RM} T cells in those CER-T cells lacking PERK-Chop. In this event, we will determine whether the elevated anti-tumor activity induced by FSH-CER T cells devoid of PERK-Chop is caused through an expansion of T_{RM} T cells. Control, Chop^{KO}, or Perk^{KO} T cells will be transduced with FSH-CER and after expansion, T_{RM} T cells will be depleted using anti-CD103-mAbs, and the negative fraction transferred into mice bearing established FSHR+-ovarian tumors. Also, we will treat CER-T cell-transferred mice with FTY720, which will prevent the egress and trafficking of T cells from lymphoid tissues to tumor sites and will therefore enable a specific assessment of the protective capacity of tumor-resident populations differentiated in response to loss of PERK-Chop. Reduced therapeutic effects in mice treated with PERK-Chop^{KO} CER-T cells treated with CD103-depleting antibodies or FTY720 will confirm the protective role for T_{RM} T cells. These studies will enable the development of novel strategies to promote the development of T_{RM}-enriched T cell-based therapies effective against metastatic ovarian tumors.

(4) PERK-Chop Regulates T_{RM} Cell Expansion.

The results shown herein indicated a regulatory role for Chop in the development of tumor-T_{RM} cells (FIG. 23). This is important as the primary pathways driving the expansion of T_{RM} T cells in tumors remain largely unexplored. A seminal report showed that skin CD8+T_{RM} use mitochondrial 13 oxidation of exogenous fatty acids to support their longevity and anti-viral function. T_{RM} T cell development was driven by a master group of mediators that controlled lipid oxidation, including fatty-acid-binding proteins 4 and 5 (FABP4 and FABP5), peroxisome proliferator-activated receptor gamma (PPARγ), and lipid transporter CD3623. We postulate that the activation of PERK-Chop in FSH-CER T cells undergoing ER stress restricts their differentiation into protective T_{RM} T cells through transcriptional inhibition of these primary mediators of lipid metabolism and T_{RM} T cell development. In fact, our RNAseq preliminary studies showed increased expression of Fabp5, Pparg, and CD36 in activated Chop^{KO} CD8+ T cells, compared to controls (FIG. 27). Also, prediction analysis developed using the TFTG-BD and ChIP-Base v2.0 databases indicated consensus Chop binding sites on the promoter regions of Pparg and Fabp5. We will test the ability of Chop to repress the transcription of Pparg and Fabp5 in 293T cells co-transfected with vectors coding for CMV-driven Chop cDNA (Addgene), Pparg or Fabp5-promoter fused to firefly luciferase, and control TK-driven renilla luciferase. Data showing reduced firefly/renilla activity after expression of Chop will suggest its negative effect on Pparg or Fabp5 transcription. If this prediction is true, we will next test the endogenous binding of Chop to Pparg or Fabp5 promoters in activated CER-T cells treated with malignant ascites or the ER stress inducer thapsigargin, as well in tumor-FSH-CER-T cells transferred into mice bearing FSHR+ ovarian tumors, by ChIP assays. Data showing a higher binding of Chop to Pparg or Fabp5 promoters will show the endogenous regulation of these genes by the PERK-Chop pathway.

To further understand the negative role of PERK-Chop in the expression of key drivers of T_{RM} T cells, we will measure the expression of Fabp5, Pparg, and CD36 in flox, Perk^{KO}, or Chop^{KO} tumor-CER-T cells, expecting a significant increase in FSH-CER T cells deficient of Perk or Chop, compared to flox controls. Next, we will determine the importance of the repression of Pparg or Fabp5 in the elevated development of T_{RM} T cells found after ablation of PERK-Chop. Thus, Perk^{KO} or Chop^{KO} FSH-CER-T cells will be specifically silenced for Pparg, Fabp5, and/or CD36 using mock vs. CRISPR-Cas9 vectors. Next, the transduced FSH-CER T cells will be adoptively transferred into mice bearing established FSHR+-ID8-Defb29/Vegfa tumors, and tested for the expansion of T_{RM} vs. recirculating transferred T cells in tumors, mitochondrial respiratory activity, and therapeutic efficacy against FSHR+-tumors. Results showing a lower expansion of T_{RM} CER-T cells, impaired respiratory capacity, and reduced anti-tumor reactivity in PERK-Chop-deficient FSH-CER T cells after silencing of Pparg, Fabp5, and/or CD36, will indicate a mechanistic crosstalk between this UPR axis and key drivers of T_{RM} metabolic development. Together, the proposed research will identify a new strategy to promote the differentiation and activity of adoptively transferred CER-T cells into a T_{RM} subtype.

h) Test the Postulate that the Modulation of IRE1α-XBP1 and/or PERK-Chop Signaling During the In Vitro Generation of CER-T Cells Significantly Improves their Therapeutic Potential In Vivo.

During ER stress, multiple signals converge to activate NF-κB in epithelial cells. We argue that it is crucial to confirm the occurrence of these pathways in T cells, because the effector differentiation, survival and metabolic fitness could be influenced by ER stress. For instance, NF-κB has been proposed as a major signal factor driving the effector differentiation in memory T cells. This is important because CAR-T cells from responding patients are enriched in memory-related genes, whereas T cells from non-responders upregulate programs involved in effector differentiation. Importantly, CD28-based CARs induce dramatically different phenotypes in terms of metabolic activity and effector differentiation, compared to 4-1BB-CARs. The effects of ER stress during the generation of CAR-T cell infusion products might therefore depend on the co-stimulatory domain utilized. The goal of these studies is to elucidate how modulation of ER stress pathways could be optimized to generate an optimal infusion product with superior anti-tumor activity after in vivo transfer. While we realize that some degree of NF-κB activity and even ER stress responses could be needed for the development of effective T cells, we postulate that persistent IRE1α-XBP1 and/or PERK-Chop activation during the engineering of FSH-CER T cells alters their metabolic fitness and memory differentiation, and therefore their subsequent effectiveness in vivo. We argue that these negative effects will occur to a much higher degree in CD28-based CARs. Accordingly, CRISPR-mediated ablation or pharmacological modulation of these pathways will forge a superior infusion of chimeric receptor-expressing T cells in solid tumors.

(1) In Vitro Inhibition of IRE1α-XBP1 and/or PERK-Chop Regulates the Activity of FSH-CER T Cells.

We will engineer T cells from our IRE1^{-/-}-CD4^{Cre} and PERK^{-/-}-CD4^{Cre} mice, as well as from IRE1^{-/-}PERK^{-/-}-CD4^{Cre} double transgenic and control littermates, to express our fully murine (4-1BB-based) FSH-CER constructs. In parallel, we will retrovirally transduce human T cells from ≥5 healthy donors to express our human FSH-CER constructs, in the presence of different concentrations of the IRE1 inhibitor MKC8866 vs. the PERK inhibitor AMG-44 vs. the chemical chaperones TUDCA and 4-PBA that promote the resolution of ER stress, and combinations of MKC8866 and/or TUDCA/4-PBA and/or AMG-44 vs. vehicle. Absolute CER-T cell CD4/CD8 yields, apoptosis-death (Annexin V/PI), effector activity, and acquisition of attributes of memory and T_{RM} T cells by mouse and human T cells will be assessed at days 5, 7 and 1444. Also, for all conditions, the magnitude of NF-κB activation upon modulation of ER stress will be tested through Western-blot for phospho-NF-κB p65 (activated form). Next, we will develop murine and human FSH-CER T cells under conditions of genetic and/or pharmacological inhibition of ER stress that result in superior anti-tumor activity, as well as in the absence of treatments. NSG mice challenged with FSHR+ OVCAR3 flank tumors and immunocompetent B6 mice orthotopically injected with FSHR-transduced ID8-Defb28/Vegfa cells (≥10 mice/group) will be then treated at days 7 and 14 with 10⁶-10⁷ FSH-CER T cells. Tumor growth and survival will be monitored, as readout of differential in vivo effectiveness. These data will be compared to in vitro cytotoxic potential against FSHR+ target cells immediately before administration. Also, we will quantify the accumulation of FSH-CER T cells at tumor beds, as well as the production of IFN-γ and GZMB through intracellular staining. Persistence will be finally monitored in different groups of treated tumor-bearing mice every 7 days, until terminal disease in our syngeneic system and up 30 days in NSG mice. Together, these studies define the effects of modulating ER stress on the generation of optimal CAR/CER T cell infusion products that elicit superior anti-tumor activity.

(2) Distinct Effects of Genetic and Pharmacological Inhibition of IRE1α Vs. PERK in the Metabolic Fitness of FSH-CER T Cells.

Metabolism dictates the fate of anti-tumor T cells independently of antigen specificity, and our collaborative work has conclusively demonstrated that IRE-1α-XBP1 and PERK-CHOP blunt metabolic fitness of ovarian cancer-reactive T cells. We will identify how pharmacological modulation of different ER stress signals influences the metabolic profile of CER-T cells in the infusion product. We will generate mouse CER-T cells from WT and UPR-modified mice. In parallel, we will engineer human T cells from ≥5 healthy donors with (4-1BB-based) human FSH-CER constructs, in the presence of different concentrations of MKC8866, AMG-44, TUDCA, MKC8866 plus TUDCA, AMG-44 plus TUDCA, or vehicle. As readouts of glucose metabolism, we will test GLUT1 levels in all groups of FSH-CER T cells at days 7, 10 and 14. Using Seahorse bioenergetics analyzers, we will also monitor OCR, extracellular acidification rate (ECAR) and OCR/ECAR ratio, as readouts of mitochondrial and glycolytic activity, in the presence vs. the absence of glucose. T cell mitochondrial morphology and mass will be additionally monitored, as we published. To assess the effects of modulating ER stress in T cell fatty acid metabolism, crucial in the TME, we will quantify metabolites of FA catabolism (i.e., acetylcarnitine, palmitoylcarnitine, and ketone body 3-hydroxybutyrate) by liquid chromatography-mass spectrometry, using assays optimized at Moffitt Proteomics Core. We will also treat FSH-CER T cells created under different conditions with fenofibrate, a PPAR-α agonist that increases FA catabolism, or etomoxir (ETO), an irreversible inhibitor of Cpt1 that decreases mitochondrial FAO. FA uptake and OCR/ECAR will be quantified in the presence of human/mouse ovarian cancer ascites. Because the choice of the co-stimulatory domain critically influences the metabolic profile of CAR T cells, we will also generate FSH-CER constructs in which 4-1BB will be replaced by the intracellular domain of CD28. The effects of genetic/pharmacological modulation of ER stress on the metabolic fitness of these cells will be compared to our originally prioritized 4-1BB-based CERs. Studies will define how distinct signals of ER stress influence the metabolic fitness of CER T cells, which will equip them to resist the abrasive milieu driven by the ovarian TME.

(3) Non-Viral Integration of FSH-CER Constructs with Concurrent Ablation of XBP1.

Emerging studies indicate that CAR/TCR/CER constructs can be effectively expressed in human T cells while avoiding viral vectors by using CRISPR and homology-directed repair (HDR). To circumvent the use of viral vectors and express our FSH-CER constructs while targeting ER stress, we have designed a new system to integrate a 1,386 bp DNA cassette immediately after the start codon of XBP1. This cassette encodes all the subunits of the human FSH-CER sequence, downstream of a self-excising 2A peptide. The cellular machinery that drives the constitutive expression of XBP1 in T cells will ensure the expression of the FSH-CER, while the stop codon at position 1,383 and relatively long sequence of the construct will prevent the transcription/translation of both spliced and non-spliced forms of XBP1 (FIG. 28A). Importantly, XBP1-deficient T cells do not show defects in proliferation, while exhibiting superior anti-tumor activity. In a pilot study, we used a fluorescently-labeled (550 nm) tracRNA, plus our published crRNA oligo with the XBP1-specific guide, followed by the tracrRNA fusion domain. Co-electroporation of CAS9-based ctRNP complexes with an HDR template encoding P2A+ FSH-CER+ two 300 bp homology arms, designed to integrate FSH-CER in frame with XBP1 gene at exon 4, was performed using our Neon protocol. As proof-of-concept, this approach reduced XBP1s in T cells, while turning-on the CG□ subunit of FSH on the T cell surface (FIG. 28B). Thus, Xbp1− FSH-CER+/Xbp1+ FSH-CER-T cells will be expanded in response to: 1) our FSHR+ artificial APCs (aAPCs); 2) aAPCs coated with anti-CD3/CD28 mAbs; or 3) anti-CD3/CD28 beads. In parallel, anti-CD3/CD28-primed T cells will be transduced mimicking clinical protocols. For all groups, acquisition of memory phenotypes (i.e., CD27, CCR7, CD122); stem-like attributes (i.e., TCF1, CXCR5); superior metabolic profile (i.e., OxPhos, glycolysis, fatty acids); higher CD8/CD4 ratios; elevated expansion/survival; and increased in vivo effectiveness will be compared as a function of: 1) XBP1 ablation; and 2) T cell priming through 4-1BB vs. CD28 (FSH-CER).

Example 3: Chop and sXBP1 Flow Cytometry Analysis of Patient CAR T Cells

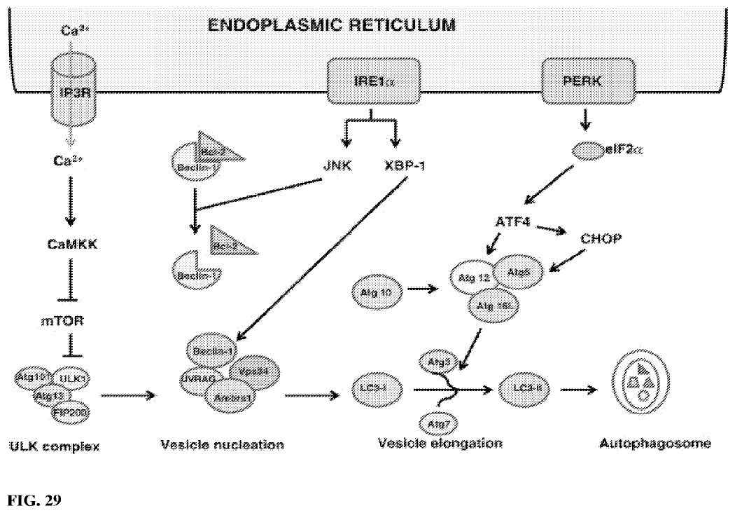

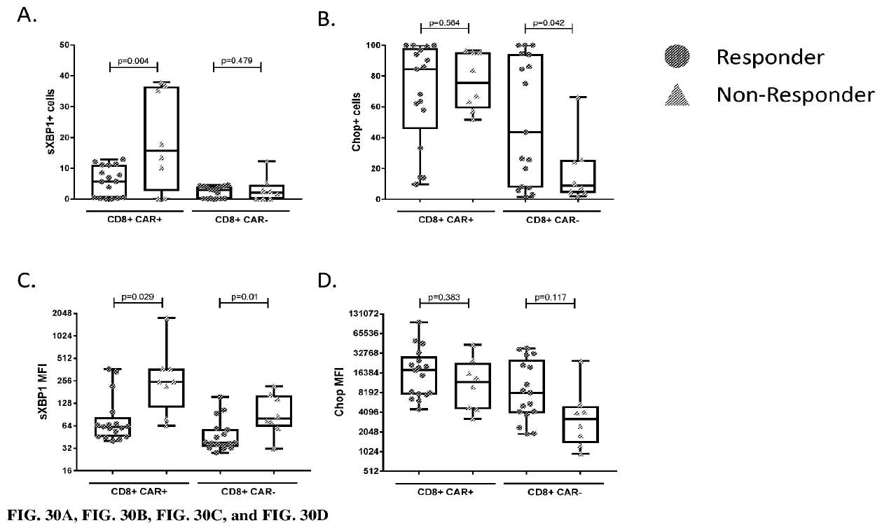

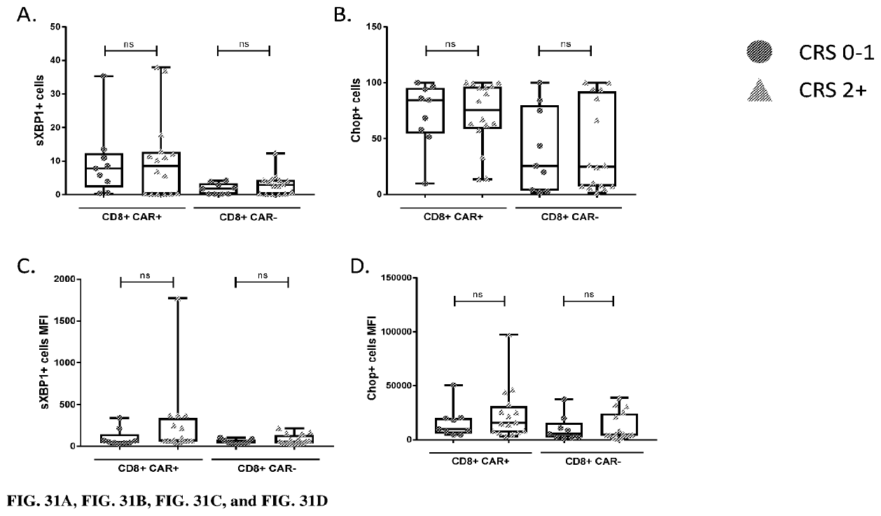

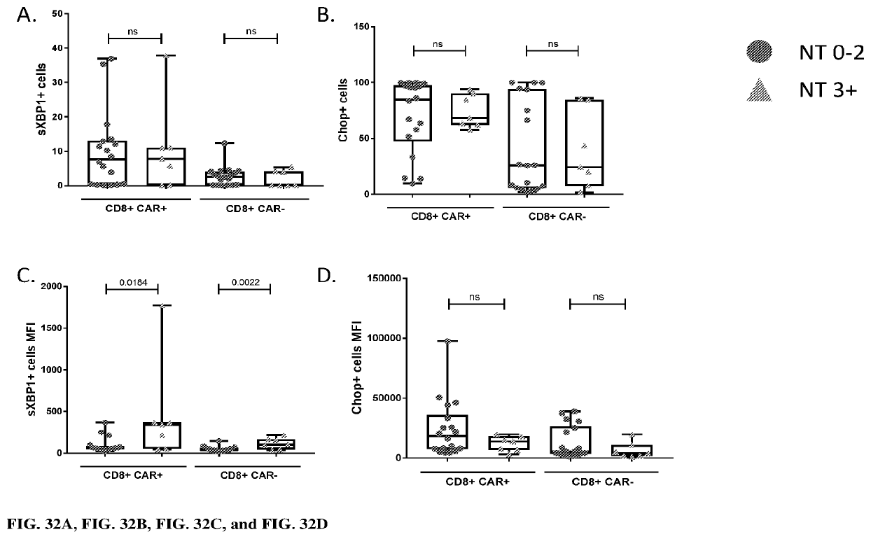

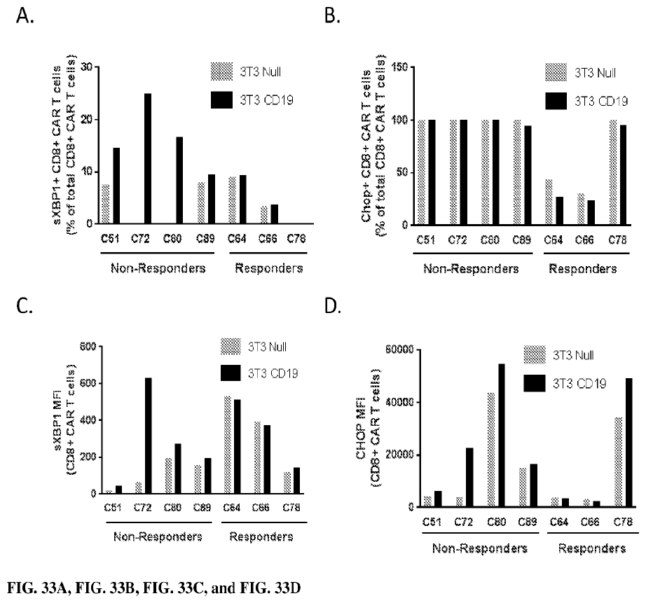

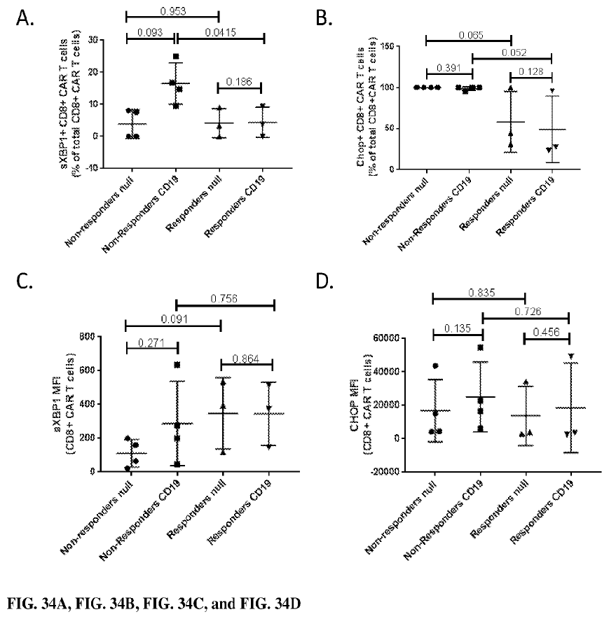

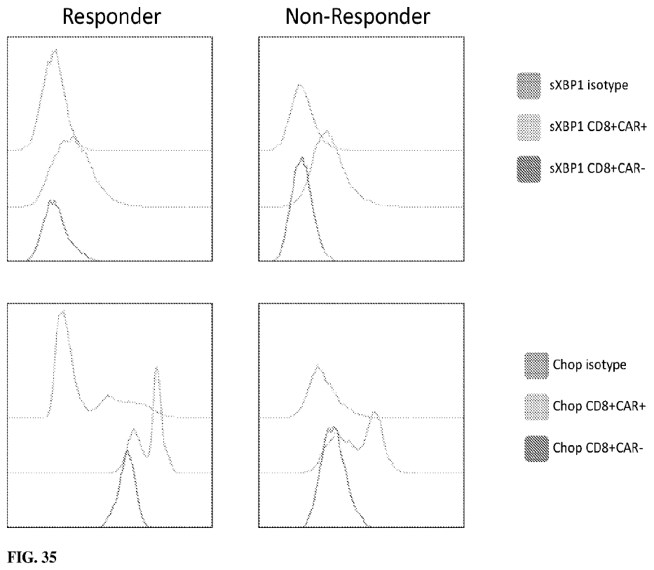

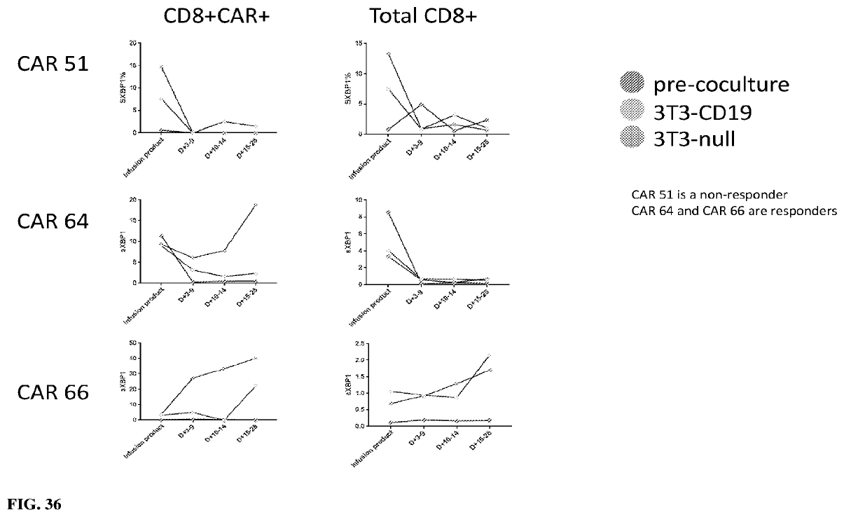

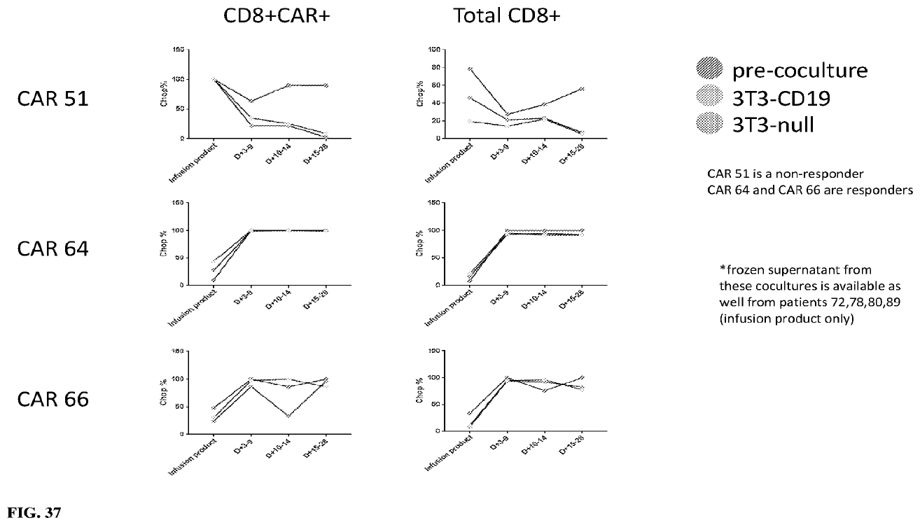

IRE1α and PERK have multiple downstream modulators that ultimately effect vesicle nucleation (IRE1α) and vesicle elongation. As shown in FIG. 29, XBP1 is immediately downstream of IRE1α and Chop is down stream of PERK. FIGS. 30-32 show the evaluation of sXBP1 and Chop by flow cytometry in CAR+ and CAR-T cells. Non-responder, red triangle defined as those not achieving or not remaining in remission at day +90; responder, blue circle defined as those achieving and not progressing prior to day +90. Next, sXBP1 and Chop were evaluated by flow cytometry in CD8+ CAR T cells collected from the product bag from seven axicabtagene ciloleucel treated DLBCL patients. Cells were stimulated with tumor cell line targets, either those transduced to express CD19 target (3T3 CD19) or control without (3T3 null), for 24 hours then analyzed (FIGS. 33 and 34). Non-responder is defined as those not achieving or not remaining in remission at day +90; responder is defined as those achieving and not progressing prior to day +90.

Example 4: Perk Ablation on Melanoma Cells

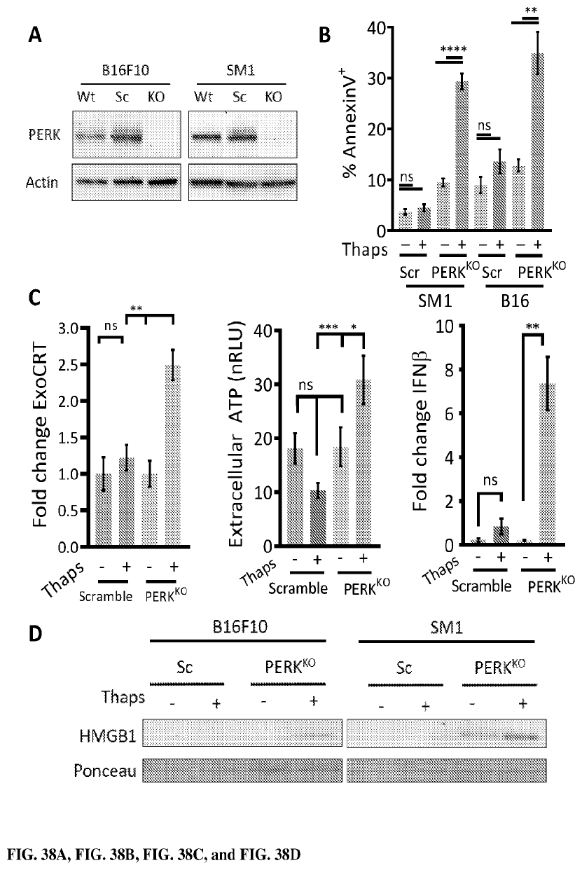

To see the effect of Perk ablation on Melanoma cells, B16-F10 and SM1 melanoma cells were transduced with Crispr-Cas 9 coding vectors targeting the expression of PERK or a Scramble (sc) control. After selection of specific clones, PERK expression was detected by immunoblot. Representative finding of N=3. FIGS. 38B, 38C, and 38D show cells from (FIG. 38A) were treated with the ER stress inducer Thapsigargin and monitored for the levels of Apoptosis marker Annexin V (FIG. 38B), as well as the immunogenic cell death mediators, cellular membrane translocated Calreticulin (ExoCRT), extracellular ATP, expression of IFNb mRNA (38C), and accumulation of extracellular HMGB1 (38D). Experiments were independently repeated at least 4 times.

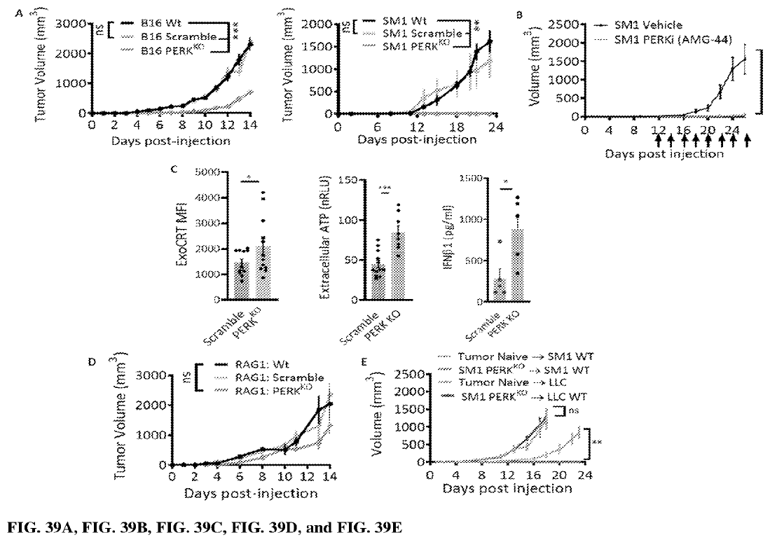

Deletion of PERK in melanoma cells impaired tumor growth and elicited immunogenic cancer cell death and sustained protective T cell immunity (FIG. 39). B16-F10 or SM1 melanoma cells were implanted s.c. into C57BL/6 mice (at least N=10/group) and tumor volume evaluated daily using calipers. C57BL/6 mice bearing SM1 tumors for 12 days received treatments with PERK inhibitor AMG-44 (12 mg/kg, i.p., every other day), followed by assessment of tumor progression (FIG. 39B). Tumor cell suspensions from mice bearing Scramble or B16-F10 tumors for 15 days were evaluated for the expression of the immunogenic cell death mediators, cellular membrane translocated Calreticulin (ExoCRT), extracellular ATP, and IFNb mRNA (FIG. 39C). Additionally, SM1 melanoma cells were implanted into immunodeficient Rag2^{KO} mice and monitored for tumor growth every day using calipers (FIG. 39D). Additionally, naïve C57BL/6 mice and C57BL/6 mice that had previously rejected PERK^{KO}-SM1 tumors were challenged in the opposite flank with LLC or WT-SM1 tumor cells (FIG. 39E). Next, mice were followed for tumor volume. Results show that mice that had previously rejected PERK^{KO}-SM1 tumors were entirely resistant to the WT-SM1 tumor challenge, but not to LLC injection, indicating the induction specific and persistent immune memory against the primary tumor. Experiments were independently repeated at least 3 times.

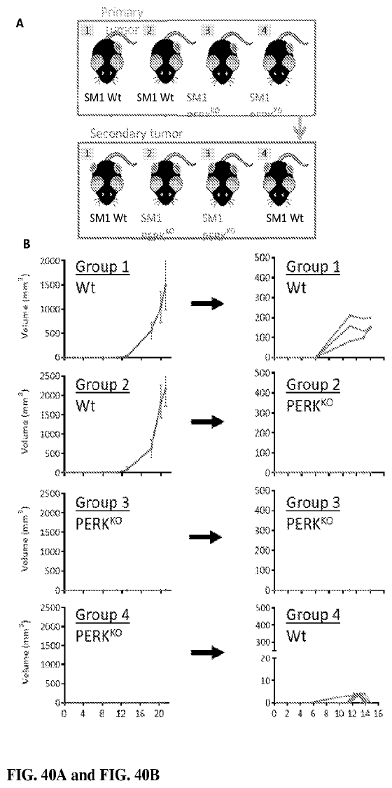

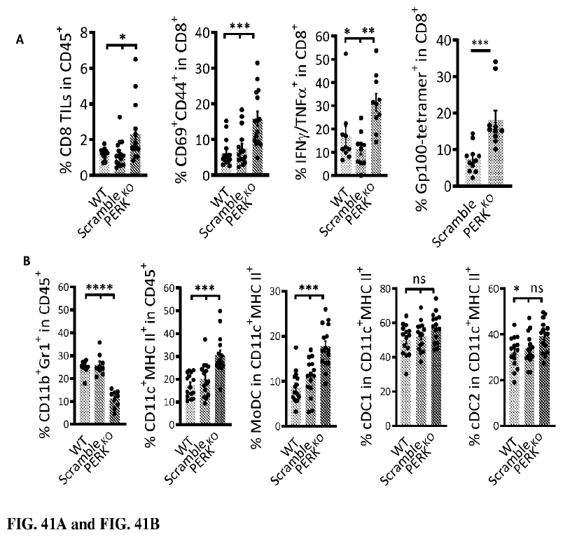

FIGS. 40A and 40B show that elimination of PERK in melanoma cells induces abscopal anti-tumor effects that protect regrow of tumors. FIG. 40A shows C57BL/6 mice were primarily injected s.c. with Wild type (WT, Group 1 and 2) or PERK-deficient (PERK^{KO}, Group 3 and 4) SM1 tumors. Ten days later, mice received a secondary injection in the opposite flank of WT (Group 1 and 4) or PERK^{KO} (Group 2 and 3) SM1 tumors. FIG. 40B shows growth of the primary and secondary tumors was monitored using calipers. Results showed the development of abscopal anti-tumor events in mice that received primary PERK^{KO} tumors against WT counterparts located in the opposite flank. Conversely, no abscopal effect was observed in mice bearing primary WT-SM1 tumors.

We next investigated the effect that ablation of PERK in melanoma cells has on T cell immunity. We observed that ablation of PERK induces protective T cell immunity and reprograms the immunosuppressive myelopoiesis into development of Monocytic-derived DCs. C57BL/6 mice were injected s.c. with Wild type (WT), Scramble, or PERK-deficient (PERK^{KO}) B16-F10 tumors. Fifteen days later, tumors were collected and monitored via FACs for the frequency of CD8⁺ T cells (in CD45⁺ cells); recently activated CD44⁺ CD69⁺ T cells (in CD8⁺ cells); polyfunctional IFNg⁺ TNFa⁺ T cells (in CD8⁺ cells); and melanoma gp100 tetramer⁺ specific T cells (in CD8⁺ cells) (FIG. 41A). Results indicate the increased expansion of T cells, recently activated T cells, effector T cells and tumor-specific T cells in PERK^{KO} B16-F10 tumors, as compared to Scramble and WT counterparts. Tumor suspensions were additionally assessed for the presence of MDSC (CD11b⁺ Gr1⁺ in CD45⁺ cells); Dendritic cells (CD11c⁺ MHC-II⁺ in CD45⁺ cells), Monocytic derived Dendritic cells (MoDCs, CD45⁺ CD11b⁺→CD11c⁺ MHC-II⁺→CD103⁺ Ly6C⁺); DC1s (CD45⁺ CD11b⁻→CD11c⁺ MHC-II⁺→CD103⁺ Ly6C⁻); and DC2s (CD45⁺ CD11b⁺→CD11c⁺ MHCII⁺→CD103⁻ Ly6C⁺). We found a lower accumulation of MDSCs in PERK^{KO} tumors, which correlated with a higher accumulation of MoDCs (FIG. 41B).

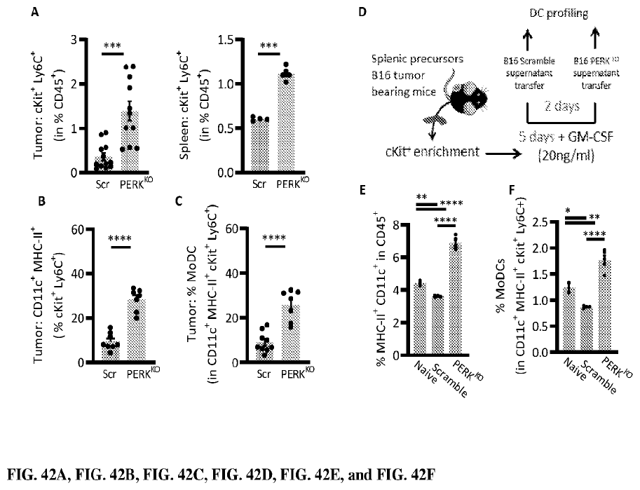

We also observed that PERK elimination in melanoma cells induces the expansion of cKit⁺ Ly6c⁺ myeloid precursors that differentiate into Monocytic-derived DCs (MoDCs). C57BL/6 mice were injected s.c. with Scramble (Scr) or PERK-deficient (PERK^{KO}) B16-F10 tumors. Fifteen days later, the frequency of cKit⁺ Ly6c⁺ Myeloid Precursors (in CD45⁺) was monitored via FACs in tumors (left) and spleen (right) (FIG. 42A). In addition, we evaluated the expansion in tumors of DCs (CD11c⁺ MHC-II⁺ in cKit⁺ Ly6c⁺ cells) (FIG. 42B) and MoDCs (CD103⁺ in CD11c⁺ cKit⁺ Ly6c⁺ cells) (FIG. 42C). Results show the accumulation of MoDCs among the cKit⁺ Ly6c⁺ Precursors. To determine whether the cKit⁺ Ly6c⁺ cells serve as the precursors for the detected MoDCs in PERK^{KO} B16-F10 tumors, cKit⁺ cells were isolated from spleens of mice bearing Scr or PERK^{KO} B16-F10 tumors, cultured for 2 days in the presence of GM-CSF, and monitored for the expansion of DCs (FIG. 42E) and MoDCs (FIG. 42F). Results indicate the heightened expansion of MoDCs from splenic myeloid precursors isolated from PERK^{KO} B16-F10 tumors, compared to those harvested from Scr controls, suggesting the expansion of a highly immunostimulatory subset of myeloid precursors after elimination of PERK in tumors. Merged results are from 3 distinct repeats.

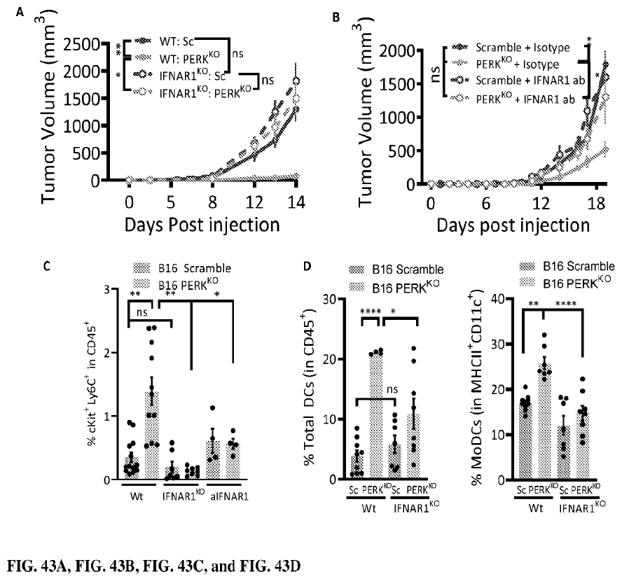

To determine how PERK ablation is mediated in melanoma cells, and whether this mediation is through Type I IFN, C57BL/6 and type I interferon receptor knockout) (IFNAR1^{KO} mice were injected s.c. with Scramble (Sc) or PERK-deficient (PERK^{KO}) SM1 tumors and followed for tumor progression (N=10). The results show that type I IFN signaling mediates the anti-tumor effects induced by the ablation of PERK in melanoma cells (FIG. 43A). FIG. 43B shows C57BL/6 mice bearing Sc or PERK^{KO} B16-F10 tumors were treated with anti-IFNAR1 blocking antibodies and followed for tumor growth. Results show that elimination of IFNAR1 in the hosts or blockade of IFNAR1 overcomes the anti-tumor effects induced by the deletion of PERK in tumor cells, suggesting a key role of host-derived IFNAR1 in the anti-tumor effects induced by PERK deletion in tumors. FIG. 43C shows elimination of IFNAR1 in the hosts or ab-based IFNAR1 blockade prevents the tumor accumulation of myeloid cKit⁺ Ly6c⁺ precursors induced by the ablation of PERK in B16-F10 tumor cells. FIG. 43D shows expansion of DCs and MoDCs in WT and IFNAR1^{KO} mice carrying Sc or PERK^{KO} B16-F10 tumors. Similar to the delayed expansion of myeloid precursors (FIG. 43C), deletion of IFNAR1 in the hosts overcomes the accumulation of DC and MoDCs induced by the elimination of PERK in the tumor cell compartment.

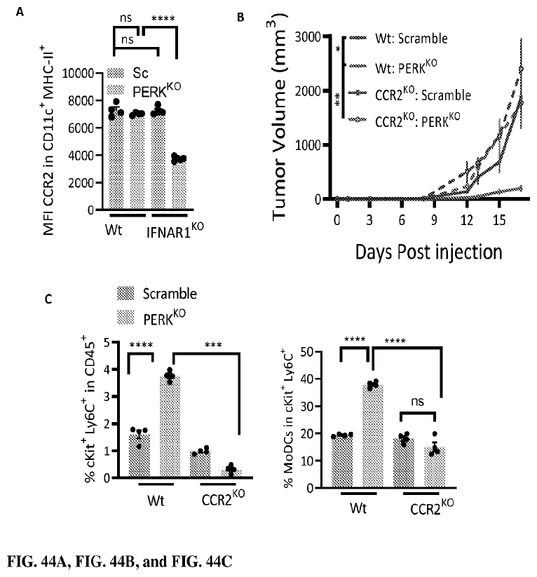

To determine if control of the infiltration of immune stimulatory myeloid precursors in PERK^{KO} tumors is by type I IFNs-dependent CCR2, CCR2 expression in tumor-related DCs (CD116⁺ MHC-II⁺) from Scramble (Sc) or PERK-deficient (PERK^{KO}) B16-F10 tumors were injected into C57BL/6 or type I interferon receptor knockout (IFNAR1^{KO}) mice. Results show that deletion of IFNAR1 prevents the expression of CCR2 in DCs expanding in PERK^{KO} tumors (FIG. 44A). Additionally, C57BL/6 and CCR2^{KO} mice were injected with Sc or PERK^{KO} B16-F10 tumors and followed for tumor progression. Results show that elimination of CCR2 in mice blunts the anti-tumor effects observed in PERK^{KO} tumors (FIG. 44B). It was also found that elimination of CCR2 in mice overcomes the expansion of Myeloid Precursors (cKit⁺ Ly6c⁺) and MoDCs induced by the elimination of PERK in B16-F10 tumor cells (FIG. 44C). *p<0.05, ****p<0.001. ns: p≥0.05

### F. References

Ahn, J., Son, S., Oliveira, S. C., and Barber, G. N. (2017). STING-Dependent Signaling Underlies IL-10 Controlled Inflammatory Colitis. Cell Rep 21, 3873-3884.
Ahn, J., Xia, T., Konno, H., Konno, K., Ruiz, P., and Barber, G. N. (2014). Inflammation-driven carcinogenesis is mediated through STING. Nat Commun 5, 5166.
Atkins, C., Liu, Q., Minthorn, E., Zhang, S. Y., Figueroa, D. J., Moss, K., Stanley, T. B., Sanders, B., Goetz, A., Gaul, N., et al. (2013). Characterization of a novel PERK kinase inhibitor with antitumor and antiangiogenic activity. Cancer Res. 73, 1993-2002.
Bettigole, S. E., and Glimcher, L. H. (2015). Endoplasmic reticulum stress in immunity. Annu Rev Immunol 33, 107-138.
Bettigole, S. E., Lis, R., Adoro, S., Lee, A. H., Spencer, L. A., Weller, P. F., and Glimcher, L. H. (2015). The transcription factor XBP1 is selectively required for eosinophil differentiation. Nat Immunol 16, 829-837.
Bhattacharya, S., HuangFu, W. C., Dong, G., Qian, J., Baker, D. P., Karar, J., Koumenis, C., Diehl, J. A., and Fuchs, S. Y. (2013). Anti-tumorigenic effects of Type 1 interferon are subdued by integrated stress responses. Oncogene 32, 4214-4221.
Bi, M., Naczki, C., Koritzinsky, M., Fels, D., Blais, J., Hu, N., Harding, H., Novoa, I., Varia, M., Raleigh, J., et al. (2005). ER stress-regulated translation increases tolerance to extreme hypoxia and promotes tumor growth. EMBO J. 24, 3470-3481.
Bobrovnikova-Marjon, E., Grigoriadou, C., Pytel, D., Zhang, F., Ye, J., Koumenis, C., Cavener, D., and Diehl, J. A. (2010). PERK promotes cancer cell proliferation and tumor growth by limiting oxidative DNA damage. Oncogene 29, 3881-3895.
Cao, Y., Trillo-Tinoco, J., Sierra, R. A., Anadon, C., Dai, W., Mohamed, E., Cen, L., Costich, T. L., Magliocco, A., Marchion, D., et al. (2019). ER stress-induced mediator C/EBP homologous protein thwarts effector T cell activity in tumors through T-bet repression. Nat Commun 10, 1280.
Carroll, E. C., Jin, L., Mori, A., Munoz-Wolf, N., Oleszycka, E., Moran, H. B. T., Mansouri, S., McEntee, C. P., Lambe, E., Agger, E. M., et al. (2016). The Vaccine Adjuvant Chitosan Promotes Cellular Immunity via DNA Sensor cGAS-STING-Dependent Induction of Type I Interferons. Immunity 44, 597-608.
Chang, T. K., Lawrence, D. A., Lu, M., Tan, J., Harnoss, J. M., Marsters, S. A., Liu, P., Sandoval, W., Martin, S. E., and Ashkenazi, A. (2018). Coordination between Two Branches of the Unfolded Protein Response Determines Apoptotic Cell Fate. Mol Cell 71, 629-636 e625.
Chen, X., Iliopoulos, D., Zhang, Q., Tang, Q., Greenblatt, M. B., Hatziapostolou, M., Lim, E., Tam, W. L., Ni, M., Chen, Y., et al. (2014). XBP1 promotes triple-negative breast cancer by controlling the HIFlalpha pathway. Nature 508, 103-107.
Chevet, E., Hetz, C., and Samali, A. (2015). Endoplasmic reticulum stress-activated cell reprogramming in oncogenesis. Cancer Discov 5, 586-597.
Condamine, T., Dominguez, G. A., Youn, J. I., Kossenkov, A. V., Mony, S., Alicea-Torres, K., Tcyganov, E., Hashimoto, A., Nefedova, Y., Lin, C., et al. (2016). Lectin-type oxidized LDL receptor-1 distinguishes population of human polymorphonuclear myeloid-derived suppressor cells in cancer patients. Sci Immunol 1.
Condamine, T., Kumar, V., Ramachandran, I. R., Youn, J. I., Celis, E., Finnberg, N., El-Deiry, W. S., Winograd, R., Vonderheide, R. H., English, N. R., et al. (2014). ER stress regulates myeloid-derived suppressor cell fate through TRAIL-R-mediated apoptosis. J. Clin. Invest 124, 2626-2639.
Corrales, L., Glickman, L. H., McWhirter, S. M., Kanne, D. B., Sivick, K. E., Katibah, G. E., Woo, S. R., Lemmens, E., Banda, T., Leong, J. J., et al. (2015). Direct Activation of STING in the Tumor Microenvironment Leads to Potent and Systemic Tumor Regression and Immunity. Cell Rep 11, 1018-1030.
Corzo, C. A., Cotter, M. J., Cheng, P., Cheng, F., Kusmartsev, S., Sotomayor, E., Padhya, T., McCaffrey, T. V., McCaffrey, J. C., and Gabrilovich, D. I. (2009). Mechanism regulating reactive oxygen species in tumor-induced myeloid-derived suppressor cells. J. Immunol. 182, 5693-5701. Cubillos-Ruiz, J. R., Silberman, P. C., Rutkowski, M. R., Chopra, S., Perales-Puchalt, A., Song, M., Zhang, S., Bettigole, S. E., Gupta, D., Holcomb, K., et al. (2015). ER Stress Sensor XBP1 Controls Anti-tumor Immunity by Disrupting Dendritic Cell Homeostasis. Cell 161, 1527-1538.
Cullinan, S. B., and Diehl, J. A. (2004). PERK-dependent activation of Nrf2 contributes to redox homeostasis and cell survival following endoplasmic reticulum stress. J Biol Chem 279, 20108-20117.
Cullinan, S. B., Zhang, D., Hannink, M., Arvisais, E., Kaufman, R. J., and Diehl, J. A. (2003). Nrf2 is a direct PERK substrate and effector of PERK-dependent cell survival. Mol Cell Biol 23, 7198-7209.
Deng, L., Liang, H., Xu, M., Yang, X., Burnette, B., Arina, A., Li, X. D., Mauceri, H., Beckett, M., Darga, T., et al. (2014). STING-Dependent Cytosolic DNA Sensing Promotes Radiation-Induced Type I Interferon-Dependent Antitumor Immunity in Immunogenic Tumors Immunity 41, 843-852.
Dong, H., Adams, N. M., Xu, Y., Cao, J., Allan, D. S. J., Carlyle, J. R., Chen, X., Sun, J. C., and Glimcher, L. H. (2019). The IRE1 endoplasmic reticulum stress sensor activates natural killer cell immunity in part by regulating c-Myc. Nat Immunol 20, 865-878.
Gabrilovich, D. I., Ostrand-Rosenberg, S., and Bronte, V. (2012). Coordinated regulation of myeloid cells by tumours. Nat Rev Immunol 12, 253-268.
Glodde, N., Bald, T., van den Boorn-Konijnenberg, D., Nakamura, K., O'Donnell, J. S., Szczepanski, S., Brandes, M., Eickhoff, S., Das, I., Shridhar, N., et al. (2017). Reactive Neutrophil Responses Dependent on the Receptor Tyrosine Kinase c-MET Limit Cancer Immunotherapy. Immunity 47, 789-802.e789.
Hambrock, A., Loffler-Walz, C., and Quast, U. (2002). Glibenclamide binding to sulphonylurea receptor subtypes: dependence on adenine nucleotides. Br J Pharmacol 136, 995-1004.
Harding, H. P., Novoa, I., Zhang, Y., Zeng, H., Wek, R., Schapira, M., and Ron, D. (2000). Regulated translation initiation controls stress-induced gene expression in mammalian cells. Mol. Cell 6, 1099-1108.
Hetz, C., and Papa, F. R. (2018). The Unfolded Protein Response and Cell Fate Control. Mol Cell 69, 169-181.
Hurst, K. E., Lawrence, K. A., Essman, M. T., Walton, Z. J., Leddy, L. R., and Thaxton, J. E. (2019). Endoplasmic Reticulum Stress Contributes to Mitochondrial Exhaustion of CD8(+) T Cells. Cancer Immunol Res.
Ishikawa, H., and Barber, G. N. (2008). STING is an endoplasmic reticulum adaptor that facilitates innate immune signalling. Nature 455, 674-678.
Iwakoshi, N. N., Pypaert, M., and Glimcher, L. H. (2007). The transcription factor XBP-1 is essential for the development and survival of dendritic cells. The Journal Of Experimental Medicine 204, 2267-2275.
Janssens, S., Pulendran, B., and Lambrecht, B. N. (2014). Emerging functions of the unfolded protein response in immunity. Nat. Immunol. 15, 910-919.
Kumar, V., Patel, S., Tcyganov, E., and Gabrilovich, D. I. (2016). The Nature of Myeloid-Derived Suppressor Cells in the Tumor Microenvironment. Trends Immunol 37, 208-220.
Lee, B. R., Chang, S. Y., Hong, E. H., Kwon, B. E., Kim, H. M., Kim, Y. J., Lee, J., Cho, H. J., Cheon, J. H., and Ko, H. J. (2014). Elevated endoplasmic reticulum stress reinforced immunosuppression in the tumor microenvironment via myeloid-derived suppressor cells. Oncotarget 5, 12331-12345.
Lemos, H., Mohamed, E., Huang, L., Ou, R., Pacholczyk, G., Arbab, A. S., Munn, D., and Mellor, A. L. (2016). STING Promotes the Growth of Tumors Characterized by Low Antigenicity via IDO Activation. Cancer Res 76, 2076-2081.
Liang, H., Deng, L., Hou, Y., Meng, X., Huang, X., Rao, E., Zheng, W., Mauceri, H., Mack, M., Xu, M., et al. (2017). Host STING-dependent MDSC mobilization drives extrinsic radiation resistance. Nat Commun 8, 1736.
Lu, M., Lawrence, D. A., Marsters, S., Acosta-Alvear, D., Kimmig, P., Mendez, A. S., Paton, A. W., Paton, J. C., Walter, P., and Ashkenazi, A. (2014). Opposing unfolded-protein-response signals converge on death receptor 5 to control apoptosis. Science 345, 98-101.
Lu, X., Homer, J. W., Paul, E., Shang, X., Troncoso, P., Deng, P., Jiang, S., Chang, Q., Spring, D. J., Sharma, P., et al. (2017). Effective combinatorial immunotherapy for castration-resistant prostate cancer. Nature 543, 728-732.
Maas, N. L., and Diehl, J. A. (2015). Molecular pathways: the PERKs and pitfalls of targeting the unfolded protein response in cancer. Clin Cancer Res 21, 675-679.
Martinon, F., Chen, X., Lee, A.-H., and Glimcher, L. H. (2010). TLR activation of the transcription factor XBP1 regulates innate immune responses in macrophages. Nature Immunology 11, 411-418.
Mohamed, E., Al-Khami, A. A., and Rodriguez, P. C. (2018). The cellular metabolic landscape in the tumor milieu regulates the activity of myeloid infiltrates. Cell Mol Immunol 15, 421-427.
Nakagawa, H., Umemura, A., Taniguchi, K., Font-Burgada, J., Dhar, D., Ogata, H., Zhong, Z., Valasek, M. A., Seki, E., Hidalgo, J., et al. (2014). ER stress cooperates with hypernutrition to trigger TNF-dependent spontaneous HCC development. Cancer Cell 26, 331-343.
Olagnier, D., Brandtoft, A. M., Gunderstofte, C., Villadsen, N. L., Krapp, C., Thielke, A. L., Laustsen, A., Peri, S., Hansen, A. L., Bonefeld, L., et al. (2018). Nrf2 negatively regulates STING indicating a link between antiviral sensing and metabolic reprogramming Nat Commun 9, 3506.
Ozcan, U., Yilmaz, E., Ozcan, L., Furuhashi, M., Vaillancourt, E., Smith, R. O., Gorgun, C. Z., and Hotamisligil, G. S. (2006). Chemical chaperones reduce ER stress and restore glucose homeostasis in a mouse model of type 2 diabetes. Science 313, 1137-1140.
Perales-Puchalt, A., Svoronos, N., Rutkowski, M. R., Allegrezza, M. J., Tesone, A. J., Payne, K. K., Wickramasinghe, J., Nguyen, J. M., O'Brien, S. W., Gumireddy, K., et al. (2017). Follicle-Stimulating Hormone Receptor Is Expressed by Most Ovarian Cancer Subtypes and Is a Safe and Effective Immunotherapeutic Target. Clin Cancer Res 23, 441-453.
Pytel, D., Majsterek, I., and Diehl, J. A. (2016). Tumor progression and the different faces of the PERK kinase. Oncogene 35, 1207-1215.
Raber, P. L., Thevenot, P., Sierra, R., Wyczechowska, D., Halle, D., Ramirez, M. E., Ochoa, A. C., Fletcher, M., Velasco, C., Wilk, A., et al. (2014). Subpopulations of myeloid-derived suppressor cells impair T cell responses through independent nitric oxide-related pathways. Int. J. Cancer 134, 2853-2864.
Reimold, A. M., Iwakoshi, N. N., Manis, J., Vallabhajosyula, P., Szomolanyi-Tsuda, E., Gravallese, E. M., Friend, D., Grusby, M. J., Alt, F., and Glimcher, L. H. (2001). Plasma cell differentiation requires the transcription factor XBP-1. Nature 412, 300-307.
Rodriguez, P. C., Ernstoff, M. S., Hernandez, C., Atkins, M., Zabaleta, J., Sierra, R., and Ochoa, A. C. (2009). Arginase I-producing myeloid-derived suppressor cells in renal cell carcinoma are a subpopulation of activated granulocytes. Cancer Res. 69, 1553-1560.
Rojas-Rivera, D., Delvaeye, T., Roelandt, R., Nerinckx, W., Augustyns, K., Vandenabeele, P., and Bertrand, M. J. M. (2017). When PERK inhibitors turn out to be new potent RIPK1 inhibitors: critical issues on the specificity and use of GSK2606414 and GSK2656157. Cell Death Differ 24, 1100-1110.
Rutkowski, M. R., Stephen, T. L., Svoronos, N., Allegrezza, M. J., Tesone, A. J., Perales-Puchalt, A., Brencicova, E., Escovar-Fadul, X., Nguyen, J. M., Cadungog, M. G., et al. (2015). Microbially driven TLR5-dependent signaling governs distal malignant progression through tumor-promoting inflammation. Cancer Cell 27, 27-40.
Shang, W., Tang, Z., Gao, Y., Qi, H., Su, X., Zhang, Y., and Yang, R. (2017). LncRNA RNCR3 promotes Chop expression by sponging miR-185-5p during MDSC differentiation. Oncotarget 8, 111754-111769.
Sharma, M. D., Rodriguez, P. C., Koehn, B. H., Baban, B., Cui, Y., Guo, G., Shimoda, M., Pacholczyk, R., Shi, H., Lee, E. J., et al. (2018). Activation of p53 in Immature Myeloid Precursor Cells Controls Differentiation into Ly6c(+)CD103(+) Monocytic Antigen-Presenting Cells in Tumors. Immunity 48, 91-106.e106.
Shin, S., Wakabayashi, N., Misra, V., Biswal, S., Lee, G. H., Agoston, E. S., Yamamoto, M., and Kensler, T. W. (2007). NRF2 modulates aryl hydrocarbon receptor signaling: influence on adipogenesis. Mol Cell Biol 27, 7188-7197.
Sierra, R. A., Trillo-Tinoco, J., Mohamed, E., Yu, L., Achyut, B. R., Arbab, A., Bradford, J. W., Osborne, B. A., Miele, L., and Rodriguez, P. C. (2017). Anti-Jagged Immunotherapy Inhibits MDSCs and Overcomes Tumor-Induced Tolerance. Cancer Res 77, 5628-5638.
Singhal, S., Bhojnagarwala, P. S., O'Brien, S., Moon, E. K., Garfall, A. L., Rao, A. S., Quatromoni, J. G., Stephen, T. L., Litzky, L., Deshpande, C., et al. (2016). Origin and Role of a Subset of Tumor-Associated Neutrophils with Antigen-Presenting Cell Features in Early-Stage Human Lung Cancer. Cancer Cell 30, 120-135.
Sliter, D. A., Martinez, J., Hao, L., Chen, X., Sun, N., Fischer, T. D., Burman, J. L., Li, Y., Zhang, Z., Narendra, D. P., et al. (2018). Parkin and PINK1 mitigate STING-induced inflammation. Nature 561, 258-262.
Smith, A. L., Andrews, K. L., Beckmann, H., Bellon, S. F., Beltran, P. J., Booker, S., Chen, H., Chung, Y. A., D'Angelo, N. D., Dao, J., et al. (2015). Discovery of 1H-pyrazol-3(2H)-ones as potent and selective inhibitors of protein kinase R-like endoplasmic reticulum kinase (PERK). J Med Chem 58, 1426-1441.
Song, M., Sandoval, T. A., Chae, C. S., Chopra, S., Tan, C., Rutkowski, M. R., Raundhal, M., Chaurio, R. A., Payne, K. K., Konrad, C., et al. (2018). IRE1alpha-XBP1 controls T cell function in ovarian cancer by regulating mitochondrial activity. Nature 562, 423-428.
Sultan, H., Trillo-Tinoco, J., Rodriguez, P., and Celis, E. (2017). Effective antitumor peptide vaccines can induce severe autoimmune pathology. Oncotarget 8, 70317-70331.
Svoronos, N., Perales-Puchalt, A., Allegrezza, M. J., Rutkowski, M. R., Payne, K. K., Tesone, A. J., Nguyen, J. M., Curiel, T. J., Cadungog, M. G., Singhal, S., et al. (2017). Tumor Cell-Independent Estrogen Signaling Drives Disease Progression through Mobilization of Myeloid-Derived Suppressor Cells. Cancer Discov 7, 72-85.
Tanaka, Y., and Chen, Z. J. (2012). STING specifies IRF3 phosphorylation by TBK1 in the cytosolic DNA signaling pathway. Sci Signal 5, ra20.
Thaxton, J. E., Wallace, C., Riesenberg, B., Zhang, Y., Paulos, C. M., Beeson, C. C., Liu, B., and Li, Z. (2017). Modulation of Endoplasmic Reticulum Stress Controls CD4(+) T-cell Activation and Antitumor Function. Cancer Immunol Res 5, 666-675.
Thevenot, P. T., Sierra, R. A., Raber, P. L., Al-Khami, A. A., Trillo-Tinoco, J., Zarreii, P., Ochoa, A. C., Cui, Y., Del Valle, L., and Rodriguez, P. C. (2014). The stress-response sensor chop regulates the function and accumulation of myeloid-derived suppressor cells in tumors Immunity 41, 389-401.
Trento, C., Marigo, I., Pievani, A., Galleu, A., Dolcetti, L., Wang, C. Y., Serafini, M., Bronte, V., and Dazzi, F. (2017). Bone marrow mesenchymal stromal cells induce nitric oxide synthase-dependent differentiation of CD11b(+) cells that expedite hematopoietic recovery. Haematologica 102, 818-825.
Urra, H., Dufey, E., Avril, T., Chevet, E., and Hetz, C. (2016). Review: Endoplasmic Reticulum Stress and the Hallmarks of Cancer. Trends in Cancer 2, 252-262.
Veglia, F., Perego, M., and Gabrilovich, D. (2018). Myeloid-derived suppressor cells coming of age. Nat Immunol 19, 108-119.
Walter, P., and Ron, D. (2011). The unfolded protein response: from stress pathway to homeostatic regulation. Science 334, 1081-1086.
Wang, C., Guan, Y., Lv, M., Zhang, R., Guo, Z., Wei, X., Du, X., Yang, J., Li, T., Wan, Y., et al. (2018). Manganese Increases the Sensitivity of the cGAS-STING Pathway for Double-Stranded DNA and Is Required for the Host Defense against DNA Viruses. Immunity 48, 675-687 e677.
Weber, J., Gibney, G., Kudchadkar, R., Yu, B., Cheng, P., Martinez, A. J., Kroeger, J., Richards, A., McCormick, L., Moberg, V., et al. (2016). Phase I/II Study of Metastatic Melanoma Patients Treated with Nivolumab Who Had Progressed after Ipilimumab. Cancer Immunol Res 4, 345-353.
West, A. P., Khoury-Hanold, W., Staron, M., Tal, M. C., Pineda, C. M., Lang, S. M., Bestwick, M., Duguay, B. A., Raimundo, N., MacDuff, D. A., et al. (2015). Mitochondrial DNA stress primes the antiviral innate immune response. Nature 520, 553-557.
Woo, S. R., Fuertes, M. B., Corrales, L., Spranger, S., Furdyna, M. J., Leung, M. Y., Duggan, R., Wang, Y., Barber, G. N., Fitzgerald, K. A., et al. (2014). STING-dependent cytosolic DNA sensing mediates innate immune recognition of immunogenic tumors. Immunity 41, 830-842.
Wu, H., Wei, L., Fan, F., Ji, S., Zhang, S., Geng, J., Hong, L., Fan, X., Chen, Q., Tian, J., et al. (2015). Integration of Hippo signalling and the unfolded protein response to restrain liver overgrowth and tumorigenesis. Nat Commun 6, 6239.
Wu, J., Sun, L., Chen, X., Du, F., Shi, H., Chen, C., and Chen, Z. J. (2013). Cyclic GMP-AMP is an endogenous second messenger in innate immune signaling by cytosolic DNA. Science 339, 826-830.
Xu, M. M., Pu, Y., Han, D., Shi, Y., Cao, X., Liang, H., Chen, X., Li, X. D., Deng, L., Chen, Z. J., et al. (2017). Dendritic Cells but Not Macrophages Sense Tumor Mitochondrial DNA for Cross-priming through Signal Regulatory Protein alpha Signaling. Immunity 47, 363-373 e365.
Yan, D., Wang, H. W., Bowman, R. L., and Joyce, J. A. (2016). STATS and STAT6 Signaling Pathways Synergize to Promote Cathepsin Secretion from Macrophages via IRE1alpha Activation. Cell Rep 16, 2914-2927.
Yu, Q., Zhao, B., Gui, J., Katlinski, K. V., Brice, A., Gao, Y., Li, C., Kushner, J. A., Koumenis, C., Diehl, J. A., and Fuchs, S. Y. (2015). Type I interferons mediate pancreatic toxicities of PERK inhibition. Proc Natl Acad Sci USA 112, 15420-15425.
Zhang, C., Shang, G., Gui, X., Zhang, X., Bai, X. C., and Chen, Z. J. (2019). Structural basis of STING binding with and phosphorylation by TBK1. Nature.

### G. Sequences

SEQ ID NO: 1
TGCGTAGTCTGGAGCTATGGGUUUUAGAGCUAGAAA
SEQ ID NO: 2
SIINFEKL

1-37. (canceled)

38. An immunotherapy comprising PKR-like ER kinase (PERK) inhibitor,
endoplasmic reticulum (ER) stress inhibitor, and/or inositol-requiring enzyme 1α (IRE1α) inhibitor; preferably wherein the PERK inhibitor, ER stress inhibitor or IRE1α inhibitor comprises a RNAi; small molecule; peptide; protein; or antibody that targets PERK, IRE1α, thapsigargin (Thap), Eif2ak3, CC12, GR1, or Nfe2l2; or tumor-infiltrating Eif2ak3^{KO-Lyz2} CD11b⁺ Gr1⁺ cells.

39. The immunotherapy of claim 38, further comprising an adoptive immunotherapy.

40. The immunotherapy of claim 39, wherein the adoptive immunotherapy comprises the administration of chimeric antigen receptor (CAR) T cells, CAR NK cells, tumor infiltrating lymphocytes (TILs), and/or marrow infiltrating lymphocytes (MILs).

41. A method of treating, inhibiting, reducing, ameliorating, and/or preventing a cancer and/or metastasis in a subject comprising administering to a subject the immunotherapy of claim 38.

42. The method of claim 39, wherein the cells of the adoptive immunotherapy are obtained from an autologous or allogeneic donor source.

43. The method of claim 39, wherein the cells of the adoptive immunotherapy are contacted with the PERK inhibitor, ER Stress inhibitor, and/or an IRE1α inhibitor ex vivo prior to administration to the subject or in vivo.

44. A method of reprogramming an immunosuppressive myelopoiesis in a tumor in a subject or myeloid-derived suppressor cells (MDSC) in a tumor in a subject into immunostimulatory myeloid cells comprising administering to the subject the immunotherapy of claim 38.

45. A method of stimulating endogenous T cells in a subject to kill a tumor comprising administering to a subject the immunotherapy of claim 38; wherein the administration of the PERK inhibitor, ER stress inhibitor or IRE1α inhibitor reduces or reduces the effects of one or more immunosuppressive elements in the tumor.

46. A method of increasing the efficacy of an adoptive immunotherapy said method comprising administering to the subject the immunotherapy of claim 38; wherein the administration of the PERK inhibitor, ER stress inhibitor or IRE1α inhibitor reprograms immunosuppressive myelopoiesis in a tumor thereby boosting the efficacy of the adoptive immunotherapy.

47. The method of increasing the efficacy of an adoptive immunotherapy of claim 46, further comprising obtaining a donor population of cells for immunotherapy.

48. The method of increasing the efficacy of an adoptive immunotherapy claim 47, wherein the donor cell population comprises chimeric antigen receptor (CAR) T cells, CAR NK cells, tumor infiltrating lymphocytes (TILs), and/or marrow infiltrating lymphocytes (MILs) from an autologous or allogeneic donor source.

49. An engineered immune cell transduced to express Chimeric Endocrine Receptors (CERs) that express one or more subunits of the follicle-stimulating hormone (FSH).

50. The engineered immune cell of claim 49, wherein immune cell comprises a CAR NK cell, CAR NK T cell, or CAR T cell.

51. A method of treating a FSH-receptor positive (FSHR+) tumors in a subject comprising administering to the subject a the engineered immune cell of claim 49.

52. A method of increasing the efficacy of an engineered immune cell of claim 49 comprising contacting the immune cell with a PERK inhibitor, ER stress inhibitor, and/or IRE 1α inhibitor.

53. A method of treating a FSH-receptor positive (FSHR+) tumors in a subject comprising administering to the subject the engineered immune cell of claim 49.

54. A method of assessing responsiveness to adoptive immunotherapy comprising obtaining adoptively transferred immune cells from a recipient subject and measuring the amount of spliced XBP-1 in the adoptively transferred immune cells, wherein a high level of XBP-1 relative to a control indicates the subject is not responsive to the adoptive immunotherapy.

55. A method of treating, inhibiting, reducing, ameliorating, and/or preventing a cancer and or metastasis in a subject comprising administering to a subject an adoptive immunotherapy and monitoring the amount of spliced XBP-1 in the adoptively transferred immune cells, wherein the level of spliced XBP-1 relative to a control is indicative of responsiveness to the adoptive immunotherapy; and wherein a high level of spliced XBP-1 relative to a control indicates that the recipient subject is not responsive to the adoptive immunotherapy.

56. The method of claim 55, wherein the level of spliced XBP-1 relative to a control is high, said method further comprises administering to the subject a PERK inhibitor, ER Stress inhibitor, and/or an IRE1α inhibitor.
